(19) United States
(12) Patent Application Publication
(10) Pub. No.: US 2025/0163453 A1
(43) Pub. Date: May 22, 2025

(54) MAIZE EVENT DP-051291-2 AND METHODS FOR DETECTION THEREOF

(71) Applicant: PIONEER HI-BRED INTERNATIONAL, INC., JOHNSTON, IA (US)

(72) Inventors: HEATHER MARIE CHRISTENSEN, ANKENY, IA (US); BIN CONG, JOHNSTON, IA (US); VIRGINIA CRANE, DES MOINES, IA (US); ALBERT L. LU, WEST DES MOINES, IA (US); TIMOTHY MABRY, WHITE HEATH, IL (US); KRISTEN DENISE RINEHART KREBS, ANKENY, IA (US); GARY A. SANDAHL, WEST DES MOINES, IA (US)

(73) Assignee: PIONEER HI-BRED INTERNATIONAL, INC., JOHNSTON, IA (US)

(21) Appl. No.: 18/513,905

(22) Filed: Nov. 20, 2023

Related U.S. Application Data
(60) Provisional application No. 63/384,613, filed on Nov. 22, 2022.

(51) Int. Cl. C12N 15/82; C12Q 1/6895

(52) U.S. Cl. CPC C12N 15/8286; C12Q 1/6895; C12Q 2600/13

(57) ABSTRACT

Embodiments disclosed herein relate to the field of plant molecular biology, specifically to DNA constructs for conferring insect resistance to a plant. Embodiments disclosed herein relate to insect resistant corn plant containing event DP-051291-2, and to assays for detecting the presence of event DP-051291-2 in samples and compositions thereof.

### CROSS REFERENCE

This application claims the benefit of U.S. Provisional Application No. 63/384,613, filed on Nov. 22, 2022, the disclosure of which is incorporated herein by reference in its entirety.

### REFERENCE TO SEQUENCE LISTING SUBMITTED ELECTRONICALLY

An XML formatted sequence listing having the file name “108313_SequenceListing.xml” created on Oct. 11, 2023, and having a size of 133,622 bytes is filed in computer readable form concurrently with the specification. The sequence listing comprised in this XML formatted document is part of the specification and is herein incorporated by reference in its entirety.

### FIELD

Embodiments disclosed herein relate to the field of plant molecular biology, including to DNA constructs for conferring insect resistance to a plant. Embodiments disclosed herein also include insect resistant corn plant containing event DP-051291-2 and assays for detecting the presence of event DP-051291-2 in a sample and compositions thereof.

### BACKGROUND

Corn is an important crop and is a primary food source in many areas of the world. Damage caused by insect pests is a major factor in the loss of the world's corn crops, despite the use of protective measures such as chemical pesticides. In view of this, insect resistance has been genetically engineered into crops such as corn in order to control insect damage and to reduce the need for traditional chemical pesticides. One group of genes which have been utilized for the production of transgenic insect resistant crops is the delta-endotoxin group from Bacillus thuringiensis (Bt). Delta-endotoxins have been successfully expressed in crop plants such as cotton, potatoes, rice, sunflower, as well as corn, and in certain circumstances have proven to provide excellent control over insect pests. (Perlak, F. J et al. (1990) Bio Technology 8:939-943; Perlak, F. J. et al. (1993) Plant Mol. Biol. 22:313-321; Fujimoto, H. et al. (1993) Bio Technology 11:1151-1155; Tu et al. (2000) Nature Biotechnology 18:1101-1104; PCT publication WO 01/13731; and Bing, J. W. et al. (2000) Efficacy of Cry1F Transgenic Maize, 14^{th} Biennial International Plant Resistance to Insects Workshop, Fort Collins, CO).

The expression of transgenes in plants is known to be influenced by many different factors, including the orientation and composition of the cassettes driving expression of the individual genes of interest, and the location in the plant genome, perhaps due to chromatin structure (e.g., heterochromatin) or the proximity of transcriptional regulatory elements (e.g., enhancers) close to the integration site (Weising et al. (1988) Ann. Rev. Genet. 22:421-477).

It would be advantageous to be able to detect the presence of a particular event in order to determine whether progeny of a sexual cross contain a transgene of interest.

It is possible to detect the presence of a transgene by a nucleic acid detection method by, e.g., a polymerase chain reaction (PCR) or DNA hybridization using nucleic acid probes. These detection methods generally focus on frequently used genetic elements, such as promoters, terminators, marker genes, etc., because for many DNA constructs, the coding region is interchangeable. As a result, such methods may not be useful for discriminating between different events, particularly those produced using the same DNA construct or very similar constructs unless the DNA sequence of the flanking DNA adjacent to the inserted heterologous DNA is known.

### SUMMARY

The embodiments relate to the insect resistant corn (Zea mays) plant event DP-051291-2, also referred to as “maize line DP-051291-2,” “maize event DP-051291-2,” and “DP-051291-2 maize,” to the DNA plant expression construct of corn plant event DP-051291-2, and to methods and compositions for the detection of the transgene construct, flanking, and insertion (the target locus) regions in corn plant event DP-051291-2 and progeny thereof.

In one aspect compositions and methods relate to methods for producing and selecting an insect resistant monocot crop plant. Compositions include a DNA construct that when expressed in plant cells and plants confers resistance to insects. In one aspect, a DNA construct, capable of introduction into and replication in a host cell, is provided that when expressed in plant cells and plants confers insect resistance to the plant cells and plants. Maize event DP-051291-2 was produced by Agrobacterium-mediated transformation with plasmid PHP74638 (FIG. 1). As described herein, these events include the IPD072Aa (polynucleotide SEQ ID NO: 4 and amino acid SEQ ID NO: 5) cassette (Table 1), which confers resistance to certain Coleopteran plant pests. The insect control components have demonstrated efficacy against Coleopteran insect species, particularly western corn rootworm (WCR). In some embodiments, a polynucleotide encoding an IPD072Aa polypeptide comprises a sequence having at least 95%, 96%, 97%, 98%, or 99% sequence identity to SEQ ID NO: 4. In some embodiments, an IPD072Aa polypeptide comprises a sequence having 95%, 96%, 97%, 98%, or 99% sequence identity to SEQ ID NO: 5.

According to some embodiments, compositions and methods are provided for identifying a novel corn plant designated DP-051291-2 (ATCC Deposit Number PTA-127358). The methods are based on primers or probes which specifically recognize 5′ and/or 3′ flanking sequence of DP-051291-2. DNA molecules are provided that comprise primer sequences that when utilized in a PCR reaction will produce amplicons unique to the transgenic event DP-051291-2. In one embodiment, the corn plant and seed comprising these molecules is contemplated. Further, kits utilizing these primer sequences for the identification of the DP-051291-2 event are provided.

Some embodiments relate to specific flanking sequences of DP-051291-2 as described herein, which can be used to develop identification methods for DP-051291-2 in biological samples. More particularly, the disclosure relates to 5′ and/or 3′ flanking regions of DP-051291-2, which can be used for the development of specific primers and probes. Further embodiments relate to identification methods for the presence of DP-051291-2 in biological samples based on the use of such specific primers or probes.

According to some embodiments, methods of detecting the presence of DNA corresponding to the corn event DP-051291-2 in a sample are provided. Such methods comprise: (a) contacting the sample comprising DNA with a DNA primer set, that when used in a nucleic acid amplification reaction with genomic DNA extracted from corn comprising event DP-051291-2 produces an amplicon that is diagnostic for corn event DP-051291-2, respectively; (b) performing a nucleic acid amplification reaction, thereby producing the amplicon; and (c) detecting the amplicon. In some aspects, the primer set comprises SEQ ID NOs: 6 and 7, and optionally a probe comprising SEQ ID NO: 8.

According to some embodiments, methods of detecting the presence of a DNA molecule corresponding to the DP-051291-2 event in a sample comprise: (a) contacting the sample comprising DNA extracted from a corn plant with a DNA probe molecule that hybridizes under stringent hybridization conditions with DNA extracted from corn event DP-051291-2 and does not hybridize under the stringent hybridization conditions with a control corn plant DNA; (b) subjecting the sample and probe to stringent hybridization conditions; and (c) detecting hybridization of the probe to the DNA extracted from corn event DP-051291-2. More specifically, a method for detecting the presence of a DNA molecule corresponding to the DP-051291-2 event in a sample comprising (a) contacting the sample comprising DNA extracted from a corn plant with a DNA probe molecule that comprises sequences that are unique to the event, e.g. junction sequences, wherein said DNA probe molecule hybridizes under stringent hybridization conditions with DNA extracted from corn event DP-051291-2 and does not hybridize under the stringent hybridization conditions with a control corn plant DNA; (b) subjecting the sample and probe to stringent hybridization conditions; and (c) detecting hybridization of the probe to the DNA.

In addition, a kit and methods for identifying event DP-051291-2 in a biological sample which detects a DP-051291-2 specific region are provided.

DNA molecules are provided that comprise at least one junction sequence of DP-051291-2; wherein a junction sequence spans the junction located between heterologous DNA inserted into the genome and the DNA from the maize cell flanking the insertion site and may be diagnostic for the DP-051291-2 event.

According to some embodiments, methods of producing an insect resistant corn plant comprise the steps of: (a) sexually crossing a first parental corn line comprising the expression cassettes disclosed herein, which confer resistance to insects, and a second parental corn line that lacks such expression cassettes, thereby producing a plurality of progeny plants; and (b) selecting a progeny plant that is insect resistant. Such methods may optionally comprise the further step of back-crossing the progeny plant to the second parental corn line to produce a true-breeding corn plant that is insect resistant.

Some embodiments provide a method of producing a corn plant that is resistant to insects comprising transforming a corn cell with the DNA construct PHP74638, growing the transformed corn cell into a corn plant, selecting the corn plant that shows resistance to insects, and further growing the corn plant into a fertile corn plant. The fertile corn plant can be self-pollinated or crossed with compatible corn varieties to produce insect resistant progeny.

Some embodiments further relate to a DNA detection kit for identifying maize event DP-051291-2 in biological samples. The kit comprises a first primer which specifically recognizes the 5′ or 3′ flanking region of DP-051291-2, and a second primer which specifically recognizes a sequence within the non-native target locus DNA of DP-051291-2, respectively, or within the flanking DNA, for use in a PCR identification protocol. A further embodiment relates to a kit for identifying event DP-051291-2 in biological samples, which kit comprises a specific probe having a sequence which corresponds or is complementary to, a sequence having between about 80% and 100% sequence identity with a specific region of event DP-051291-2. The sequence of the probe corresponds to a specific region comprising part of the 5′ or 3′ flanking region of event DP-051291-2. In some embodiments, the first or second primer comprises any one of SEQ ID NOs: 6-7, 9-10, 12-13, 15-16, or 18-19.

The methods and kits encompassed by the embodiments disclosed herein can be used for different purposes such as, but not limited to the following: to identify event DP-051291-2 in plants, plant material or in products such as, but not limited to, food or feed products (fresh or processed) comprising, or derived from plant material; additionally or alternatively, the methods and kits can be used to identify transgenic plant material for purposes of segregation between transgenic and non-transgenic material; additionally or alternatively, the methods and kits can be used to determine the quality of plant material comprising maize event DP-051291-2. The kits may also contain the reagents and materials necessary for the performance of the detection method.

A further embodiment relates to the DP-051291-2 maize plant or its parts, including, but not limited to, pollen, ovules, vegetative cells, the nuclei of pollen cells, and the nuclei of egg cells of the corn plant DP-051291-2 and the progeny derived thereof. In another embodiment, the DNA primer molecules targeting the maize plant and seed of DP-051291-2 provide a specific amplicon product.

### DESCRIPTION OF THE DRAWINGS

FIG. 1. shows a schematic diagram of plasmid PHP74638 with genetic elements indicated. Plasmid size is 66,641 bp (SEQ ID NO: 1).

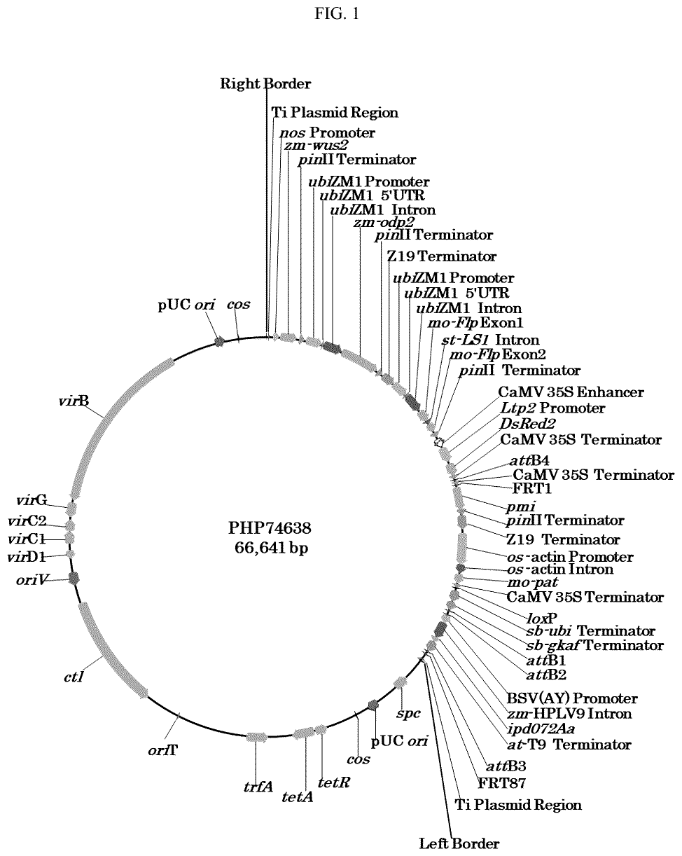

FIG. 2. shows a schematic diagram of the T-DNA region of plasmid PHP74638 indicating the recombination fragment region flanked by the FRT1 and FRT87 sites, that contains the pmi, mo-pat, and ipd072Aa gene cassettes intended for incorporation into the maize genome, and the zm-wus2, zm-odp2, mo-Flp, and DsRed2 gene cassettes not intended for incorporation into the maize genome. The size of the T-DNA is 23,712 bp (SEQ ID NO: 2). The zm-wus2 and zm-odp2 developmental genes were present to increase transformation efficiency and the tissue-specific red fluorescent protein DsRed2 gene was present to allow for differentiation during seed sorting.

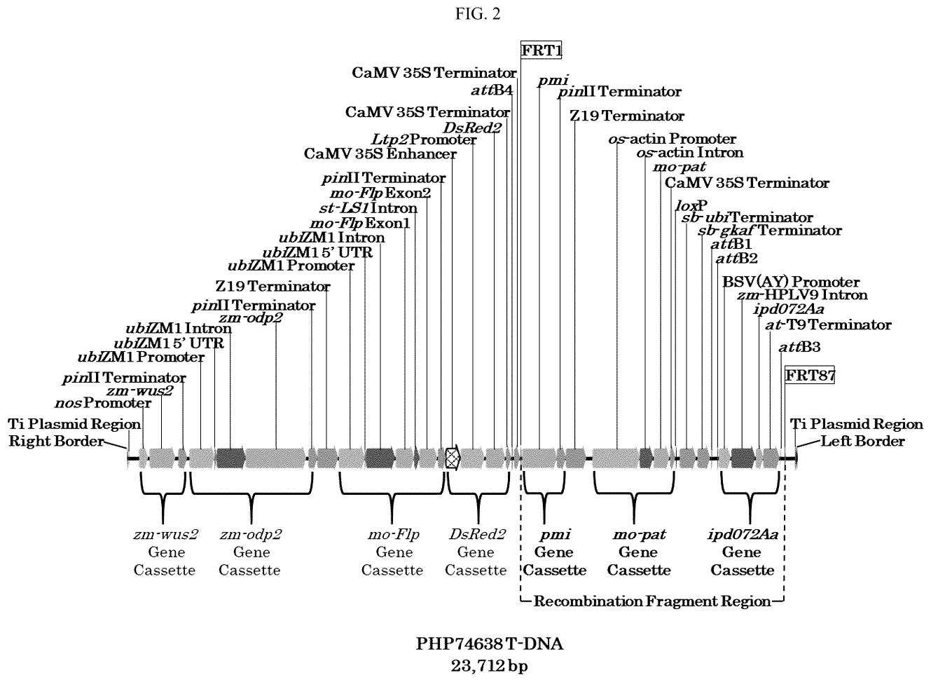

FIG. 3. shows a schematic map of the insertion in DP-051291-2 maize based on the sequencing analysis described herein. The flanking maize genome is represented by the horizontal black bars. A single copy of the insertion, derived from PHP74638 and PHP50742, is integrated into the maize genome (SEQ ID NO: 2 is the T-DNA sequence). Within the insertion, the landing pad sequences from PHP50742 and the trait genes derived from PHP74638 are indicated. SEQ ID NO: 3 is the complete insert sequence and flanking genomic regions. The FRT1 and FRT87 sites that are the targets of recombination during the SSI process are indicated.

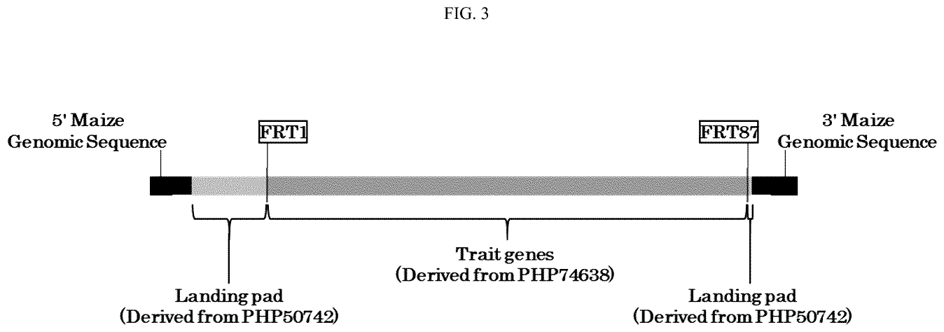

FIG. 4. shows a schematic Diagram of the Transformation and Development of DP-051291-2.

### DETAILED DESCRIPTION

As used herein the singular forms “a”, “and”, and “the” include plural referents unless the context clearly dictates otherwise. Thus, for example, reference to “a cell” includes a plurality of such cells and reference to “the protein” includes reference to one or more proteins and equivalents thereof, and so forth. All technical and scientific terms used herein have the same meaning as commonly understood to one of ordinary skill in the art to which this disclosure belongs unless clearly indicated otherwise.

Compositions of this disclosure include seed deposited as ATCC Patent Deposit No. PTA-127358 and plants, plant cells, and seed derived therefrom. Applicant(s) deposited at least 625 seeds of maize event DP-051291-2 (Patent Deposit No. PTA-127358) with the American Type Culture Collection (ATCC), Manassas, VA 20110-2209 USA, on Aug. 19, 2022. These deposits will be maintained under the terms of the Budapest Treaty on the International Recognition of the Deposit of Microorganisms for the Purposes of Patent Procedure. The seeds deposited with the ATCC on Aug. 19, 2022 were taken from the deposit maintained by Pioneer Hi-Bred International, Inc., 7250 NW 62^{nd} Avenue, Johnston, Iowa 50131-1000. Access to this deposit will be available during the pendency of the application to the Commissioner of Patents and Trademarks and persons determined by the Commissioner to be entitled thereto upon request. Upon allowance of any claims in the application, the Applicant(s) will make available to the public, pursuant to 37 C.F.R. § 1.808, sample(s) of the deposit of at least 625 seeds of hybrid maize with the American Type Culture Collection (ATCC), 10801 University Boulevard, Manassas, VA 20110-2209. This deposit of seed of maize event DP-051291-2 will be maintained in the ATCC depository, which is a public depository, for a period of 30 years, or 5 years after the most recent request, or for the enforceable life of the patent, whichever is longer, and will be replaced if it becomes nonviable during that period. Additionally, Applicant(s) have satisfied all the requirements of 37 C.F.R. §§ 1.801-1.809, including providing an indication of the viability of the sample upon deposit. Applicant(s) have no authority to waive any restrictions imposed by law on the transfer of biological material or its transportation in commerce. Applicant(s) do not waive any infringement of their rights granted under this patent or rights applicable to event DP-051291-2 under the Plant Variety Protection Act (7 USC 2321 et seq.). Unauthorized seed multiplication is prohibited. The seed may be regulated.

A single copy of the insertion derived from PHP74638 and PHP50742 was integrated into the maize genome. Within the insertion were the landing pad sequences from PHP50742 and the trait genes derived from PHP74638 containing the pmi, mo-pat, and ipd072Aa gene cassettes.

A first gene cassette (pmi gene cassette) contains the phosphomannose isomerase (pmi) gene from Escherichia coli (Negrotto et al., 2000). Expression of the PMI protein in plants serves as a selectable marker which allows plant tissue growth with mannose as the carbon source. The PMI protein is 391 amino acids in length and has a molecular weight of approximately 43 kDa. As present in the T-DNA region of PHP74638, the pmi gene lacks a promoter, but its location next to the flippase recombination target site, FRT1, allows post-recombination expression by an appropriately placed promoter. The terminator for the pmi gene is a copy of the pinII terminator. An additional Z19 terminator present is intended to prevent transcriptional interference between cassettes.

A second gene cassette (mo-pat gene cassette) contains a maize optimized version of the phosphinothricin acetyl transferase gene (mo-pat) from Streptomyces viridochromogenes (Wohlleben et al., 1988). The mo-pat gene expresses the phosphinothricin acetyl transferase (PAT) enzyme that confers tolerance to phosphinothricin. The PAT protein is 183 amino acids in length and has a molecular weight of approximately 21 kDa. Expression of the mo-pat gene is controlled by the promoter and intron region of the Oryza sativa (rice) actin (os-actin) gene (GenBank accession CP018159; GenBank accession EU155408.1), in conjunction with a third copy of the CaMV35S terminator. Two additional terminators are present to prevent transcriptional interference: the terminator regions from the Sorghum bicolor (sorghum) ubiquitin (sb-ubi) gene (Phytozome gene ID Sobic.004G049900.1; U.S. Pat. No. 9,725,731 [Abbitt, 2017]) and γ-kafarin (sb-gkaf) gene (de Freitas et al., 1994), respectively.

A third gene cassette contains the insecticidal protein gene, ipd072Aa, from Pseudomonas chlororaphis (Schellenberger et al., 2016). The expressed IPD072Aa protein in plants is effective against certain coleopteran pests. The IPD072Aa protein is 86 amino acids in length and has a molecular weight of approximately 10 kDa. Expression of the ipd072Aa gene is controlled by the promoter region from the banana streak virus of acuminata Yunnan strain [BSV(AY)](U.S. Pat. No. 8,338,662) and the intron region from the maize ortholog of a rice (Oryza sativa) hypothetical protein (zm-HPLV9), a predicted maize calmodulin 5 gene (international patent application publication WO/2016/109157 [Abbitt and Shen, 2016]), in conjunction with the terminator region from an Arabidopsis thaliana putative gene of the mannose-binding protein superfamily (at-T9) (U.S. Pat. No. 10,059,953).

As used herein, the term “corn” means Zea mays or maize and includes all plant varieties that can be bred with corn, including wild maize species.

As used herein, the terms “insect resistant” and “impacting insect pests” refers to effecting changes in insect feeding, growth, and/or behavior at any stage of development, including but not limited to: killing the insect; retarding growth; reducing reproductive capability; inhibiting feeding; and the like.

As used herein, the terms “pesticidal activity” and “insecticidal activity” are used synonymously to refer to activity of an organism or a substance (such as, for example, a protein) that can be measured by numerous parameters including, but not limited to, pest mortality, pest weight loss, pest attraction, pest repellency, and other behavioral and physical changes of a pest after feeding on and/or exposure to the organism or substance for an appropriate length of time. For example, “pesticidal proteins” are proteins that display pesticidal activity by themselves or in combination with other proteins.

As used herein, “insert DNA” refers to the heterologous DNA within the expression cassettes used to transform the plant material while “flanking DNA” can exist of either genomic DNA naturally present in an organism such as a plant, or foreign (heterologous) DNA introduced via the transformation process which is extraneous to the original insert DNA molecule, e.g. fragments associated with the transformation event. A “flanking region” or “flanking sequence” as used herein refers to a sequence of at least 10 bp (in some narrower embodiments, at least 20 bp, at least 50 bp, and up to at least 5000 bp), which is located either immediately upstream of and contiguous with and/or immediately downstream of and contiguous with the original non-native insert DNA molecule. Transformation procedures of the foreign DNA may result in transformants containing different flanking regions characteristic and unique for each transformant. When recombinant DNA is introduced into a plant through traditional crossing, its flanking regions will generally not be changed. It may be possible for single nucleotide changes to occur in the flanking regions through generations of plant breeding and traditional crossing. Transformants will also contain unique junctions between a piece of heterologous insert DNA and genomic DNA, or two (2) pieces of genomic DNA, or two (2) pieces of heterologous DNA. A “junction” is a point where two (2) specific DNA fragments join. For example, a junction exists where insert DNA joins flanking DNA. A junction point also exists in a transformed organism where two (2) DNA fragments join together in a manner that is modified from that found in the native organism. “Junction DNA” refers to DNA that comprises a junction point. Junction sequences set forth in this disclosure include a junction point located between the maize genomic DNA and the 5′ end of the insert, which range from at least −5 to +5 nucleotides of the junction point (SEQ ID NO: 26), from at least −10 to +10 nucleotides of the junction point (SEQ ID NO: 27), and from at least −25 to +25 nucleotides of the junction point (SEQ ID NO: 28); and a junction point located between the 3′ end of the insert and maize genomic DNA, which range from at least −5 to +5 nucleotides of the junction point (SEQ ID NO: 29), from at least −10 to +10 nucleotides of the junction point (SEQ ID NO: 30), and from at least −25 to +25 nucleotides of the junction point (SEQ ID NO: 31). Junction sequences set forth in this disclosure also include a junction point located between the target locus and the 5′ end of the insert. In some embodiments, SEQ ID NOs: 8 or 21 for DP-051291-2 represent the junction point located between the target locus and the 3′ end of the insert. The complete insert with flanking regions is represented in SEQ ID NO: 3. In some embodiments, the insert and flanking regions comprise a polynucleotide having at least 95%, 96%, 97%, 98%, or 99% sequence identity compared to SEQ ID NO: 3.

In one embodiment, seeds, plants, and plant parts comprising corn event DP-051291-2 are provided, wherein said seeds, plants, and plant parts comprise a DNA sequence chosen from SEQ ID NO: 26, SEQ ID NO: 27, SEQ ID NO: 28, SEQ ID NO: 29, SEQ ID NO: 30, and SEQ ID NO: 31, or a DNA sequence chosen from a sequence having at least 95% sequence identity to SEQ ID NO: 26, SEQ ID NO: 27, SEQ ID NO: 28, SEQ ID NO: 29, SEQ ID NO: 30, and SEQ ID NO: 31, wherein a representative sample of the corn event DP-051291-2 seed has been deposited with American Type Culture Collection (ATCC) with Accession No. PTA-127358. In another embodiment, seeds, plants, and plant parts comprising corn event DP-051291-2 are provided, wherein said seeds, plants, and plant parts comprise SEQ ID NO: 3 or a nucleotide sequence having at least 95% sequence identity to SEQ ID NO: 3, wherein a representative sample of the corn event DP-051291-2 seed has been deposited with American Type Culture Collection (ATCC) with Accession No. PTA-127358.

As used herein, “heterologous” in reference to a nucleic acid sequence is a nucleic acid sequence that originates from a different non-sexually compatible species, or, if from the same species, is substantially modified from its native form in composition and/or genomic locus by deliberate human intervention. For example, a promoter operably linked to a heterologous nucleotide sequence can be from a species different from that from which the nucleotide sequence was derived, or, if from the same species, the promoter is not naturally found operably linked to the nucleotide sequence. A heterologous protein may originate from a foreign species, or, if from the same species, is substantially modified from its original form by deliberate human intervention.

The term “regulatory element” refers to a nucleic acid molecule having gene regulatory activity, i.e. one that has the ability to affect the transcriptional and/or translational expression pattern of an operably linked transcribable polynucleotide. The term “gene regulatory activity” thus refers to the ability to affect the expression of an operably linked transcribable polynucleotide molecule by affecting the transcription and/or translation of that operably linked transcribable polynucleotide molecule. Gene regulatory activity may be positive and/or negative and the effect may be characterized by its temporal, spatial, developmental, tissue, environmental, physiological, pathological, cell cycle, and/or chemically responsive qualities as well as by quantitative or qualitative indications.

“Promoter” refers to a nucleotide sequence capable of controlling the expression of a coding sequence or functional RNA. In general, a coding sequence is located 3′ to a promoter sequence. The promoter sequence comprises proximal and more distal upstream elements, the latter elements are often referred to as enhancers. Accordingly, an “enhancer” is a nucleotide sequence that can stimulate promoter activity and may be an innate element of the promoter or a heterologous element inserted to enhance the level or tissue-specificity of a promoter. Promoters may be derived in their entirety from a native gene or be composed of different elements derived from different promoters found in nature, or even comprise synthetic nucleotide segments. It is understood by those skilled in the art that different regulatory elements may direct the expression of a gene in different tissues or cell types, or at different stages of development, or in response to different environmental conditions. Promoters that cause a nucleic acid fragment to be expressed in most cell types at most times are commonly referred to as “constitutive promoters”. It is further recognized that since in most cases the exact boundaries of regulatory sequences have not been completely defined, nucleic acid fragments of different lengths may have identical or similar promoter activity.

The “translation leader sequence” refers to a nucleotide sequence located between the promoter sequence of a gene and the coding sequence. The translation leader sequence is present in the fully processed mRNA upstream of the translation start sequence. The translation leader sequence may affect numerous parameters including, processing of the primary transcript to mRNA, mRNA stability and/or translation efficiency.

The “3′ non-coding sequences” refer to nucleotide sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

A DNA construct is an assembly of DNA molecules linked together that provide one or more expression cassettes. The DNA construct may be a plasmid that is enabled for self-replication in a bacterial cell and contains various endonuclease enzyme restriction sites that are useful for introducing DNA molecules that provide functional genetic elements, i.e., promoters, introns, leaders, coding sequences, 3′ termination regions, among others; or a DNA construct may be a linear assembly of DNA molecules, such as an expression cassette. The expression cassette contained within a DNA construct comprises the necessary genetic elements to provide transcription of a messenger RNA. The expression cassette can be designed to express in prokaryotic cells or eukaryotic cells. Expression cassettes of the embodiments are designed to express in plant cells.

The DNA molecules disclosed herein are provided in expression cassettes for expression in an organism of interest. The cassette includes 5′ and 3′ regulatory sequences operably linked to a coding sequence. “Operably linked” means that the nucleic acid sequences being linked are contiguous and, where necessary to join two protein coding regions, contiguous and in the same reading frame. Operably linked is intended to indicate a functional linkage between a promoter and a second sequence, wherein the promoter sequence initiates and mediates transcription of the DNA sequence corresponding to the second sequence. The cassette may additionally contain at least one additional gene to be co-transformed into the organism. Alternatively, the additional gene(s) can be provided on multiple expression cassettes or multiple DNA constructs.

The expression cassette may include in the 5′ to 3′ direction of transcription: a transcriptional and translational initiation region, a coding region, and a transcriptional and translational termination region functional in the organism serving as a host. The transcriptional initiation region (e.g., the promoter) may be native or analogous, or foreign or heterologous to the host organism. Additionally, the promoter may be the natural sequence or alternatively a synthetic sequence. The expression cassettes may additionally contain 5′ leader sequences in the expression cassette construct. Such leader sequences can act to enhance translation.

It is to be understood that as used herein the term “transgenic” generally includes any cell, cell line, callus, tissue, plant part, or plant, the genotype of which has been altered by the presence of a heterologous nucleic acid including those initially so altered as well as those created by sexual crosses or asexual propagation from the initial transgenic and retains such heterologous nucleic acids.

A transgenic “event” is produced by transformation of plant cells with a heterologous DNA construct(s), including a nucleic acid expression cassette that comprises a transgene of interest, the regeneration of a population of plants resulting from the insertion of the transgene into the genome of the plant, and selection of a particular plant characterized by insertion into a particular genome location. An event is characterized phenotypically by the expression of the transgene. At the genetic level, an event is part of the genetic makeup of a plant. The term “event” also refers to progeny produced by a sexual outcross between the transformant and another variety, wherein the progeny includes the heterologous DNA. After back-crossing to a recurrent parent, the inserted DNA and the linked flanking genomic DNA from the transformed parent is present in the progeny of the cross at the same chromosomal location. A progeny plant may contain sequence changes to the insert arising as a result of conventional breeding techniques. The term “event” also refers to DNA from the original transformant comprising the inserted DNA and flanking sequence immediately adjacent to the inserted DNA that would be expected to be transferred to a progeny that receives inserted DNA including the transgene of interest as the result of a sexual cross of one parental line that includes the inserted DNA (e.g., the original transformant and progeny resulting from selfing) and a parental line that does not contain the inserted DNA.

An insect resistant DP-051291-2 corn plant may be bred by first sexually crossing a first parental corn plant having the transgenic DP-051291-2 event plant and progeny thereof derived from transformation with the expression cassettes of the embodiments that confers insect resistance, and a second parental corn plant that lacks such expression cassettes, thereby producing a plurality of first progeny plants; and then selecting a first progeny plant that is resistant to insects; and selfing the first progeny plant, thereby producing a plurality of second progeny plants; and then selecting from the second progeny plants an insect resistant plant. These steps can further include the back-crossing of the first insect resistant progeny plant or the second insect resistant progeny plant to the second parental corn plant or a third parental corn plant, thereby producing a corn plant that is resistant to insects. The term “selfing” refers to self-pollination, including the union of gametes and/or nuclei from the same organism.

As used herein, the term “plant” includes reference to whole plants, parts of plants, plant organs (e.g., leaves, stems, roots, etc.), seeds, plant cells, and progeny of same. In some embodiments, parts of transgenic plants comprise, for example, plant cells, protoplasts, tissues, callus, embryos as well as flowers, stems, fruits, leaves, and roots originating in transgenic plants or their progeny previously transformed with a DNA molecule disclosed herein, and therefore consisting at least in part of transgenic cells.

As used herein, the term “plant cell” includes, without limitation, seeds, suspension cultures, embryos, meristematic regions, callus tissue, leaves, roots, shoots, gametophytes, sporophytes, pollen, and microspores. The class of plants that may be used is generally as broad as the class of higher plants amenable to transformation techniques, including both monocotyledonous and dicotyledonous plants.

“Transformation” refers to the transfer of a nucleic acid fragment into the genome of a host organism, resulting in genetically stable inheritance. Host plants containing the transformed nucleic acid fragments are referred to as “transgenic” plants.

As used herein, the term “progeny,” in the context of event DP-051291-2, denotes an offspring of any generation of a parent plant which comprises corn event DP-051291-2.

Isolated polynucleotides disclosed herein may be incorporated into recombinant constructs, typically DNA constructs, which are capable of introduction into and replication in a host cell. Such a construct may be a vector that includes a replication system and sequences that are capable of transcription and translation of a polypeptide-encoding sequence in a given host cell. A number of vectors suitable for stable transfection of plant cells or for the establishment of transgenic plants have been described in, e.g., Pouwels et al., (1985; Supp. 1987) Cloning Vectors: A Laboratory Manual, Weissbach and Weissbach (1989) Methods for Plant Molecular Biology, (Academic Press, New York); and Flevin et al., (1990) Plant Molecular Biology Manual, (Kluwer Academic Publishers). Typically, plant expression vectors include, for example, one or more cloned plant genes under the transcriptional control of 5′ and 3′ regulatory sequences and a dominant selectable marker. Such plant expression vectors also can contain a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal.

During the process of introducing an insert into the genome of plant cells, it is not uncommon for some deletions or other alterations of the insert and/or genomic flanking sequences to occur. Thus, the relevant segment of the plasmid sequence provided herein might comprise some minor variations including truncations. The same is possible for the flanking sequences and junction sequences provided herein. Thus, a plant comprising a polynucleotide having some range of identity with the subject flanking and/or insert sequences is within the scope of the subject disclosure. Identity to the sequence of the present disclosure may be a polynucleotide sequence having at least 65% sequence identity, at least 70% sequence identity, at least 75% sequence identity at least 80% identity, or at least 85%, 86%, 87%, 88%, 89%, 90%, 91%, 92%, 93%, 94%, 95%, 96%, 97%, 98%, 99% sequence identity with a sequence exemplified or described herein. Hybridization and hybridization conditions as provided herein can also be used to define such plants and polynucleotide sequences of the subject disclosure. A sequence comprising the flanking sequences plus the full insert sequence can be confirmed with reference to the deposited seed.

In some embodiments, two different transgenic plants can also be crossed to produce offspring that contain two independently segregating added, exogenous genes. Selfing of appropriate progeny can produce plants that are homozygous for both added, exogenous genes. Back-crossing to a parental plant and out-crossing with a non-transgenic plant are also contemplated, as is vegetative propagation.

A “probe” is an isolated nucleic acid to which is attached a conventional, synthetic detectable label or reporter molecule, e.g., a radioactive isotope, ligand, chemiluminescent agent, or enzyme. Such a probe is complementary to a strand of a target nucleic acid, for example, to a strand of isolated DNA from corn event DP-051291-2 whether from a corn plant or from a sample that includes DNA from the event. Probes may include not only deoxyribonucleic or ribonucleic acids but also polyamides and other modified nucleotides that bind specifically to a target DNA sequence and can be used to detect the presence of that target DNA sequence.

“Primers” are isolated nucleic acids that anneal to a complementary target DNA strand by nucleic acid hybridization to form a hybrid between the primer and the target DNA strand, then extended along the target DNA strand by a polymerase, e.g., a DNA polymerase. Primer pairs refer to their use for amplification of a target nucleic acid sequence, e.g., by PCR or other conventional nucleic-acid amplification methods. “PCR” or “polymerase chain reaction” is a technique used for the amplification of specific DNA segments (see, U.S. Pat. Nos. 4,683,195 and 4,800,159; herein incorporated by reference).

Probes and primers are of sufficient nucleotide length to bind to the target DNA sequence specifically in the hybridization conditions or reaction conditions determined by the operator. This length may be of any length that is of sufficient length to be useful in a detection method of choice. Generally, 11 nucleotides or more in length, 18 nucleotides or more, and 22 nucleotides or more, are used. Such probes and primers hybridize specifically to a target sequence under high stringency hybridization conditions. Probes and primers according to embodiments may have complete DNA sequence similarity of contiguous nucleotides with the target sequence, although probes differing from the target DNA sequence and that retain the ability to hybridize to target DNA sequences may be designed by conventional methods. Probes can be used as primers, but are generally designed to bind to the target DNA or RNA and are not used in an amplification process.

Specific primers may be used to amplify an integration fragment to produce an amplicon that can be used as a “specific probe” for identifying event DP-051291-2 in biological samples. When the probe is hybridized with the nucleic acids of a biological sample under conditions which allow for the binding of the probe to the sample, this binding can be detected and thus allow for an indication of the presence of event DP-051291-2 in the biological sample. In an embodiment of the disclosure, the specific probe is a sequence which, under appropriate conditions, hybridizes specifically to a region within the 5′ or 3′ flanking region of the event and also comprises a part of the foreign DNA contiguous therewith. The specific probe may comprise a sequence of at least 80%, from 80 and 85%, from 85 and 90%, from 90 and 95%, and from 95 and 100% identical (or complementary) to a specific region of the event.

Methods for preparing and using probes and primers are described, for example, in Sambrook et al., Molecular Cloning: A Laboratory Manual, 2^{nd} ed., vol. 1-3, Cold Spring Harbor Laboratory Press, Cold Spring Harbor, N.Y. 1989 (hereinafter, “Sambrook et al., 1989”); Ausubel et al. eds., Current Protocols in Molecular Biology, Greene Publishing and Wiley-Interscience, New York, 1995 (with periodic updates) (hereinafter, “Ausubel et al., 1995”); and Innis et al., PCR Protocols: A Guide to Methods and Applications, Academic Press: San Diego, 1990. PCR primer pairs can be derived from a known sequence, for example, by using computer programs intended for that purpose such as the PCR primer analysis tool in Vector NTI version 6 (Informax Inc., Bethesda MD); PrimerSelect (DNASTAR Inc., Madison, WI); and Primer (Version 0.5©, 1991, Whitehead Institute for Biomedical Research, Cambridge, Mass.). Additionally, the sequence can be visually scanned and primers manually identified using guidelines known to one of skill in the art.

A “kit” as used herein refers to a set of reagents, and optionally instructions, for the purpose of performing method embodiments of the disclosure, more particularly, the identification of event DP-051291-2 in biological samples. A kit may be used, and its components can be specifically adjusted, for purposes of quality control (e.g. purity of seed lots), detection of event DP-051291-2 in plant material, or material comprising or derived from plant material, such as but not limited to food or feed products. “Plant material” as used herein refers to material which is obtained or derived from a plant.

Primers and probes based on the flanking DNA and insert sequences disclosed herein can be used to confirm (and, if necessary, to correct) the disclosed sequences by conventional methods, e.g., by re-cloning and sequencing such sequences. The nucleic acid probes and primers hybridize under stringent conditions to a target DNA sequence. Any conventional nucleic acid hybridization or amplification method may be used to identify the presence of DNA from a transgenic event in a sample.

A nucleic acid molecule is said to be the “complement” of another nucleic acid molecule if they exhibit complete complementarity or minimal complementarity. As used herein, molecules are said to exhibit “complete complementarity” when every nucleotide of one of the molecules is complementary to a nucleotide of the other. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Conventional stringency conditions are described by Sambrook et al., 1989, and by Haymes et al., In: Nucleic Acid Hybridization, a Practical Approach, IRL Press, Washington, D.C. (1985), departures from complete complementarity are therefore permissible, as long as such departures do not completely preclude the capacity of the molecules to form a double-stranded structure. In order for a nucleic acid molecule to serve as a primer or probe it need only be sufficiently complementary in sequence to be able to form a stable double-stranded structure under the particular solvent and salt concentrations employed.

In hybridization reactions, specificity is typically the function of post-hybridization washes, the critical factors being the ionic strength and temperature of the final wash solution. The thermal melting point (Tₘ) is the temperature (under defined ionic strength and pH) at which 50% of a complementary target sequence hybridizes to a perfectly matched probe. For DNA-DNA hybrids, the Tₘ can be approximated from the equation of Meinkoth and Wahl (1984) Anal. Biochem. 138:267-284: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the Tₘ for the specific sequence and its complement at a defined ionic strength and pH. However, in some embodiments, other stringency conditions can be applied, including severely stringent conditions can utilize a hybridization and/or wash at 1, 2, 3, or 4° C. lower than the Tₘ; moderately stringent conditions can utilize a hybridization and/or wash at 6, 7, 8, 9, or 10° C. lower than the Tₘ; low stringency conditions can utilize a hybridization and/or wash at 11, 12, 13, 14, 15, or 20° C. lower than the Tₘ.

Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), a user may choose to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, New York); and Ausubel et al., eds. (1995) and Sambrook et al. (1989).

In some embodiments, a complementary sequence has the same length as the nucleic acid molecule to which it hybridizes. In some embodiments, the complementary sequence is 1, 2, 3, 4, 5, 6, 7, 8, 9, or 10 nucleotides longer or shorter than the nucleic acid molecule to which it hybridizes. In some embodiments, the complementary sequence is 1%, 2%, 3%, 4%, or 5% longer or shorter than the nucleic acid molecule to which it hybridizes. In some embodiments, a complementary sequence is complementary on a nucleotide-for-nucleotide basis, meaning that there are no mismatched nucleotides (each A pairs with a T and each G pairs with a C). In some embodiments, a complementary sequence comprises 1, 2, 3, 4, 5, 6, 7, 8, 9, 10 or less mismatches. In some embodiments, the complementary sequence comprises 1%, 2%, 3%, 4%, 5%, 6%, 7%, 8%, 9%, or 10% or less mismatches.

“Percent (%) sequence identity” with respect to a reference sequence (subject) is determined as the percentage of amino acid residues or nucleotides in a candidate sequence (query) that are identical with the respective amino acid residues or nucleotides in the reference sequence, after aligning the sequences and introducing gaps, if necessary, to achieve the maximum percent sequence identity, and not considering any amino acid conservative substitutions as part of the sequence identity. Alignment for purposes of determining percent sequence identity can be achieved in various ways that are within the skill in the art, for instance, using publicly available computer software such as BLAST, BLAST-2. Those skilled in the art can determine appropriate parameters for aligning sequences, including any algorithms needed to achieve maximal alignment over the full length of the sequences being compared. The percent identity between the two sequences is a function of the number of identical positions shared by the sequences (e.g., percent identity of query sequence=number of identical positions between query and subject sequences/total number of positions of query sequence×100).

Regarding the amplification of a target nucleic acid sequence (e.g., by PCR) using a particular amplification primer pair, stringent conditions permit the primer pair to hybridize only to the target nucleic-acid sequence to which a primer having the corresponding wild-type sequence (or its complement) would bind and optionally to produce a unique amplification product, the amplicon, in a DNA thermal amplification reaction.

As used herein, “amplified DNA” or “amplicon” refers to the product of nucleic acid amplification of a target nucleic acid sequence that is part of a nucleic acid template. For example, to determine whether a corn plant resulting from a sexual cross contains transgenic event genomic DNA from the corn plant disclosed herein, DNA extracted from a tissue sample of a corn plant may be subjected to a nucleic acid amplification method using a DNA primer pair that includes a first primer derived from flanking sequence adjacent to the insertion site of inserted heterologous DNA, and a second primer derived from the inserted heterologous DNA to produce an amplicon that is diagnostic for the presence of the event DNA. Alternatively, the second primer may be derived from the flanking sequence. The amplicon is of a length and has a sequence that is also diagnostic for the event. The amplicon may range in length from the combined length of the primer pairs plus one nucleotide base pair to any length of amplicon producible by a DNA amplification protocol. Alternatively, primer pairs can be derived from flanking sequence on both sides of the inserted DNA so as to produce an amplicon that includes the entire insert nucleotide sequence of the PHP74638 expression construct as well as a portion of the sequence flanking the transgenic insert. A member of a primer pair derived from the flanking sequence may be located a distance from the inserted DNA sequence, this distance can range from one nucleotide base pair up to the limits of the amplification reaction. The use of the term “amplicon” specifically excludes primer dimers that may be formed in the DNA thermal amplification reaction.

Nucleic acid amplification can be accomplished by any of the various nucleic acid amplification methods known in the art, including PCR. A variety of amplification methods are known in the art and are described, inter alia, in U.S. Pat. Nos. 4,683,195 and 4,683,202 and in Innis et al., (1990) supra. PCR amplification methods have been developed to amplify up to 22 Kb of genomic DNA and up to 42 Kb of bacteriophage DNA (Cheng et al., Proc. Natl. Acad. Sci. USA 91:5695-5699, 1994). These methods as well as other methods known in the art of DNA amplification may be used in the practice of the embodiments of the present disclosure. It is understood that a number of parameters in a specific PCR protocol may need to be adjusted to specific laboratory conditions and may be slightly modified and yet allow for the collection of similar results. These adjustments will be apparent to a person skilled in the art.

The amplicon produced by these methods may be detected by a plurality of techniques, including, but not limited to, Genetic Bit Analysis (Nikiforov, et al. Nucleic Acid Res. 22:4167-4175, 1994) where a DNA oligonucleotide is designed which overlaps both the adjacent flanking DNA sequence and the inserted DNA sequence. The oligonucleotide is immobilized in wells of a microwell plate. Following PCR of the region of interest (for example, using one primer in the inserted sequence and one in the adjacent flanking sequence) a single-stranded PCR product can be hybridized to the immobilized oligonucleotide and serve as a template for a single base extension reaction using a DNA polymerase and labeled ddNTPs specific for the expected next base. Readout may be fluorescent or ELISA-based. A signal indicates presence of the insert/flanking sequence due to successful amplification, hybridization, and single base extension.

Another detection method is the pyrosequencing technique as described by Winge (2000) Innov. Pharma. Tech. 00:18-24. In this method an oligonucleotide is designed that overlaps the adjacent DNA and insert DNA junction. The oligonucleotide is hybridized to a single-stranded PCR product from the region of interest (for example, one primer in the inserted sequence and one in the flanking sequence) and incubated in the presence of a DNA polymerase, ATP, sulfurylase, luciferase, apyrase, adenosine 5′ phosphosulfate and luciferin. dNTPs are added individually and the incorporation results in a light signal which is measured. A light signal indicates the presence of the transgene insert/flanking sequence due to successful amplification, hybridization, and single or multi-base extension.

Fluorescence polarization as described by Chen et al., (1999) Genome Res. 9:492-498 is also a method that can be used to detect an amplicon. Using this method an oligonucleotide is designed which overlaps the flanking and inserted DNA junction. The oligonucleotide is hybridized to a single-stranded PCR product from the region of interest (for example, one primer in the inserted DNA and one in the flanking DNA sequence) and incubated in the presence of a DNA polymerase and a fluorescent-labeled ddNTP. Single base extension results in incorporation of the ddNTP. Incorporation can be measured as a change in polarization using a fluorometer. A change in polarization indicates the presence of the transgene insert/flanking sequence due to successful amplification, hybridization, and single base extension.

Quantitative PCR (qPCR) is described as a method of detecting and quantifying the presence of a DNA sequence and is fully understood in the instructions provided by commercially available manufacturers. Briefly, in one such qPCR method, a FRET oligonucleotide probe is designed which overlaps the flanking and insert DNA junction. The FRET probe and PCR primers (one primer in the insert DNA sequence and one in the flanking genomic sequence) are cycled in the presence of a thermostable polymerase and dNTPs. Hybridization of the FRET probe results in cleavage and release of the fluorescent moiety away from the quenching moiety on the FRET probe. A fluorescent signal indicates the presence of the flanking/transgene insert sequence due to successful amplification and hybridization.

Molecular beacons have been described for use in sequence detection as described in Tyangi et al. (1996) Nature Biotech. 14:303-308. Briefly, a FRET oligonucleotide probe is designed that overlaps the flanking and insert DNA junction. The unique structure of the FRET probe results in it containing secondary structure that keeps the fluorescent and quenching moieties in close proximity. The FRET probe and PCR primers (for example, one primer in the insert DNA sequence and one in the flanking sequence) are cycled in the presence of a thermostable polymerase and dNTPs. Following successful PCR amplification, hybridization of the FRET probe to the target sequence results in the removal of the probe secondary structure and spatial separation of the fluorescent and quenching moieties. A fluorescent signal results. A fluorescent signal indicates the presence of the flanking/transgene insert sequence due to successful amplification and hybridization.

A hybridization reaction using a probe specific to a sequence found within the amplicon is yet another method used to detect the amplicon produced by a PCR reaction.

Insect pests include insects selected from the orders Coleoptera, Diptera, Hymenoptera, Lepidoptera, Mallophaga, Homoptera, Hemiptera, Orthoptera, Thysanoptera, Dermaptera, Isoptera, Anoplura, Siphonaptera, Trichoptera, etc., particularly Coleoptera.

Of interest are larvae and adults of the order Coleoptera including weevils from the families Anthribidae, Bruchidae, and Curculionidae including, but not limited to: Anthonomus grandis Boheman (boll weevil); Cylindrocopturus adspersus LeConte (sunflower stem weevil); Diaprepes abbreviatus Linnaeus (Diaprepes root weevil); Hypera punctata Fabricius (clover leaf weevil); Lissorhoptrus oryzophilus Kuschel (rice water weevil); Metamasius hemipterus hemipterus Linnaeus (West Indian cane weevil); M. hemipterus sericeus Olivier (silky cane weevil); Sitophilus granarius Linnaeus (granary weevil); S. oryzae Linnaeus (rice weevil); Smicronyx fulvus LeConte (red sunflower seed weevil); S. sordidus LeConte (gray sunflower seed weevil); Sphenophorus maidis Chittenden (maize billbug); S. livis Vaurie (sugarcane weevil); Rhabdoscelus obscurus Boisduval (New Guinea sugarcane weevil); flea beetles, cucumber beetles, rootworms, leaf beetles, potato beetles, and leafminers in the family Chrysomelidae including, but not limited to: Chaetocnema ectypa Horn (desert corn flea beetle); C. pulicaria Melsheimer (corn flea beetle); Colaspis brunnea Fabricius (grape colaspis); Diabrotica barberi Smith & Lawrence (northern corn rootworm); D. undecimpunctata howardi Barber (southern corn rootworm); D. virgifera virgifera LeConte (western corn rootworm); Leptinotarsa decemlineata Say (Colorado potato beetle); Oulema melanopus Linnaeus (cereal leaf beetle); Phyllotreta cruciferae Goeze (corn flea beetle); Zygogramma exclamationis Fabricius (sunflower beetle); beetles from the family Coccinellidae including, but not limited to: Epilachna varivestis Mulsant (Mexican bean beetle); chafers and other beetles from the family Scarabaeidae including, but not limited to: Antitrogus parvulus Britton (Childers cane grub); Cyclocephala borealis Arrow (northern masked chafer, white grub); C. immaculata Olivier (southern masked chafer, white grub); Dermolepida albohirtum Waterhouse (Greyback cane beetle); Euetheola humilis rugiceps LeConte (sugarcane beetle); Lepidiota frenchi Blackburn (French's cane grub); Tomarus gibbosus De Geer (carrot beetle); T. subtropicus Blatchley (sugarcane grub); Phyllophaga crinita Burmeister (white grub); P. latifrons LeConte (June beetle); Popillia japonica Newman (Japanese beetle); Rhizotrogus majalis Razoumowsky (European chafer); carpet beetles from the family Dermestidae; wireworms from the family Elateridae, Eleodes spp., Melanotus spp. including M. communis Gyllenhal (wireworm); Conoderus spp.; Limonius spp.; Agriotes spp.; Ctenicera spp.; Aeolus spp.; bark beetles from the family Scolytidae; beetles from the family Tenebrionidae; beetles from the family Cerambycidae such as, but not limited to, Migdolus fryanus Westwood (longhorn beetle); and beetles from the Buprestidae family including, but not limited to, Aphanisticus cochinchinae seminulum Obenberger (leaf-mining buprestid beetle).

In some embodiments the DP-051291-2 maize event may further comprise a stack of additional traits. Plants comprising stacks of polynucleotide sequences can be obtained by either or both of traditional breeding methods or through genetic engineering methods. These methods include, but are not limited to, breeding individual lines each comprising a polynucleotide of interest, transforming a transgenic plant comprising a gene disclosed herein with a subsequent gene and co-transformation of genes into a single plant cell. As used herein, the term “stacked” includes having the multiple traits present in the same plant (i.e., both traits are incorporated into the nuclear genome, one trait is incorporated into the nuclear genome and one trait is incorporated into the genome of a plastid or both traits are incorporated into the genome of a plastid).

In some embodiments the DP-051291-2 maize event disclosed herein, alone or stacked with one or more additional insect resistance traits can be stacked with one or more additional input traits (e.g., herbicide resistance, fungal resistance, virus resistance, stress tolerance, disease resistance, male sterility, stalk strength, and the like) or output traits (e.g., increased yield, modified starches, improved oil profile, balanced amino acids, high lysine or methionine, increased digestibility, improved fiber quality, drought resistance, and the like). Thus, the embodiments can be used to provide a complete agronomic package of improved crop quality with the ability to flexibly and cost effectively control any number of agronomic pests.

In a further embodiment, the DP-051291-2 maize event may be stacked with one or more additional insecticidal toxins, including, but not limited to, a Cry3B toxin disclosed in U.S. Pat. Nos. 8,101,826, 6,551,962, 6,586,365, 6,593,273, and PCT Publication WO 2000/011185; a mCry3B toxin disclosed in U.S. Pat. Nos. 8,269,069, and 8,513,492; a mCry3A toxin disclosed in U.S. Pat. Nos. 8,269,069, 7,276,583 and 8,759,620; or a Cry34/35 toxin disclosed in U.S. Pat. Nos. 7,309,785, 7,524,810, 7,985,893, 7,939,651 and 6,548,291. In a further embodiment, the DP-051291-2 maize event may be stacked with one or more additional transgenic events containing these Bt insecticidal toxins and other Coleopteran active Bt insecticidal traits for example, event MON863 disclosed in U.S. Pat. No. 7,705,216; event MIR604 disclosed in U.S. Pat. No. 8,884,102; event 5307 disclosed in U.S. Pat. No. 9,133,474; event DAS-59122 disclosed in U.S. Pat. No. 7,875,429; event DP-4114 disclosed in U.S. Pat. No. 8,575,434; event MON 87411 disclosed in U.S. Pat. No. 9,441,240; event DP-23211 disclosed in International Patent Application Publication Number WO 2019/209700; and event MON88017 disclosed in U.S. Pat. No. 8,686,230 all of which are incorporated herein by reference. In some embodiments, the DP-051291-2 maize event may be stacked with MON-87429-9 (MON87429 Event); MON87403; MON95379; MON95275; MON87427; MON87419; MON-00603-6 (NK603); MON-87460-4; LY038; DAS-06275-8; BT176; BT11; MIR162; GA21; MZDTO9Y; SYN-05307-1; DP-915635-2; DP-23211; DP-910521-2; DAS-01131-3; and DAS-40278-9.

In a further embodiment, the DP-051291-2 maize event may be stacked with one or more additional of the following provided herbicidal tolerance traits. The glyphosate herbicide contains a mode of action by inhibiting the EPSPS enzyme (5-enolpyruvylshikimate-3-phosphate synthase). This enzyme is involved in the biosynthesis of aromatic amino acids that are essential for growth and development of plants. Various enzymatic mechanisms are known in the art that can be utilized to inhibit this enzyme. The genes that encode such enzymes can be operably linked to the gene regulatory elements of the subject disclosure. In an embodiment, selectable marker genes include, but are not limited to genes encoding glyphosate resistance genes include: mutant EPSPS genes such as 2mEPSPS genes, cp4 EPSPS genes, mEPSPS genes, aroA genes; and glyphosate degradation genes such as glyphosate acetyl transferase genes (gat) and glyphosate oxidase genes (gox). These traits are currently marketed as Gly-Tol™, Optimum@GAT®, Agrisure® GT and Roundup Ready®. Resistance genes for glufosinate and/or bialaphos compounds include dsm-2, bar and pat genes. The bar andpat traits are currently marketed as LibertyLink®. Also included are tolerance genes that provide resistance to 2,4-D such as aad-1 genes (it should be noted that aad-1 genes have further activity on arloxyphenoxypropionate herbicides) and aad-12 genes (it should be noted that aad-12 genes have further activity on pyidyloxyacetate synthetic auxins). These traits are marketed as Enlist® crop protection technology. Resistance genes for ALS inhibitors (sulfonylureas, imidazolinones, triazolopyrimidines, pyrimidinylthiobenzoates, and sulfonylamino-carbonyl-triazolinones) are known in the art.

These resistance genes most commonly result from point mutations to the ALS encoding gene sequence. Other ALS inhibitor resistance genes include hra genes, the csr1-2 genes, Sr-HrA genes, and surB genes. Some of the traits are marketed under the tradename Clearfield®. Herbicides that inhibit HPPD include the pyrazolones such as pyrazoxyfen, benzofenap, and topramezone; triketones such as mesotrione, sulcotrione, tembotrione, benzobicyclon; and diketonitriles such as isoxaflutole. These exemplary HPPD herbicides can be tolerated by known traits. Examples of HPPD inhibitors include hppdPF_W336 genes (for resistance to isoxaflutole) and avhppd-03 genes (for resistance to meostrione). An example of oxynil herbicide tolerant traits include the bxn gene, which has been showed to impart resistance to the herbicide/antibiotic bromoxynil. Resistance genes for dicamba include the dicamba monooxygenase gene (dmo) as disclosed in International PCT Publication No. WO 2008/105890. Resistance genes for PPO or PROTOX inhibitor type herbicides (e.g., acifluorfen, butafenacil, flupropazil, pentoxazone, carfentrazone, fluazolate, pyraflufen, aclonifen, azafenidin, flumioxazin, flumiclorac, bifenox, oxyfluorfen, lactofen, fomesafen, fluoroglycofen, and sulfentrazone) are known in the art. Exemplary genes conferring resistance to PPO include over expression of a wild-type Arabidopsis thaliana PPO enzyme (Lermontova I and Grimm B, (2000) Overexpression of plastidic protoporphyrinogen IX oxidase leads to resistance to the diphenyl-ether herbicide acifluorfen. Plant Physiol 122:75-83.), the B. subtilis PPO gene (Li, X. and Nicholl D. 2005. Development of PPO inhibitor-resistant cultures and crops. Pest Manag. Sci. 61:277-285 and Choi K W, Han O, Lee H J, Yun Y C, Moon Y H, Kim M K, Kuk Y I, Han S U and Guh J O, (1998) Generation of resistance to the diphenyl ether herbicide, oxyfluorfen, via expression of the Bacillus subtilis protoporphyrinogen oxidase gene in transgenic tobacco plants. Biosci Biotechnol Biochem 62:558-560.) Resistance genes for pyridinoxy or phenoxy proprionic acids and cyclohexones include the ACCase inhibitor-encoding genes (e.g., Acc1-S1, Acc1-S2 and Acc1-S3). Exemplary genes conferring resistance to cyclohexanediones and/or aryloxyphenoxypropanoic acid include haloxyfop, diclofop, fenoxyprop, fluazifop, and quizalofop. Finally, herbicides can inhibit photosynthesis, including triazine or benzonitrile are provided tolerance by psbA genes (tolerance to triazine), Is+ genes (tolerance to triazine), and nitrilase genes (tolerance to benzonitrile). The above list of herbicide tolerance genes is not meant to be limiting. Any herbicide tolerance genes are encompassed by the present disclosure.

In some embodiments, the disclosed compositions can be introduced into the genome of a plant using genome editing technologies, or previously introduced polynucleotides in the genome of a plant may be edited using genome editing technologies. For example, the disclosed polynucleotides can be introduced into a desired location in the genome of a plant through the use of genome editing systems such as TALENs, meganucleases, zinc finger nucleases, CRISPR-Cas, and the like. For example, the disclosed polynucleotides can be introduced into a desired location in a genome using a CRISPR-Cas system, for the purpose of site-specific insertion. The desired location in a plant genome can be any desired target site for insertion, such as a genomic region amenable for breeding or may be a target site located in a genomic window with existing trait(s) of interest. Existing trait(s) of interest could be either endogenous traits or previously introduced traits.

In some embodiments, where the disclosed polynucleotide has previously been introduced into a genome, genome editing or genome engineering technologies may be used to alter or modify the introduced polynucleotide sequence, including flanking chromosomal genomic sequences. Site specific modifications that can be introduced into the disclosed compositions include those produced using any method for introducing site specific modification, including, but not limited to, through the use of sequence repair oligonucleotides alone, or through the use of site-directed genome modification tools such as TALENs, meganucleases, zinc finger nucleases, CRISPR-Cas, and the like, with or without donor DNA. Site specific modifications to the disclosed polynucleotides (including genomic flanking and junction sequences) may include, but are not limited to, changes to codon usage, changes to regulatory elements such as promoters, introns, terminators, enhancers, 5′ or 3′ untranslated regions (UTRs), or other noncoding sequences, and other regions of the polynucleotide, where the modifications do not adversely affect the phenotypic characteristics of the resulting maize plant. DP-051291-2 event plants containing modified polynucleotide sequences are also contemplated herein.

Cas polypeptides suitable for introducing site-specific modifications include, for example, Cas9, Cas12f (Cas-alpha, Cas14), Cas12l (Cas-beta), Cas12a (Cpf1), Cas12b (a C2c1 protein), Cas13 (a C2c2 protein), Cas12c (a C2c3 protein), Cas12d, Cas12e, Cas12g, Cas12h, Cas12i, Cas12j, Cas12k, Cas3, Cas3-HD, Cas 5, Cas6, Cas7, Cas8, Cas10, or combinations or complexes of these. In some aspects, transposon-associated TnpB, a programmable RNA-guided DNA endonuclease can be used.

In some aspects, a genome editing system comprises a Cas-alpha (e.g., Cas12f) endonuclease and one or more guide polynucleotides that introduce one or more site-specific modifications in a target polynucleotide sequence, resulting in a modified target sequence. As used herein, “altered target site”, “altered target sequence”, “modified target site,” and “modified target sequence” are used interchangeably and refer to a target sequence as disclosed herein that comprises at least one alteration or modification when compared to a non-altered target sequence. Such alterations or modifications include, for example: (i) replacement or substitution of at least one nucleotide, (ii) deletion of at least one nucleotide, (iii) insertion of at least one nucleotide, or (iv) any combination of (i)-(iii).

In some aspects, a genome editing system comprises a Cas-alpha endonuclease, one or more guide polynucleotides, and optionally a donor DNA. Some exemplary Cas-alpha endonucleases are described, for example, in WO2020123887.

In some aspects, a genome editing system comprises a Cas polypeptide, one or more guide polynucleotides, and optionally donor DNA, and editing a target polynucleotide sequence comprises nonhomologous end-joining (NHEJ) or homologous recombination (HR) following a Cas polypeptide-mediated double-strand break. Once a double-strand break is induced in the DNA, the cell's DNA repair mechanism is activated to repair the break. The most common repair mechanism to bring the broken ends together is the nonhomologous end-joining pathway (Bleuyard et al., (2006) DNA Repair 5:1-12). As a result, deletions, insertions, or other rearrangements are possible (Siebert and Puchta, (2002) Plant Cell 14:1121-31; Pacher et al., (2007) Genetics 175:21-9). Alternatively, the double-strand break can be repaired by homologous recombination between homologous DNA sequences. Once the sequence around the double-strand break is altered, for example, by exonuclease activities involved in the maturation of double-strand breaks, gene conversion pathways can restore the original structure if a homologous sequence is available, such as a homologous chromosome in non-dividing somatic cells, or a sister chromatid after DNA replication (Molinier et al., (2004) Plant Cell 16:342-52). Ectopic and/or epigenic DNA sequences may also serve as a DNA repair template for homologous recombination (Puchta, (1999) Genetics 152:1173-81).

In some aspects, the genome editing system comprises a Cas polypeptide, one or more guide polynucleotides, and a donor DNA. As used herein, “donor DNA” is a DNA construct that comprises a polynucleotide of interest to be inserted into the genomic target site of a Cas polypeptide. Once a double-strand break is introduced in the target site by the endonuclease, the first and second regions of homology of the donor DNA can undergo homologous recombination with their corresponding genomic regions of homology resulting in exchange of DNA between the donor DNA and the target genomic region. As such, the provided methods result in the integration of the polynucleotide of interest of the donor DNA into the double-strand break in the target site in the plant genome, thereby altering the original target site and producing an altered genomic target site.

In some aspects, a genome editing system comprises a base editing agent and a plurality of guide polynucleotides and editing a target polynucleotide sequence comprises introducing a plurality of nucleobase edits in the target polynucleotide sequence resulting in a variant nucleotide sequence. Other aspects include modified DP-051291-2 event plants produced using a genome editing system.

One or more nucleobases of a target genomic sequence can be chemically altered, in some cases to change the base from one type to another, for example from a Cytosine to a Thymine, or an Adenine to a Guanine. In some aspects, a plurality of bases, for example 2 or more, 5 or more, 10 or more, 20 or more, 30 or more, 40 or more, 50 or more, 60 or more, 70 or more, 80 or more 90 or more, 100 or more, or even greater than 100, 200 or more, up to thousands of bases may be modified or altered, to produce a plant with a plurality of modified bases.

Any base editing complex, such as a base editing agent associated with an RNA-guided polypeptide (such as e.g., dCas associated with a deaminase), may be used to target and bind to a desired locus in the genome of an organism and chemically modify one or more nucleotides of a target genomic sequence.

Site-specific nucleotide base conversions can be achieved to engineer one or more nucleotide changes to create one or more edits into the genome. These include for example, a site-specific base edit mediated by a C·G to T·A or an A·T to G·C base editing deaminase enzymes (Gaudelli et al., Programmable base editing of A·T to G·C in genomic DNA without DNA cleavage.” Nature (2017); Nishida et al. “Targeted nucleotide editing using hybrid prokaryotic and vertebrate adaptive immune systems.” Science 353 (6305) (2016); Komor et al. “Programmable editing of a target base in genomic DNA without double-stranded DNA cleavage.” Nature 533 (7603) (2016):420-4. A catalytically “dead” or inactive Cas (dCas) polypeptide, for example an inactive Cas9 (dCas9), Cas12f (dCas12f), or another Cas polypeptide disclosed herein, fused to a cytidine deaminase or an adenine deaminase protein becomes a specific base editor that can alter DNA bases without inducing a DNA break. Base editors convert C->T (or G->A on the opposite strand) or an adenine base editor that would convert adenine to inosine, resulting in an A->G change within an editing window specified by the guide polynucleotides. Any molecule that effects a change in a nucleobase is a “base editing agent”. The dCas forms a functional complex with a guide polynucleotide that shares homology with a genomic sequence at the target site, and is further complexed with the deaminase molecule. The guided Cas polypeptide recognizes and binds to a target sequence, opening the double-strand to expose individual bases. In the case of a cytidine deaminase, the deaminase deaminates the cytosine base and creates a uracil. Uracil glycosylase inhibitor (UGI) is provided to prevent the conversion of U back to C. DNA replication or repair mechanisms then convert the Uracil to a thymine (U to T), and subsequent repair of the opposing base (formerly G in the original G-C pair) to an Adenine, creating a T-A pair.

One or more nucleotides of the inserted event and/or the flanking genomic DNA can be modified using a prime editing technology. See e.g., Anzalone et al., Search-and-replace genome editing without double-strand breaks or donor DNA. Nature 576, 149-157 (2019). A prime editing complex includes a prime editing protein that contains an RNA-guided DNA-nicking domain, such as a Cas nickase (e.g., Cas9 nickase, Casd12f1 nickase), fused to a reverse transcriptase domain and complexed with a pegRNA. The PE-pegRNA complex is able to introduce targeted DNA edits at desired locations in the genome, by binding the target DNA and nicking the PAM-containing strand. The resulting 3′ end hybridizes to a chosen primer binding site and then primes reverse transcription of new DNA sequence containing the desired edit using the reverse transcriptase template of the pegRNA.

The resulting regulatory expression elements of the disclosed recombinant expression cassette(s) may be truncated or may include a polynucleotide sequence having at least 65% sequence identity, at least 70% sequence identity, at least 75% sequence identity at least 80% identity, or at least 85%, 86%, 87%, 88%, 89%, 90%, 91%, 92%, 93%, 94%, 95%, 96%, 97%, 98%, 99% sequence identity with a regulatory element sequence exemplified or described herein.

Other modifications may include modifications to other portions of the DNA of the DP-051291-2 event. In some embodiments, genome engineering technologies can be used to relocate one or more expression cassettes described herein to one or more different locations of the same chromosome, or different chromosomes of maize or a different crop. In such embodiment, polynucleotides comprising one or more of the junction sequences described herein (SEQ ID NOs: 26 and/or 29) may be retained with the expression cassette(s), either partially or fully, or may be removed. Furthermore, genomic flanking sequence(s) described herein may also be retained with the expression cassette(s), either partially or fully, or may be removed.

In another embodiment, genome engineering technologies may be used to co-locate one or more transgene(s) or expression cassette(s) in physical proximity to the 5′ or 3′ junction sequence(s) described herein. With regard to physical position on a chromosome, co-located transgenes and/or expression cassettes can be separated from the 5′ or 3′ junction sequence(s), e.g., by about 1 megabase (MB; 1 million nucleotides), about 500 kilobases (Kb; 1000 nucleotides), about 400 Kb, about 300 Kb, about 200 Kb, about 100 Kb, about 50 Kb, about 25 Kb, about 10 Kb, about 5 Kb, about 4 Kb, about 3 Kb, about 2 Kb, about 1 Kb, about 500 nucleotides, about 250 nucleotides, or less. With regard to genetic distance on a chromosome, co-located transgenes and/or expression cassettes can be separated from the 5′ or 3′ junction sequence(s), e.g., by about 10 cM, 9, 8, 7, 6, 5, 4, 3, 2, 1, 0.75, 0.5, 0.25, 0.1 cM. For example, one or more of the expression cassette(s) obtained from one or more of the additional transgenic events described above may be co-located in physical proximity to the 5′ or 3′ junction sequence(s) described herein.

In another embodiment, polynucleotides comprising one of the junction sequences (SEQ ID NOs: 26 or 29) may be introduced at either or both ends of the inserted heterologous DNA. For example, a polynucleotide comprising the 5′ junction sequence may be deleted and replaced with a polynucleotide comprising the 3′ junction sequence, or vice versa.

In another embodiment, genome editing technologies may be used to modify the previously introduced polynucleotide(s) by inverting at least one of the polynucleotide(s) of the inserted DNA of the DP-051291-2 event.

Such genome editing technologies can be used to modify the previously introduced polynucleotide through the insertion, deletion, and/or substitution of one or more nucleotides within the introduced polynucleotide. Alternatively, double-stranded break technologies can be used to add additional nucleotide sequences to the introduced polynucleotide. Additional sequences that may be added include, but are not limited to, additional expression elements, such as enhancer and promoter sequences. Sequences that may be deleted include, but are not limited to, regulatory elements or portions thereof that when deleted do not adversely affect function. Modifications to modulate expression patterns (e.g., reducing the expression level of the insecticidal polypeptide in certain tissue) is also contemplated by site-directed modification to the introduced expression cassette.

In another embodiment, genome engineering technologies may be used to delete or modify all or part of one or more expression cassette(s) of the DP-051291-2 event as deposited with the ATCC on Aug. 19, 2022 having accession number PTA-127358. In this embodiment, the resulting maize plant derived from the DP-051291-2 event as deposited with the ATCC on Aug. 19, 2022 having accession number PTA-127358 may comprise a portion of the expression cassette(s) described herein, none of the expression cassette(s) described herein, or modifications of the expression cassette(s) described herein.

In another embodiment, targeted DSB technologies may be used to position additional insecticidally-active proteins in close proximity to the disclosed compositions disclosed herein within the genome of a plant, in order to generate molecular stacks of insecticidally-active proteins.

In another embodiment, the polynucleotide sequences disclosed herein are used in a method comprising designing guide polynucleotides, such as guide RNAs (gRNAs), that recognize said polynucleotide sequences, synthesizing or obtaining said guide polynucleotides, and introducing said guide polynucleotides as part of genome engineering compositions to modify the DNA of the DP-051291-2 event as deposited with the ATCC on Aug. 19, 2022 having accession number PTA-127358. Such resulting modifications may include a polynucleotide sequence having at least 65% sequence identity, at least 70% sequence identity, at least 75% sequence identity at least 80% identity, or at least 85%, 86%, 87%, 88%, 89%, 90%, 91%, 92%, 93%, 94%, 95%, 96%, 97%, 98%, 99% sequence identity with a sequence exemplified or described herein.

Embodiments include modified DP-051291-2 event plants produced using genome engineering technologies described herein.

One embodiment includes a corn plant comprising the genotype of the corn event DP-051291-2, wherein said genotype comprises a nucleotide sequence as set forth in SEQ ID NO: 26 and SEQ ID NO: 29, or a nucleotide sequence having at least 90%, 91%, 92%, 93%, 94%, 95%, 96%, 97%, 98%, or 99% sequence identity to SEQ ID NO: 26 and SEQ ID NO: 29.

Another embodiment includes the corn plant comprising the genotype of the corn event DP-051291-2 of any prior embodiment, wherein said genotype comprises the nucleotide sequence set forth in SEQ ID NO: 27 and SEQ ID NO: 30, or a nucleotide sequence having at least 90%, 91%, 92%, 93%, 94%, 95%, 96%, 97%, 98%, or 99% sequence identity to SEQ ID NO: 27 and SEQ ID NO: 30.

Another embodiment includes the corn plant comprising the genotype of the corn event DP-051291-2 of any prior embodiment, wherein said genotype comprises the nucleotide sequence set forth in SEQ ID NO: 28 and SEQ ID NO: 31, or a nucleotide sequence having at least 90%, 91%, 92%, 93%, 94%, 95%, 96%, 97%, 98%, or 99% sequence identity to SEQ ID NO: 28 and SEQ ID NO: 31.

One embodiment includes a DNA construct comprising operably linked expression cassettes, wherein one of the expression cassettes comprises:
1) a BSV(AY) Promoter;
2) a zm-HPLV9 Intron;
3) an ipd072Aa; and
4) an at-T9 Terminator.

Another embodiment includes a plant comprising the DNA construct comprising two operably linked expression cassettes of any prior embodiment.

A further embodiment includes the plant comprising the DNA construct comprising two operably linked expression cassettes of any prior embodiment, wherein said plant is a corn plant.

One embodiment includes a plant comprising the sequence set forth in SEQ ID NO: 21, or a sequence having at least 95% sequence identity to SEQ ID NO: 21.

One embodiment includes a corn event DP-051291-2, wherein a representative sample of seed of said corn event has been deposited with American Type Culture Collection (ATCC) with Accession No. PTA-127358.

Other embodiments include plant parts of the corn event DP-051291-2, wherein a representative sample of seed of said corn event has been deposited with American Type Culture Collection (ATCC) with Accession No. PTA-127358 of any prior embodiment.

One embodiment includes seed comprising corn event DP-051291-2, wherein said seed comprises a DNA molecule chosen from SEQ ID NO: 26 and SEQ ID NO: 29, wherein a representative sample of the corn event DP-051291-2 seed has been deposited with American Type Culture Collection (ATCC) with Accession No. PTA-127358.

Another embodiment includes a corn plant, or part thereof, grown from the seed comprising corn event DP-051291-2, wherein said seed comprises a DNA molecule chosen from SEQ ID NO: 26 and SEQ ID NO: 29, wherein a representative sample of the corn event DP-051291-2 seed has been deposited with American Type Culture Collection (ATCC) with Accession No. PTA-127358 of any prior embodiment.

A further embodiment includes a transgenic seed produced from the corn plant of a corn event DP-051291-2, wherein a representative sample of seed of said corn event has been deposited with American Type Culture Collection (ATCC) with Accession No. PTA-127358 of any prior embodiment.

Other embodiments include a transgenic corn plant, or part thereof, grown from the seed produced from the corn plant of a corn event DP-051291-2, wherein a representative sample of seed of said corn event has been deposited with American Type Culture Collection (ATCC) with Accession No. PTA-127358 of any prior embodiment.

One embodiment includes an isolated nucleic acid molecule comprising a nucleotide sequence chosen from SEQ ID NOs: 21, and 26-31, and full length complements thereof.

One embodiment includes an amplicon comprising the nucleic acid sequence chosen from SEQ ID NOs: 21-25 and full length complements thereof.

One embodiment includes a biological sample or extract derived from corn event DP-051291-2 plant, tissue, or seed, wherein said sample or extract comprises a nucleotide sequence which is or is complementary to a sequence chosen from SEQ ID NO: 26 and SEQ ID NO: 29, wherein said nucleotide sequence is detectable in said sample or extract using a nucleic acid amplification or nucleic acid hybridization method, wherein a representative sample of said corn event DP-051291-2 seed has been deposited with American Type Culture Collection (ATCC) with Accession No. PTA-127358.

Another embodiment includes the biological sample or extract derived from corn event DP-051291-2 plant, tissue, or seed, wherein said sample or extract comprises a nucleotide sequence which is or is complementary to a sequence chosen from SEQ ID NO: 26 and SEQ ID NO: 29, wherein said nucleotide sequence is detectable in said sample or extract using a nucleic acid amplification or nucleic acid hybridization method, wherein a representative sample of said corn event DP-051291-2 seed has been deposited with American Type Culture Collection (ATCC) with Accession No. PTA-127358 of any prior embodiment, wherein said biological sample or extract comprises plant, plant tissue, or seed of transgenic corn event DP-051291-2.

Another embodiment includes the biological sample or extract derived from corn event DP-051291-2 plant, tissue, or seed, wherein said sample or extract comprises a nucleotide sequence which is or is complementary to a sequence chosen from SEQ ID NO: 26 and SEQ ID NO: 29, wherein said nucleotide sequence is detectable in said sample or extract using a nucleic acid amplification or nucleic acid hybridization method, wherein a representative sample of said corn event DP-051291-2 seed has been deposited with American Type Culture Collection (ATCC) with Accession No. PTA-127358 of any prior embodiment, wherein said biological sample or extract is a DNA sample extracted from the transgenic corn plant event DP-051291-2, and wherein said DNA sample comprises one or more of the nucleotide sequences chosen from SEQ ID NOs: 21-31, and the complement thereof.

Another embodiment includes the biological sample or extract derived from corn event DP-051291-2 plant, tissue, or seed, wherein said sample or extract comprises a nucleotide sequence which is or is complementary to a sequence chosen from SEQ ID NO: 26 and SEQ ID NO: 29, wherein said nucleotide sequence is detectable in said sample or extract using a nucleic acid amplification or nucleic acid hybridization method, wherein a representative sample of said corn event DP-051291-2 seed has been deposited with American Type Culture Collection (ATCC) with Accession No. PTA-127358 of any prior embodiment, wherein said biological sample or extract is chosen from corn flour, corn meal, corn syrup, corn oil, corn starch, and cereals manufactured in whole or in part to contain corn by-products.

One embodiment includes a method of producing hybrid corn seeds comprising:
a) sexually crossing a first inbred corn line comprising a nucleotide chosen from SEQ ID NOs: 21-31 and a second inbred line having a different genotype;
b) growing progeny from said crossing; and
c) harvesting the hybrid seed produced thereby.

Another embodiment includes the method of producing hybrid corn seeds of any prior embodiment, wherein the first inbred corn line is a female parent or a male parent.

One embodiment includes a method for producing a corn plant resistant to coleopteran pests comprising:
a) sexually crossing a first parent corn plant with a second parent corn plant, wherein said first or second parent corn plant comprises event DP-051291-2 thereby producing a plurality of first generation progeny plants;
b) selfing the first generation progeny plant, thereby producing a plurality of second generation progeny plants; and
c) selecting from the second generation progeny plants that comprise the event DP-051291-2 and are resistant to a coleopteran pest.

Another embodiment includes a method of producing hybrid corn seeds comprising:
a) sexually crossing a first inbred corn line comprising the DNA construct comprising two operably linked expression cassettes of any prior embodiment with a second inbred line not comprising the DNA construct comprising two operably linked expression cassettes of any prior embodiment; and
b) harvesting the hybrid seed produced thereby.

Another embodiment includes the method for producing a corn plant resistant to coleopteran pests of any prior embodiment, further comprising the step of backcrossing a second generation progeny plant that comprises corn event DP-051291-2 to the parent plant that lacks the corn event DP-051291-2 DNA, thereby producing a backcross progeny plant that is resistant to a coleopteran pest.

One embodiment includes a method for producing a corn plant resistant to corn rootworm comprising:
a) crossing a first parent corn plant with a second parent corn plant, wherein said first or second parent corn plant comprises event DP-051291-2 thereby producing a plurality of first generation progeny plants;
b) selecting a first generation progeny plant that comprises the event DP-051291-2;
c) backcrossing the first generation progeny plant of step (b) with a parent plant that lacks the corn event DP-051291-2 DNA, thereby producing a plurality of backcross progeny plants; and
d) selecting from the backcross progeny plants, a plant that comprises the event DP-051291-2;
   wherein the selected backcross progeny plant of step (d) comprises SEQ ID NO: 21, 26, or 29.

Another embodiment includes the method for producing a corn plant resistant to corn rootworm of any prior embodiment, wherein the plants of the first parent corn line are the female or male parents.

Another embodiment includes hybrid seed produced by the method for producing a corn plant resistant to corn rootworm of any prior embodiment.

One embodiment includes a method of determining zygosity of a corn plant comprising event DP-051291-2 in a biological sample comprising:
a) contacting said sample with a first pair of DNA molecules and a second distinct pair of DNA molecules such that:
   1) when used in a nucleic acid amplification reaction comprising corn event DP-051291-2 DNA, produces a first amplicon that is diagnostic for event DP-051291-2, and
   2) when used in a nucleic acid amplification reaction comprising corn genomic DNA other than DP-051291-2 DNA, produces a second amplicon that is diagnostic for corn genomic DNA other than DP-051291-2 DNA;
b) performing a nucleic acid amplification reaction; and
c) detecting the amplicons so produced, wherein detection of the presence of both amplicons indicates that said sample is heterozygous for corn event DP-051291-2 DNA, wherein detection of only the first amplicon indicates that said sample is homozygous for corn event DP-051291-2 DNA.

Another embodiment includes the method of determining zygosity of a corn plant comprising event DP-051291-2 in a biological sample in any prior embodiment, wherein the first pair of DNA molecules comprises primer pair SEQ ID NOs: 6 and 7.

Another embodiment includes the method of determining zygosity of a corn plant comprising event DP-051291-2 in a biological sample in any prior embodiment, wherein the first and second pair of DNA molecules comprise a detectable label.

A further embodiment includes the method of determining zygosity of a corn plant comprising event DP-051291-2 in a biological sample, wherein the first and second pair of DNA molecules comprise a detectable label in any prior embodiment, wherein the detectable label is a fluorescent label.

Another embodiment includes the method of determining zygosity of a corn plant comprising event DP-051291-2 in a biological sample, wherein the first and second pair of DNA molecules comprise a detectable label in any prior embodiment, wherein the detectable label is covalently associated with one or more of the primer molecules.

One embodiment includes a method of detecting the presence of a nucleic acid molecule that is unique to event DP-051291-2 in a sample comprising corn nucleic acids, the method comprising:
a) contacting the sample with a pair of primers, that, when used in a nucleic-acid amplification reaction with genomic DNA from event DP-051291-2 produces an amplicon that is diagnostic for event DP-051291-2;
b) performing a nucleic acid amplification reaction, thereby producing the amplicon that is diagnostic for event DP-051291-2; and
c) detecting the amplicon that is diagnostic for event DP-051291-2.

Another embodiment includes the method of detecting the presence of a nucleic acid molecule that is unique to event DP-051291-2 in a sample comprising corn nucleic acids of any prior embodiment, wherein the nucleic acid molecule that is diagnostic for event DP-051291-2 is an amplicon produced by the nucleic acid amplification chain reaction.

Another embodiment includes the method of detecting the presence of a nucleic acid molecule that is unique to event DP-051291-2 in a sample comprising corn nucleic acids of any prior embodiment, wherein the method further comprises contacting the sample with a probe.

A further embodiment includes the method of detecting the presence of a nucleic acid molecule that is unique to event DP-051291-2 in a sample comprising corn nucleic acids further comprising contacting the sample with a probe of any prior embodiment, wherein the probe comprises a detectable label.

A further embodiment includes the method of detecting the presence of a nucleic acid molecule that is unique to event DP-051291-2 in a sample comprising corn nucleic acids further comprising contacting the sample with a probe, wherein the probe comprises a detectable label of any prior embodiment, wherein the detectable label is a fluorescent label.

A further embodiment includes the method of detecting the presence of a nucleic acid molecule that is unique to event DP-051291-2 in a sample comprising corn nucleic acids further comprising contacting the sample with a probe, wherein the probe comprises a detectable label of any prior embodiment, wherein the detectable label is covalently associated with the probe.

One embodiment includes a plurality of polynucleotide primers comprising one or more polynucleotides which target event DP-051291-2 DNA template in a sample to produce an amplicon diagnostic for event DP-051291-2 as a result of a polymerase chain reaction method.

Another embodiment includes a plurality of polynucleotide primers according to any prior embodiment, wherein
a) a first polynucleotide primer comprises a nucleotide sequence as set forth in SEQ ID NO: 6, and the complements thereof; and
b) a second polynucleotide primer comprises a nucleotide sequence as set forth in SEQ ID NO: 7, and the complements thereof.

Another embodiment includes the primers of any prior embodiment, wherein said first primer and said second primer are at least 18 nucleotides.

One embodiment includes a method of detecting the presence of DNA corresponding to event DP-051291-2 in a sample, the method comprising:
a) contacting the sample comprising maize DNA with a polynucleotide probe that hybridizes under stringent hybridization conditions with DNA from maize event DP-051291-2 and does not hybridize under said stringent hybridization conditions with a non-DP-051291-2 maize plant DNA;
b) subjecting the sample and probe to stringent hybridization conditions; and
c) detecting hybridization of the probe to the DNA;
   wherein detection of hybridization indicates the presence of event DP-051291-2.

One embodiment includes a kit for detecting nucleic acids that are unique to event DP-051291-2 comprising at least one nucleic acid molecule of sufficient length of contiguous polynucleotides to function as a primer or probe in a nucleic acid detection method, and which upon amplification of or hybridization to a target nucleic acid sequence in a sample followed by detection of the amplicon or hybridization to the target sequence, are diagnostic for the presence of nucleic acid sequences unique to event DP-051291-2 in the sample.

Another embodiment includes the kit for detecting nucleic acids that are unique to event DP-051291-2 comprising at least one nucleic acid molecule of sufficient length of contiguous polynucleotides to function as a primer or probe in a nucleic acid detection method, and which upon amplification of or hybridization to a target nucleic acid sequence in a sample followed by detection of the amplicon or hybridization to the target sequence, are diagnostic for the presence of nucleic acid sequences unique to event DP-051291-2 in the sample of any prior embodiment, wherein the nucleic acid molecule comprises a nucleotide sequence from SEQ ID NOs: 6-31.

Another embodiment includes the kit for detecting nucleic acids that are unique to event DP-051291-2 comprising at least one nucleic acid molecule of sufficient length of contiguous polynucleotides to function as a primer or probe in a nucleic acid detection method, and which upon amplification of or hybridization to a target nucleic acid sequence in a sample followed by detection of the amplicon or hybridization to the target sequence, are diagnostic for the presence of nucleic acid sequences unique to event DP-051291-2 in the sample of any prior embodiment, wherein the nucleic acid molecule is a primer chosen from SEQ ID NOs: 6-31, and the complements thereof.

Another embodiment includes the corn plant comprising the genotype of the corn event DP-051291-2 of any prior embodiment, wherein the genotype comprises a nucleotide sequence having 1, 2, 3, 4, or 5 nucleotide changes in one or more of SEQ ID NOs: 26-28, SEQ ID NOs: 29-31, or SEQ ID NO: 3.

Another embodiment includes the corn plant comprising the genotype of the corn event DP-051291-2 of any prior embodiment, further comprising the nucleotide sequence set forth in SEQ ID NO: 3, or a nucleotide sequence having at least 90%, 91%, 92%, 93%, 94%, 95%, 96%, 97%, 98%, or 99% sequence identity to the nucleotide sequence of SEQ ID NO: 3.

One embodiment includes a method of modifying the DP-051291-2 corn event, wherein a representative sample of seed of said corn event was deposited with the ATCC with accession number PTA-127358, comprising applying genome engineering technology to a DNA sequence of said DP-051291-2 corn event to modify the DNA of said corn event.

Another embodiment includes the method of modifying the DP-051291-2 corn event, wherein a representative sample of seed of said corn event was deposited with the ATCC with accession number PTA-127358, comprising applying genome engineering technology to a DNA sequence of said DP-051291-2 corn event to modify the DNA of said corn event of any prior embodiment, comprising modifying the DNA of said DP-051291-2 corn event to produce a modified DNA sequence having at least 90%, 91%, 92%, 93%, 94%, 95%, 96%, 97%, 98%, or 99% sequence identity to SEQ ID NO: 3.

Another embodiment includes the method of modifying the DP-051291-2 corn event, wherein a representative sample of seed of said corn event was deposited with the ATCC with accession number PTA-127358, comprising applying genome engineering technology to a DNA sequence of said DP-051291-2 corn event to modify the DNA of said corn event of any prior embodiment, comprising modifying the DNA of said DP-051291-2 corn event to produce a modified DNA sequence having all or a portion of SEQ ID NO: 26 or SEQ ID NO: 29 duplicated in said modified DNA sequence.

Another embodiment includes the method of modifying the DP-051291-2 corn event, wherein a representative sample of seed of said corn event was deposited with the ATCC with accession number PTA-127358, comprising applying genome engineering technology to a DNA sequence of said DP-051291-2 corn event to modify the DNA of said corn event of any prior embodiment, comprising modifying the DNA of said DP-051291-2 corn event to produce a modified DNA sequence comprising an excision from SEQ ID NO: 3.

A further embodiment includes the method of modifying the DP-051291-2 corn event, wherein a representative sample of seed of said corn event was deposited with the ATCC with accession number PTA-127358, comprising applying genome engineering technology to a DNA sequence of said DP-051291-2 corn event to modify the DNA of said corn event, comprising modifying the DNA of said DP-051291-2 corn event to produce a modified DNA sequence comprising an excision from SEQ ID NO: 3 of any prior embodiment, wherein said excision comprises an excision from one or more regulatory elements of SEQ ID NO: 3 that does not substantially affect the activity of said one or more regulatory elements.

Another embodiment includes the method of modifying the DP-051291-2 corn event, wherein a representative sample of seed of said corn event was deposited with the ATCC with accession number PTA-127358, comprising applying genome engineering technology to a DNA sequence of said DP-051291-2 corn event to modify the DNA of said corn event, comprising modifying the DNA of said DP-051291-2 corn event to produce a modified DNA sequence comprising an excision from SEQ ID NO: 3 of any prior embodiment, comprising modifying the DNA of said DP-051291-2 corn event to produce a modified DNA sequence having all or a portion of SEQ ID NO: 26 or SEQ ID NO: 29 excised from said modified DNA sequence.

Another embodiment includes the method of modifying the DP-051291-2 corn event, wherein a representative sample of seed of said corn event was deposited with the ATCC with accession number PTA-127358, comprising applying genome engineering technology to a DNA sequence of said DP-051291-2 corn event to modify the DNA of said corn event, comprising modifying the DNA of said DP-051291-2 corn event to produce a modified DNA sequence comprising an excision from SEQ ID NO: 3 of any prior embodiment, comprising modifying the DNA of said DP-051291-2 corn event to produce a modified DNA sequence having at least 30% of SEQ ID NO: 3 excised from said modified DNA sequence.

A further embodiment includes the method of modifying the DP-051291-2 corn event, wherein a representative sample of seed of said corn event was deposited with the ATCC with accession number PTA-127358, comprising applying genome engineering technology to a DNA sequence of said DP-051291-2 corn event to modify the DNA of said corn event, comprising modifying the DNA of said DP-051291-2 corn event to produce a modified DNA sequence comprising an excision from SEQ ID NO: 3 of any prior embodiment, wherein at least 80% of SEQ ID NO: 3 is excised from said modified DNA sequence.

Another embodiment includes the method of modifying the DP-051291-2 corn event, wherein a representative sample of seed of said corn event was deposited with the ATCC with accession number PTA-127358, comprising applying genome engineering technology to a DNA sequence of said DP-051291-2 corn event to modify the DNA of said corn event, comprising modifying the DNA of said DP-051291-2 corn event to produce a modified DNA sequence comprising an excision from SEQ ID NO: 3 of any prior embodiment, wherein all of SEQ ID NO: 3 is excised from said modified DNA sequence.

One embodiment includes a method of generating guide polynucleotides for use with a DP-051291-2 corn event genome editing system comprising designing one or more guide polynucleotides that recognize at least a portion of SEQ ID NO: 3 and synthesizing said guide polynucleotides.

Another embodiment includes a method of modifying the DNA of the DP-051291-2 event having accession number PTA-127358 comprising introducing said one or more guide polynucleotides for use with a DP-051291-2 corn event genome editing system of any prior embodiment as part of a genome engineering composition to a DNA of the DP-051291-2 event to modify the DNA of the DP-051291-2 event.

One embodiment includes a DP-051291-2 corn event genome editing system comprising a CAS polypeptide, one or more guide polynucleotides, and DP-051291-2 corn event donor DNA.

One embodiment includes a method of modifying at least one expression cassette of the DP-051291-2 event as deposited with the ATCC having accession number PTA-127358, wherein the method comprises using genome editing technologies to modify at least one expression cassette, wherein the resulting maize plant derived from the DP-051291-2 event comprises at least one modified cassette.

Another embodiment includes the method of modifying at least one expression cassette of the DP-051291-2 event as deposited with the ATCC having accession number PTA-127358, wherein the method comprises using genome editing technologies to modify at least one expression cassette, wherein the resulting maize plant derived from the DP-051291-2 event comprises at least one modified cassette of any prior embodiment, wherein the method comprises altering expression of IPD072Aa.

One embodiment includes a method of controlling Coleopteran insects, comprising exposing the Coleopteran insects to insect resistant maize plants of event DP-051291-2.

Another embodiment includes the method of controlling Coleopteran insects, comprising exposing the Coleopteran insects to insect resistant maize plants of event DP-051291-2 of any prior embodiment, wherein the Coleopteran insect is Corn Rootworm.

Another embodiment includes the method of controlling Coleopteran insects, comprising exposing the Coleopteran insects to insect resistant maize plants of event DP-051291-2 of any prior embodiment, wherein the damage from the Coleopteran insect is controlled for maize roots from event DP-051291-2.

Another embodiment includes a method of producing a commodity plant product comprising processing grain produced from a corn event DP-051291-2 plant comprising a nucleotide sequence which is or is complementary to a sequence chosen from SEQ ID NO: 26 and SEQ ID NO: 29, wherein a representative sample of said corn event DP-051291-2 seed has been deposited with American Type Culture Collection (ATCC) with Accession No. PTA-127358, wherein said grain is processed into a commodity plant product chosen from corn flour, corn meal, corn syrup, corn oil, corn starch, and cereals manufactured in whole or in part to contain corn by-products, wherein said commodity plant product comprises a detectable amount of said nucleotide sequence.

In some embodiments, a corn plant comprising a DP-051291-2 event may be treated with a seed treatment. In some embodiments, the seed treatment may be a fungicide, an insecticide, or an herbicide.

The following examples are offered by way of illustration and not by way of limitation.

### EXAMPLES

The following Examples are included to more fully describe embodiments of the development of maize event DP-051291-2, which resulted from the construction of 84 different construct designs tested in multiple transformation vectors to create 217 distinct vectors and the production of about 7,112 events containing IPD072 as the single insect control gene (both early stage and late-stage testing) over the course of approximately six years. Thousands of individual plants (inbred and hybrid) were analyzed, many of which were tested over numerous years across diverse environments using one or more criteria including but not limited to molecular testing, protein expression, efficacy testing, agronomic testing, and/or field testing, for the creation and selection of maize event DP-051291-2. Efficacy was primarily determined by analysis of corn rootworm node-injury score (CRWNIS), including at high pressure locations.

Trait discovery and characterization included 2,711 TO plants which generated 6,285 events over the course of approximately six years, each containing IPD072 as the single insect control gene, including 4,557 late-stage events originating from 21 distinct vectors. First year field trials included 71 commercial track events. Of this pool, five were advanced to second year field trials and two were further advanced to third year field trials. Following the third year of testing, maize event DP-051291-2 was selected.

### Example 1. Cassette Design for Transgenic Plants Containing Constructs Encoding IPD072Aa

Cassette design for IPD072Aa expression used in the molecular stacks to generate commercial track events was chosen based upon efficacy and expression in gene testing transformation experiments. A large number of different regulatory (promoters, introns) and other elements (terminators) were evaluated in gene testing experiments. The large number of different regulatory elements were used to evaluate expression patterns for agronomic performance and trait efficacy.

The genetic elements contained in the ipd072Aa gene cassette of T-DNA Region of the selected event construct, Plasmid PHP74638, are described in Table 1.

TABLE 1
Description of Genetic Elements in the T-DNA Region of Plasmid PHP74638

| | Location on | | | |
|---|---|---|---|---|
| | T-DNA (Base | | Size | |
| | Pair Position) | Genetic Element | (bp) | Description |
| ipd072Aa | 20,903-20,939 | Intervening Sequence | 37 | DNA sequence used for cloning |
| gene | 20,940-21,353 | BSV(AY) Promoter | 414 | Promoter region from the banana |
| cassette | | | | streak virus (acuminata Yunnan |
| | | | | strain) genome (U.S. Pat. No. |
| | | | | 8,338,662) |
| | 21,354-21,372 | Intervening Sequence | 19 | DNA sequence used for cloning |
| | 21,373-22,228 | zm-HPLV9 Intron | 856 | Intron region from the Zea mays |
| | | | | ortholog of an Oryza sativa (rice) |
| | | | | hypothetical protein (zm-HPLV9) |
| | | | | gene, a predicted Zea mays |
| | | | | calmodulin 5 gene (WO Patent |
| | | | | 2016109157 [Abbitt and Shen, |
| | | | | 2016]) |
| | 22,229-22,237 | Intervening Sequence | 9 | DNA sequence used for cloning |
| | 22,238-22,498 | ipd072Aa | 261 | Insecticidal protein gene from |
| | | | | Pseudomonas chlororaphis |
| | | | | (Schellenberger et al., 2016) |
| | 22,499-22,504 | Intervening Sequence | 6 | DNA sequence used for cloning |
| | 22,505-23,077 | at-T9 Terminator | 573 | Terminator region from an |
| | | | | Arabidopsis thaliana putative gene |
| | | | | of the mannosebinding protein |
| | | | | superfamily (U.S. Pat. No. |
| | | | | 10,059,953) |
| | 23,078-23,116 | Intervening Sequence | 39 | DNA sequence used for cloning |

### Example 2. Transformation of Maize by Agrobacterium Transformation and Regeneration of Transgenic Plants Containing the ipd072Aa, Mo-Pat, and Pmi Genes

Maize event DP-051291-2 was created using three sequential transformation steps to (1) insert the specific integration site sequence in the maize genome to create the initial SSI “landing pad”; (2) reset the initial landing pad by replacing the first selectable marker with a second selectable marker to create the final SSI “landing pad”; and 3) insert the intended trait genes into the final SSI landing pad using site-specific integration (SSI). After each transformation, the maize genome was characterized using Southern-by-Sequencing (SbS) to ensure the intended insertion was present with sequence integrity and there were no unintended plasmid-derived sequences present in the genome.

DP-051291-2 maize event was produced by Agrobacterium-mediated SSI transformation with plasmid PHP74638. Agrobacterium-mediated SSI was essentially performed as described in U.S. patent application publication number 2017/0240911, herein incorporated by reference.

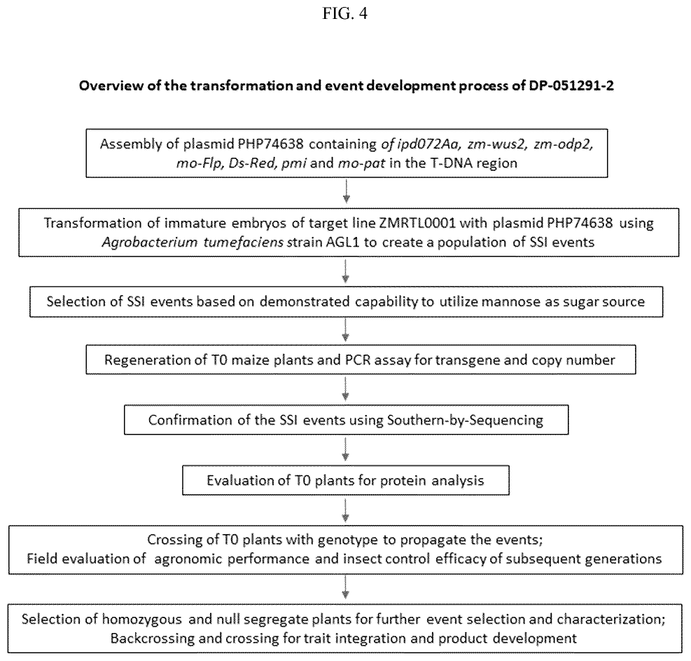

A total of 1289 immature embryos were infected with PHP74638. After the 105-day selection and regeneration process, a total of 120 TO plantlets were regenerated. Samples were taken from all TO plantlets for PCR analysis to verify the presence and copy number of the inserted ipd072Aa, pmi, and mo-pat genes. In addition to this analysis, the TO plantlets were analyzed by PCR for the presence of certain Agrobacterium binary vector backbone sequences, the developmental genes, zm-odp2 and zm-wus2 disclosed in U.S. Pat. Nos. 7,579,529 and 7,256,322, herein incorporated by reference in their entireties. Plants that were determined to contain a single copy of the inserted genes ipd072Aa, pmi, and mo-pat, no Agrobacterium backbone sequences, and no developmental genes were selected for further greenhouse propagation. Samples from those PCR selected TO quality events were collected for further analysis using Southern-by-Sequencing to confirm that the inserted genes were in the correct target locus (also referred to herein as the “landing pad”) without any gene disruptions. Maize events DP-051291-2 were confirmed to contain a single copy of the T-DNA (See Examples 3 and 4). These selected TO plants were assayed for trait efficacy and protein expression. TO plants meeting all criteria were advanced and crossed to inbred lines to produce seed for further testing. A schematic overview of the transformation and event development process is presented in FIG. 4.

### Example 3. Identification of Maize Events DP-051291-2

Polymerase chain reaction (PCR) amplification of unique regions within the introduced genetic elements can distinguish the test plants from their non-genetically modified counterparts and can be used to screen for the presence of the inserted T-DNA region of plasmid PHP74638.

For detection of the ipd072Aa, mo-pat, and pmi genes contained within DP-051291-2 maize as well as the genomic junction spanning the insertion site for DP-051291-2 maize, regions spanning between 72-bp and 113-bp were amplified using primers and probes specific for each unique sequence. Additionally, a 79-bp region of an endogenous reference gene, high mobility group A (HMG, GenBank accession number AF171874.1), was validated to be used in duplex with each assay for both qualitative and quantitative assessment of each assay and to demonstrate the presence of sufficient quality and quantity of DNA within the PCR reaction (Krech et al., 1999). Data from HMG was used in calculations regarding scoring. Data were compared to the performance of either the validated positive or copy number calibrator as well as negative genomic controls.

The real-time PCR reaction exploited the 5′ nuclease activity of the heat-activated DNA polymerase. Two primers and one probe annealed to the target DNA with the probe, which contained a 5′ fluorescent reporter dye and a 3′ quencher dye. With each PCR cycle, the reporter dye was cleaved from the annealed probe by the polymerase, emitting a fluorescent signal that intensified with each subsequent cycle. The cycle at which the emission intensity of the sample amplicon rose above the detection threshold was referred to as the C_{T} value. When no amplification occurred, there was no C_{T} calculated by the instrument and was assigned a C_{T} value of 40.

If copy number of the test samples was to be determined, copy number calibrators (samples known to contain defined copies of the gene of interest, e.g. 1 or 2 copies) were used as controls for both the endogenous gene and gene of interest. Fold differences were used to apply a copy number for each test sample. Fold difference, or fold change, is calculated using the formula of 2^{-ΔC}_{T}. The ΔC_{T} was calculated for the test samples and copy number calibrators as described above. A copy number of 1 was applied to the sample population producing a fold change between 0 and 0.7 with a maximum range of 0.75 when compared to the 2-copy calibrators. Likewise, a copy number of 2 was applied to a sample population producing a fold change ranging between 1.5 and 2.2 with a maximum range of 0.91 when compared to the single copy calibrators; and a copy number of 3 was applied to a sample population producing a fold change ranging between 1.3 and 1.5 with a maximum range of 0.35 when compared to the 2-copy calibrators.

Genomic DNA was isolated from DP-051291-2 maize leaf tissue for approximately 100 plants from each of the T1 and T2 generations. The DNA samples were extracted using an alkaline buffer comprised of sodium hydroxide, ethylenediaminetetraacetic acid disodium salt dihydrate (Na2-EDTA) and Tris hydrochloride. Approximately 5 ng of template DNA was used per reaction.

Each assay supporting the target event and transgenes were multiplexed with the HMG endogenous reference assay. Reaction mixes were prepared, each comprised of all components to support both the gene of interest and the endogenous gene for the PCR reaction. The base master mix, Bioline SensiFast™ Probe Lo-ROX master mix with 30% Bovine Serum Albumin (BSA) included as an additive was used. Individual concentrations of primer and probe varied per reaction between 600 nM and 900 nM for the primer and between 80 nM and 120 nM for the probe, dependent on the optimal concentration established during analysis validation. Assay controls included no template controls (NTC) which consisted of water or Tris-EDTA (TE) buffer (10 mM Tris pH 8.0, 1 mM EDTA) as well as copy number calibrator and negative controls, all of which were validated for each assay performed. The primer and probes used for each PCR analysis are provided in Tables 2 and 3. Annealing temperatures and number of cycles used during the PCR analyses are provided in Table 4.

Genomic DNA samples isolated from collected leaf samples of 200 DP-051291-2 maize plants (100 plants from each of the T1 and T2 generations), along with copy number calibrators, negative and no template controls (NTC), were subjected to quantitative real-time PCR (qPCR) amplification using primer pair and probes specific for genes ipd072Aa, mo-pat, and pmi, and the event-specific junction to the maize genome for the unique identification of the PHP74638-derived DNA insertion in DP-051291-2 maize. For assay and DNA quality monitoring, maize HMG was included in duplex with each reaction as an endogenous control. Each qPCR reaction was set up in a total volume of 6 μL with approximately 5-ng (1.0 μL of volume) of the isolated genomic DNA.

The PCR target sites and size of expected PCR products for each primer/probe set are shown in Table 2. Primer and probe sequence information supporting each targeted region are shown in Table 3. PCR reagents and reaction conditions are shown in Table 4. In this study approximately 5-ng of maize genomic DNA was used for all PCR reactions.

TABLE 2
PCR Genomic DNA Target Site and Expected Size of PCR Products

| | | Expected Size | Amplicon |
|---|---|---|---|
| Primer and | Targeted | of PCR Product | SEQ |
| Probe Set | Regions | (bp) | ID NO: |
| SEQ ID NOs: 6-8 | DP-051291-2 | 80 | 21 |
| | insertion | | |
| SEQ ID NOs: 9-11 | ipd072Aa | 72 | 22 |
| SEQ ID NOs: 12-14 | pmi | 113 | 23 |
| SEQ ID NOs: 15-17 | mo-pat | 76 | 24 |
| SEQ ID NOs: 18-20 | hmg-A | 79 | 25 |

TABLE 3
Primers and Probe Sequence and Amplicon for PCR Genomic DNA
Targeted Regions

| | | Length |
|---|---|---|
| Reagent | Sequence (5′ to 3′) | (base) |
| SEQ ID NO: 6 | CGCAATGTGTTATTAAGTTGTCTAAGC | 27 |
| forward primer | | |
| SEQ ID NO: 7 | CACGGTTAGCTGTACCAAATGC | 22 |
| reverse primer | | |
| SEQ ID NO: 8 | 6-FAM-TTTGTTTACACCACATACACG-MGB | 21 |
| probe | | |
| SEQ ID NO: 9 | ACAACAACGCCGTGAAGGA | 19 |
| forward primer | | |
| SEQ ID NO: 10 | CCAGATTGGTTTCACATACGTATCA | 25 |
| reverse primer | | |
| SEQ ID NO: 11 | 6-FAM-AGGGTCGGCTGATC-MGB | 14 |
| probe | | |
| SEQ ID NO: 12 | TGACTGTCAAAGGCCACGG | 19 |
| forward primer | | |
| SEQ ID NO: 13 | AGATGGACAAGTCTAGGTTCCACC | 24 |
| reverse primer | | |
| SEQ ID NO: 14 | 6-FAM- | 28 |
| probe | CCGTTTAGCGCGTGTTTACAACAAGCTG-BHQ | |
| SEQ ID NO: 15 | CATCGTGAACCACTACATCGAGAC | 24 |
| forward primer | | |
| SEQ ID NO: 16 | GTCGATCCACTCCTGCGG | 18 |
| reverse primer | | |
| SEQ ID NO: 17 | 6-FAM-ACCGTGAACTTCCGCACCGAGC-BHQ1 | 22 |
| probe | | |
| SEQ ID NO: 18 | TTGGACTAGAAATCTCGTGCTGA | 23 |
| forward primer | | |
| SEQ ID NO: 19 | GCTACATAGGGAGCCTTGTCCT | 22 |
| reverse primer | | |
| SEQ ID NO: 20 | VIC-GCGTTTGTGTGGATTG-MGB/NFQ | 16 |
| probe | | |

SEQ ID NO: 21: DP-051291-2 assay amplicon sequence
(80-bp; primer and probe binding sites are in bold and underlined)
CGCAATGTGTTATTAAGTTGTCTAAGCGTCAATTTGTTTACACCACATACA
CGTACACGCATTTGGTACAGCTAACCGTG
SEQ ID NO: 22: ipd072Aa assay amplicon sequence
(72-bp; primer and probe binding sites are in bold and underlined)
ACAACAACGCCGTGAAGGACCAGGGTCGGCTGATCGAGCCGCTCTCGTGA
TACGTATGTGAAACCAATCTGG
SEQ ID NO: 23: pmi assay amplicon sequence
(113-bp; primer and probe binding sites are in bold and underlined
TGACTGTCAAAGGCCACGGCCGTTTAGCGCGTGTTTACAACAAGCTGTAA
GAGCTTACTGAAAAAATTAACATCTCTTGCTAAGCTGGGGGTGGAACCTAGA
CTTGTCCATCT
SEQ ID NO: 24: mo-pat assay amplicon sequence
(76-bp; primer and probe binding sites are in bold and underlined
CATCGTGAACCACTACATCGAGACCTCCACCGTGAACTTCCGCACCGAGC
CGCAGACCCCGCAGGAGTGGATCGAC
SEQ ID NO: 25: hmg-A assay amplicon sequence
(79-bp; primer and probe binding sites are in bold and underlined)
TTGGACTAGAAATCTCGTGCTGATTAATTGTTTTACGCGTGCGTTTGTGTGG
ATTGTAGGACAAGGCTCCCTATGTAGC

TABLE 4
PCR Reagents and Reaction Conditions

| | | | Temperature | Time | |
|---|---|---|---|---|---|
| Step | Description | | (° C.) | (seconds) | Cycles |
| 1 | Initial Denaturation | | 95 | 300 | 1 |
| 2a | Amplification | Denaturation | 95 | 3 | 45^{a} |
| 2b | | Anneal/Extend | 60 | 30 | |

^{a}Thermal cycling was completed using a Roche LightCycler ® 480, 45 cycles for steps 2a and 2b were performed to obtain raw data to 40 cycles.

The results of the qPCR copy number analyses indicate stable integration and segregation of a single copy of the intended genes within the T-DNA of plasmid PHP74638, with demonstrated transfer to subsequent generations.

PCR products ranging in size between 72-bp to 113-bp, representing the insertion sites for DP-051291-2 maize as well as the genes within the T-DNA from plasmid PHP74638, were amplified and observed in leaf samples of DP-051291-2 maize as well as eight copy number calibrator genomic controls, but were absent in each of the eight negative genomic controls and eight NTC controls. For all samples, each assay was performed in duplex, analyzing for the event insertion site of DP-051291-2 and all genes a total of four times with the same results observed. For all data generated, C_{T} values, ΔC_{T} values, and copy numbers (if applicable) were calculated.

Using the maize endogenous reference gene HMG, a PCR product of 79-bp was amplified and observed in leaf samples from DP-051291-2 maize as well as eight copy number calibrator and eight negative genomic controls. Amplification of the endogenous gene was not observed in the eight NTC controls tested with no generation of C_{T} values.

To assess the sensitivity of the construct-specific PCR assays, DP-051291-2 maize DNA was diluted in control maize genomic DNA, resulting in test samples containing various amounts of DP-051291-2 maize (5-ng, 1-ng, 500-pg, 250-pg, 100-pg, 50-pg, 20-pg, 10-pg and 5-pg) in a total of 5-ng maize DNA. These various amounts of DP-051291-2 maize DNA correspond to 100%, 20%, 10%, 5%, 2%, 1%, 0.4%, 0.2% and 0.1% of DP-051291-2 maize DNA in total maize genomic DNA, respectively. The various amounts of DP-051291-2 maize DNA were subjected to real-time PCR amplification for ipd072Aa, mo-pat and pmi genes and the insertion site. Based on these analyses, the limit of detection (LOD) in 5-ng of total DNA for DP-051291-2 maize was determined to be approximately 20-pg for ipd072Aa (0.4%), 10-pg for mo-pat (0.2%), 10-pg for pmi (0.2%) and 20-pg (0.4%) of the insertion site representing event DP-051291-2. The determined sensitivity of each assay described is sufficient for many screening applications. Each concentration was tested a total of five times. At the point where amplification of the target tested was not detected in each replicate, the preceding concentration was determined to be the limit of sensitivity.

Real-time PCR analyses of event DP-051291-2 utilizing event-specific and construct-specific primer/probe sets for event DP-051291-2 confirm the stable integration and segregation of a single copy of the T-DNA of plasmid PHP74638 of the event in leaf samples tested, as demonstrated by the quantified detection of the event insertion site DP-051291-2 and the ipd072Aa, pmi, and mo-pat genes in DP-051291-2 maize. These results were reproducible among all the replicate qPCR analyses conducted. The maize endogenous reference gene assay for detection of hmg-A amplified as expected in all the test samples and negative controls and was not detected in the NTC samples. The sensitivity of each assay under the conditions described ranges from 20-pg to 10-pg DNA (0.4% to 0.2%), all sufficient for many screening applications by PCR.

### Example 4. Southern-by-Sequencing (SbS) Analysis of DP-051291-2 Maize for Integrity and Copy Number

Southern-by-Sequencing (SbS) analysis utilizes probe-based sequence capture, Next Generation Sequencing (NGS) techniques, and bioinformatics procedures to capture, sequence, and identify inserted DNA within the maize genome (Zastrow-Hayes et al., 2015). By compiling a large number of unique sequencing reads and mapping them against the intended insertion sequence (comprising the intended expression cassettes from PHP74638 and the landing pad sequences from PHP50742, including the Right Border and Left Border elements), linearized transformation plasmid maps, and the endogenous genomic reference, unique junctions due to inserted DNA are identified in the bioinformatics analysis. This information is used to determine the number and organization of insertions within the plant genome and confirm the absence of plasmid backbone or other unintended plasmid sequences.

Genomic DNA samples isolated from ten individual plants of the T1 generation of DP-051291-2 maize (five transgenic plants and five null segregant plants) were analyzed by SbS to determine the insertion copy number and organization within the plant genome, and to confirm the absence of plasmid backbone or other unintended sequences. Sbs was also performed on samples of control maize DNA from a maize plant that is not genetically modified and has the same genetic background as DP-051291-2 maize, but does not contain the DP-051291-2 insert, and positive control samples (control maize DNA spiked with PHP74638, PHP50742, PHP16072, PHP5096, PHP46438, PHP21139, or PHP31729 plasmid DNA) to confirm that the assay could reliably detect plasmid fragments within the genomic DNA.

Biotinylated capture probes for hybridization to plasmid sequences were designed and synthesized by Roche NimbleGen, Inc. (Madison, WI). The probe set was designed to target all sequences within the PHP74638, PHP50742, PHP16072, PHP5096, PHP46438, PHP21139, and PHP31729 plasmids.

Next-generation sequencing libraries were constructed for the DNA samples from DP-051291-2 maize plants, the control maize plant, and the positive control samples. SbS was performed as described by Zastrow-Hayes, et al. Plant Genome (2015). The sequencing libraries were hybridized to the capture probes through two rounds of hybridization to enrich the targeted sequences. Following NGS (Illumina, NextSeq), the sequencing reads were assessed for trimming and quality assurance. Reads were aligned against the maize genome and the plasmid sequences and reads that contain both genomic and plasmid sequence were identified as junction reads. Alignment of the junction reads to the transformation construct shows borders of the inserted DNA relative to the expected insertion.

To identify junctions that included endogenous maize sequences, the control maize genomic DNA library was captured and sequenced in the same manner as the DP-051291-2 maize plants. This increased the probability that the endogenous junctions captured by the probes would be detected in the control sample, so that they could be identified and removed in the DP-051291-2 maize samples.

SbS analysis of the five transgenic plants of the segregating T1 generation of DP-051291-2 maize that contained the inserted DNA yielded sequencing reads that were aligned to the intended insertion. Each of these plants contained two unique genome-insertion junctions, one at each end of the insertion; the two unique junctions were identical across the five plants. Each of the five null segregant plants from the T1 generation of DP-051291-2 maize that was negative for the DP-051291-2 insertion yielded sequencing reads for the endogenous genetic elements derived from the maize genome. There were no junctions between plasmid sequences and the maize genome detected in these plants, indicating that these plants did not contain any insertions derived from PHP74638, PHP50742, PHP16072, PHP5096, PHP46438, PHP21139, or PHP31729.

SbS analysis was conducted on the T1 generation of DP-051291-2 maize to determine the insertion copy number and organization within the plant genome, and to confirm the absence of plasmid backbone or other unintended sequences. Genomic DNA extracted from leaf tissue of ten individual plants from the segregating T1 generation of DP-051291-2 maize and one non-GM control maize plant, along with plasmid positive control samples, was analyzed by SbS using capture probes that cover the entire sequence of all plasmids utilized to create DP51291 maize: PHP74638, PHP50742, PHP16072, PHP5096, PHP46438, PHP21139, and PHP31729. The captured sequencing reads were aligned to the intended insertion and the sequences from PHP74638, PHP50742, PHP16072, PHP5096, PHP46438, PHP21139, and PHP31729. SbS detected a single copy of the inserted DNA, derived from PHP74638 and PHP50742, in five positive plants out of the ten plants from the segregating T1 generation of DP-051291-2 maize, and no insertions were detected in the five null segregant plants. Furthermore, no plasmid backbone or other unintended plasmid sequences were detected in the plants from the T1 generation of DP-051291-2 maize. No junctions between plasmid and maize genome sequences were detected in the non-GM control maize, indicating that it did not contain any insertions. SbS performed on the positive control samples (the control maize DNA spiked with PHP74638, PHP50742, PHP16072, PHP5096, PHP46438, PHP21139, or PHP31729 plasmid DNA) yielded sequence coverage across the entire length of each plasmid, indicating that the assay could reliably detect plasmid fragments within the maize genome.

SbS analysis of the T1 generation of DP-051291-2 maize demonstrated that DP-051291-2 maize contains a single copy of the inserted DNA derived from PHP74638 and PHP50742, with the expected organization, and that no additional insertions, plasmid backbone, or other unintended sequences are present in its genome.

### Example 5. Insect Efficacy of Maize Events DP-051291-2

Hybrid maize plants containing event DP-051291-2 which expresses the insect-active IPD072Aa protein for protection against certain coleopteran pests including corn rootworms (CRW) were evaluated in the field. The control consisted of maize plants in the same hybrid background (referred to as control maize), which did not contain event DP-051291-2 or other events active against CRW. Data were statistically analyzed using a linear mixed model.

Field testing was conducted in 13 locations located in commercial maize-growing regions of North America: Brookings, SD; Champaign, IL; Johnston, IA #1; Johnston, IA #2; Mankato, MN; Lanesboro, MN; Dunkerton, IA; Readlyn, IA; Shabonna, IL; Mineral Point, WI; Windfall, IN; York, NE; and Fairmont, NE. No efficacy data were collected at four of the 13 locations (sites Johnston, IA #1, Mankato, MN, Lanesboro, MN, and Windfall, IN) due to a low corn rootworm node-injury score (CRWNIS) below 0.75 on negative control roots.

Single-row plots (10 feet in length) were planted in a randomized complete block experimental design with three replications. Each replication included DP-051291-2 maize as well as negative control maize. Prior to planting, 200 kernels from each seed lot were characterized by PCR analysis to confirm the presence of the DP-051291-2 event. At all locations, the trial was planted in fields with a history of natural corn rootworm infestations. Additionally, at all 13 sites an eight-foot section of each plot was manually infested when plants reached the V2-V4 growth stage with non-diapausing western corn rootworm eggs. Eggs were infested at a targeted infestation rate of approximately 750 eggs/plant. Eggs were injected into the soil approximately 4 inches deep and approximately 2 inches on both sides of each plant.

Injury from larval feeding on roots was evaluated when plants were at approximately the R2 growth stage. A target of five maize plants from each plot were tagged, manually dug from the ground, washed clean of soil with pressurized water, and visually evaluated for the amount of larval feeding contained on each root. Only healthy, representative plants were selected for data collection. The corn rootworm node-injury score (CRWNIS) was recorded for each plant using the Iowa State 0-3 node-injury scale described in Oleson et al., 2005.

The mean node-injury root rating results from CRW for both DP-051291-2 maize and control maize are provided in Table 5. These results indicate that the insect-active IPD072Aa protein expressed in DP-051291-2 maize provides protection from feeding against CRW compared to the negative control maize.

TABLE 5
Across-Site Efficacy Results Against Corn Rootworm

| | Number | Number | Mean | Standard | 95% |
|---|---|---|---|---|---|
| | of | of | Node-Injury | Error of | Confidence |
| Maize Line | Plots | Plants | Rating^{a} | Mean | Interval |
| DP-051291-2 | 27 | 130 | 0.17 a | 0.06 | (0.0524, 0.2876) |
| Control | 27 | 120 | 2.12 b | 0.06 | (2.0024, 2.2376) |

^{a}Mean node-injury ratings followed by different letters indicate a statistically significant difference; (P-value <0.05)

### Example 6. Agronomic and Yield Field Evaluations of Maize Events DP-051291-2

Agronomic field trials containing DP-051291-2 were to generate yield data and to evaluate other agronomic characteristics. All inbred and hybrid materials tested for an event were generated from a single TO plant.

### Hybrid Trials

Across two years of testing, seven hybrid trials were planted across a total of seventy growing locations, with two replicates of the entry list at each location. Grain was harvested from 52 of the 70 locations. Experiments were run as a randomized split-plot treatment design. Main plots were hybrid background and subplots were transgene status (Event DP-051291-2 or WT). Subplots were randomized within main plots, and main plots were randomized within each replication. Two replications were established in each field location in each year. Plots were 4 rows 76 cm apart and ranged from 4.4 to 5.3 m in length, with a 0.5 m alley between each plot. Various observations and data were collected at each planted location throughout the growing season. The following agronomic characteristics were analyzed for comparison to a wild type entry (WT). Data generated for the hybrid agronomic trials included the following agronomic traits (Table 6):
1.) Ear height (EARHT): Measurement from the ground to the attachment point of the highest developed ear on the plant. Ear height is measured in inches.
2.) Growing degree units to shed (GDUSHD): Measurement records the total accumulated growing degree units when 50% of the plants in the plot have tassels that are shedding pollen. A single day equivalent is approximately 1.5 growing degrees units for this data set.
3.) Growing degree units to silk (GDUSLK): Measurement records the total accumulated growing degree units when 50% of the plants in the plot have exposed silks. A single day equivalent is approximately 1.5 growing degrees units for this data set.
4.) Plant height (PLTHT): Measurement by drones from the ground to the base of the flag leaf. Plant height is measured in inches.
5.) Moisture (MST): Measurement of the percent grain moisture at harvest.
6.) Yield: Recorded weight of grain harvested from each plot. Calculations of reported bu/acre yields were made by adjusting to measured moisture of each plot.

### Inbred Trials

Inbred trials were planted across 13 locations with 2 replicates of the entry list at each location. Grain was harvested from 4 locations for analysis. Experiments were run as a randomized split-plot treatment design. Main plots were inbred background and subplots were transgene status (Event DP-051291-2 or WT). Agronomic data and observations were collected for the inbred trials and analyzed for comparison to a wild type entry (WT). Data generated for the inbred trials included the following agronomic traits (Table 7):
1.) Ear height (EARHT): Measurement from the ground to the attachment point of the highest developed ear on the plant. Ear height is measured in inches.
2.) Growing degree units to shed (GDUSHD): Measurement records the total accumulated growing degree units when 50% of the plants in the plot have tassels that are shedding pollen. A single day equivalent is approximately 1.5 growing degrees units for this data set.
3.) Growing degree units to silk (GDUSLK): Measurement records the total accumulated growing degree units when 50% of the plants in the plot have exposed silks. A single day equivalent is approximately 1.5 growing degrees units for this data set
4.) Plant height (PLTHT): Measurement from the ground to the base of the flag leaf. Plant height is measured in inches.
5.) Photometry Kernels Per Ear (PHTKPE): Number of Kernels per Ear determined by ear photometry.

### Trial Results

To evaluate the hybrid data, a mixed model framework was used to perform multi location analysis. In the multi-location analysis, main effect event is considered as fixed effect. Factors for location, background, tester, tester by event, location by background, location by tester, location by event, location by tester by event and rep within location are considered as random effects. The spatial effects including range and plot within locations were considered as random effects to remove the extraneous spatial noise. The heterogeneous residual was assumed with autoregressive correlation as AR1*AR1 for each location. The T-tests were conducted to compare event with WT. A difference was considered statistically significant if the P-value of the difference was less than 0.05. Yield analysis was by ASREML (VSN International Ltd; Best Linear Unbiased Prediction; Cullis, B. R et al (1998) Biometrics 54: 1-18, Gilmour, A. R. et al (2009); ASReml User Guide 3.0, Gilmour, A. R., et al (1995) Biometrics 51: 1440-50).

To evaluate the inbred data, a mixed model framework was used to perform multi location analysis. In the multi-location analysis, main effect event is considered as fixed effect. Factors for location, background, event, location by event and rep within location are considered as random effects. The spatial effects including range and plot within locations were considered as random effects to remove the extraneous spatial noise. The heterogeneous residual was assumed with autoregressive correlation as AR1*AR1 for each location. The T-tests were conducted to compare event with WT. A difference was considered statistically significant if the P-value of the difference was less than 0.05. Yield analysis was by ASREML (VSN International Ltd; Best Linear Unbiased Prediction; Cullis, B. Ret at (1998) Biometrics 54: 1-18, Gilmour, A. R. et at (2009); ASReml User Guide 3.0, Gilmour, A. R., et at (1995) Biometrics 51: 1440-50).

TABLE 6
Hybrid performance of events DP-051291-2 compared to base entry

| | | Number of | | | | |
|---|---|---|---|---|---|---|
| | | plots with | Predicted | Standard | | |
| Trait (unit) | Event | data | value | Error | Diff | P-Value |
| EARHT (in.) | DP-051291-2 | 786 | 52.71 | 2.19 | 0.34 | 0.3370 |
| EARHT (in.) | WT (base comparator) | 880 | 52.37 | 2.23 | | |
| GDUSHD (GDU) | DP-051291-2 | 360 | 133.5 | 2 | 0.5 | 0.0209 |
| GDUSHD (GDU) | WT (base comparator) | 372 | 133 | 2 | | |
| GDUSLK (GDU) | DP-051291-2 | 316 | 136.1 | 1.5 | 0.3 | 0.2509 |
| GDUSLK (GDU) | WT (base comparator) | 324 | 135.8 | 1.5 | | |
| MST (%) | DP-051291-2 | 3778 | 19.4 | 0.3 | 0.1 | 0.0001 |
| MST (%) | WT (base comparator) | 4134 | 19.3 | 0.3 | | |
| PLTHT (in.) | DP-051291-2 | 2984 | 101.982 | 1.487 | 0.576 | 0.0000 |
| PLTHT (in.) | WT (base comparator) | 3272 | 101.406 | 1.487 | | |
| YIELD (bu/acre) | DP-051291-2 | 3747 | 193.8 | 3.7 | −0.4 | 0.3131 |
| YIELD (bu/acre) | WT (base comparator) | 4104 | 194.2 | 3.7 | | |

TABLE 7
Inbred performance of events DP-051291-2 compared to base entry

| | | Number of | | | | |
|---|---|---|---|---|---|---|
| | | plots with | Predicted | Standard | | |
| Trait (unit) | Event | data | value | Error | Diff | P-Value |
| EARHT (in.) | DP-051291-2 | 254 | 28.18 | 1.46 | −0.35 | 0.5438 |
| EARHT (in.) | WT (base comparator) | 336 | 28.53 | 1.46 | | |
| GDUSHD (GDU) | DP-051291-2 | 193 | 140.7 | 1.9 | 0.4 | 0.2868 |
| GDUSHD (GDU) | WT (base comparator) | 256 | 140.3 | 1.9 | | |
| GDUSLK (GDU) | DP-051291-2 | 192 | 141.8 | 2.3 | 0.7 | 0.0849 |
| GDUSLK (GDU) | WT (base comparator) | 255 | 141.1 | 2.3 | | |
| PHTKPE | DP-051291-2 | 215 | 367.1 | 13.7 | 13.5 | 0.0809 |
| PHTKPE | WT (base comparator) | 287 | 353.6 | 13.6 | | |
| PLTHT (in.) | DP-051291-2 | 212 | 65.604 | 2.939 | 0.652 | 0.3951 |
| PLTHT (in.) | WT (base comparator) | 282 | 64.952 | 2.938 | | |

### Example 7. Protein Expression and Concentration Protein Extraction

The concentrations of IPD072Aa, PAT, and PMI proteins were determined using quantitative enzyme-linked immunosorbent assay (ELISA) methods that have been internally validated to demonstrate method suitability. For analysis of IPD072Aa, PAT, and PMI protein concentrations, non-herbicide treated processed tissue subsamples were weighed at the following target weights: 5 mg for pollen, 10 mg for leaf, 20 mg for grain and root, and 30 mg for forage. Pollen, leaf, grain, and forage samples analyzed for IPD072Aa protein were extracted with 0.60 ml of chilled 25% StabilZyme Select in phosphate buffered saline containing polysorbate 20 (PBST). Root samples analyzed for IPD072Aa protein were extracted in chilled H5 buffer, which was comprised of 90 mM HEPES, 140 mM sodium chloride, 1.0% polyethylene glycol, 1.0% PVP40, 1.0% bovine serum albumin, 0.007% thimerosal, and 0.3% polysorbate 20. Samples analyzed for PAT and PMI proteins were extracted with 0.60 ml of chilled PBST. All extracted samples were centrifuged, and then supernatants were removed and prepared for analysis.

### Determination of IPD072Aa Protein Concentration

Prior to analysis, samples were diluted as applicable with 25% StabilZyme Select in PBST. Standards (typically analyzed in triplicate wells) and diluted samples (typically analyzed in duplicate wells) were incubated in a plate pre-coated with an IPD072Aa-specific antibody. Following incubation, unbound substances were washed from the plate and the bound IPD072Aa protein was incubated with a different IPD072Aa-specific antibody conjugated to the enzyme horseradish peroxidase (HRP). Unbound substances were washed from the plate. Detection of the bound IPD072Aa-antibody complex was accomplished by the addition of substrate, which generated a colored product in the presence of HRP. The reaction was stopped with an acid solution and the optical density (OD) of each well was determined using a plate reader.

### Determination of PAT Protein Concentration

Prior to analysis, samples were diluted as applicable in PBST. Standards (typically analyzed in triplicate wells) and diluted samples (typically analyzed in duplicate wells) were co-incubated with a PAT-specific antibody conjugated to the enzyme HRP in a plate pre-coated with a different PAT-specific antibody. Following incubation, unbound substances were washed from the plate. Detection of the bound PAT-antibody complex was accomplished by the addition of substrate, which generated a colored product in the presence of HRP. The reaction was stopped with an acid solution and the OD of each well was determined using a plate reader.

### Determination of PMI Protein Concentration

Prior to analysis, samples were diluted as applicable in PBST. Standards (typically analyzed in triplicate wells) and diluted samples (typically analyzed in duplicate wells) were incubated in a plate pre-coated with a PMI-specific antibody. Following incubation, unbound substances were washed from the plate and the bound PMI protein was incubated with a different PMI-specific antibody conjugated to the enzyme HRP. Unbound substances were washed from the plate. Detection of the bound PMI-antibody complex was accomplished by the addition of substrate, which generated a colored product in the presence of HRP. The reaction was stopped with an acid solution and the OD of each well was determined using a plate reader.

### Calculations for Determining Protein Concentrations

SoftMax Pro GxP (Molecular Devices) microplate data software was used to perform the calculations required to convert the OD values obtained for each set of sample wells to a protein concentration value.

A standard curve was included on each ELISA plate. The equation for the standard curve was derived by the software, which used a quadratic fit to relate the OD values obtained for each set of standard wells to the respective standard concentration (ng/ml). The sample concentration values were adjusted for a dilution factor expressed as 1:N by multiplying the interpolated concentration by N.

$$Adjusted Concentration = Interpolated Sample Concentration \times Dilution Factor$$

Adjusted sample concentration values obtained from SoftMax Pro GxP software were converted from ng/ml to ng/mg sample weight as follows:

$$Sample Concentration ( ng protein / mg sample weight ) = Sample Concentration ( ng / ml ) \times \frac{Extraction Buffer Volume ( ml )}{Sample Target Weight ( mg )}$$

The reportable assay lower limit of quantification (LLOQ) in ng/ml was calculated as follows:

$$Reportable Assay LLOQ ( ng / ml ) = ( lowest standard concentration - 10 \% ) \times minimum dilution$$

The LLOQ, in ng/mg sample weight, was calculated as follows:

$$LLOQ = Reportable Assay LLOQ ( ng / ml ) \times \frac{Extraction Buffer Volume ( ml )}{Sample Target Weight ( mg )}$$

### Results

Protein concentration results (means, standard deviations, and ranges) were determined for IPD072Aa, PAT, and PMI proteins in root (V6, V9, R1, and R4 growth stages), leaf (V9, R1 and R4 growth stages), pollen (R1 growth stage), forage (R4 growth stage), and grain (R6 growth stage) from DP-051291-2 maize as shown in Table 8.

TABLE 8
Expressed Trait Protein Concentration Results from DP-051291-2 maize

| | | | | | Number of |
|---|---|---|---|---|---|
| | | | | | Samples |
| | | | | | <LLOQ/ |
| | | Protein Concentration | | | Total |
| | Tissue | (ng/mg Tissue Dry Weight) | | | Number |
| | (Growth | | | Sample | of Samples |
| Protein | Stage) | Mean ± SD | Range | LLOQ^{a} | Reported |
| IPD072Aa | Root (V6) | 76 ± 31 | 17-140 | 0.11 | 0/24 |
| | Root (V9) | 140 ± 51 | 63-230 | 0.11 | 0/24 |
| | Root (R1) | 180 ± 85 | 66-330 | 0.11 | 0/24 |
| | Root (R4) | 140 ± 80 | 36-280 | 0.11 | 0/23^{b} |
| | Leaf (V9) | 69 ± 33 | 23-140 | 0.054 | 0/24 |
| | Leaf (R1) | 68 ± 25 | 31-110 | 0.054 | 0/24 |
| | Leaf (R4) | 53 ± 28 | 18-120 | 0.054 | 0/24 |
| | Pollen (R1) | 1.2 ± 1.4 | 0.25-7.1 | 0.11 | 0/24 |
| | Forage (R4) | 34 ± 20 | 9.2-88 | 0.018 | 0/24 |
| | Grain (R6) | 4.1 ± 3.6 | 0.051-12 | 0.027 | 0/24 |
| PAT | Root (V6) | 34 ± 8.0 | 16-51 | 0.054 | 0/24 |
| | Root (V9) | 26 ± 6.2 | 14-36 | 0.054 | 0/24 |
| | Root (R1) | 21 ± 4.6 | 15-33 | 0.054 | 0/24 |
| | Root (R4) | 10 ± 2.3 | 6.6-17 | 0.054 | 0/24 |
| | Leaf (V9) | 38 ± 5.9 | 30-49 | 0.11 | 0/24 |
| | Leaf (R1) | 40 ± 4.5 | 32-50 | 0.11 | 0/24 |
| | Leaf (R4) | 21 ± 3.2 | 15-28 | 0.11 | 0/24 |
| | Pollen (R1) | 67 ± 7.5 | 58-83 | 0.22 | 0/24 |
| | Forage (R4) | 15 ± 2.7 | 11-22 | 0.036 | 0/24 |
| | Grain (R6) | 5.7 ± 1.8 | 2.3-9.0 | 0.054 | 0/24 |
| PMI | Root (V6) | 8.3 ± 2.7 | 2.7-13 | 0.27 | 0/24 |
| | Root (V9) | 6.9 ± 2.3 | 2.9-12 | 0.27 | 0/24 |
| | Root (R1) | 4.8 ± 1.6 | 2.3-8.4 | 0.27 | 0/24 |
| | Root (R4) | 3.7 ± 1.0 | 2.4-6.3 | 0.27 | 0/24 |
| | Leaf (V9) | 8.9 ± 2.7 | 4.4-14 | 0.54 | 0/24 |
| | Leaf (R1) | 13 ± 4.6 | 7.2-26 | 0.54 | 0/24 |
| | Leaf (R4) | 29 ± 6.0 | 17-43 | 0.54 | 0/24 |
| | Pollen (R1) | 29 ± 4.4 | 19-37 | 1.1 | 0/24 |
| | Forage (R4) | 9.2 ± 1.3 | 6.8-12 | 1.8 | 0/24 |
| | Grain (R6) | 4.1 ± 1.7 | 1.7-9.3 | 0.27 | 0/24 |

Note:
Growth stages (Abendroth et al., 2011).
^{a}Lower limit of quantification (LLOQ) in ng/mg tissue dry weight.
^{b}One root sample was not analyzed for IPD072Aa protein due to insufficient quantity. This sample was analyzed for PAT and PMI protein concentrations.

The above description of various illustrated embodiments of the disclosure is not intended to be exhaustive or to limit the scope to the precise form disclosed. While specific embodiments of and examples are described herein for illustrative purposes, various equivalent modifications are possible within the scope of the disclosure, as those skilled in the relevant art will recognize. The teachings provided herein can be applied to other purposes, other than the examples described above. Numerous modifications and variations are possible in light of the above teachings and, therefore, are within the scope of the appended claims.

These and other changes may be made in light of the above detailed description. In general, in the following claims, the terms used should not be construed to limit the scope to the specific embodiments disclosed in the specification and the claims.

The entire disclosure of each document cited (including patents, patent applications, journal articles, abstracts, manuals, books or other disclosures) in the Background, Detailed Description, and Examples is herein incorporated by reference in their entireties.

Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight; temperature is in degrees celsius; and pressure is at or near atmospheric.

1. A corn plant comprising the genotype of the corn event DP-051291-2, wherein said genotype comprises a nucleotide sequence as set forth in SEQ ID NO: 26 and SEQ ID NO: 29.

2. The corn plant of claim 1, wherein said genotype comprises the nucleotide sequence set forth in SEQ ID NO: 27 and SEQ ID NO: 30.

3. The corn plant of claim 1, wherein said genotype comprises the nucleotide sequence set forth in SEQ ID NO: 28 and SEQ ID NO: 31.

4. (canceled)

5. (canceled)

6. (canceled)

7. (canceled)

8. (canceled)

9. (canceled)

10. Seed comprising corn event DP-051291-2, wherein said seed comprises a DNA molecule chosen from SEQ ID NO: 26 and SEQ ID NO: 29, wherein a representative sample of the corn event DP-051291-2 seed of has been deposited with American Type Culture Collection (ATCC) with Accession No. PTA-127358.

11. A corn plant, or part thereof, grown from the seed of claim 10.

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. A method of detecting the presence of a nucleic acid molecule that is unique to event DP-051291-2 in a sample comprising corn nucleic acids, the method comprising:
a. contacting the sample with a pair of primers that, when used in a nucleic-acid amplification reaction with genomic DNA from event DP-051291-2 produces an amplicon that is diagnostic for event DP-051291-2;
b. performing a nucleic acid amplification reaction, thereby producing the amplicon that is diagnostic for event DP-051291-2; and
c. detecting the amplicon that is diagnostic for event DP-051291-2.

38. The method of claim 37, wherein the nucleic acid molecule that is diagnostic for event DP-051291-2 is an amplicon produced by the nucleic acid amplification chain reaction.

39. The method of claim 37, wherein the method further comprises contacting the sample with a probe.

40. The method of claim 39, wherein the probe comprises a detectable label.

41. The method of claim 40, wherein the detectable label is covalently associated with the probe.

42. (canceled)

43. (canceled)

44. (canceled)

45. (canceled)

46. (canceled)

47. (canceled)

48. (canceled)

49. (canceled)

50. (canceled)

51. A method of modifying the DP-051291-2 corn event, wherein a representative sample of seed of said corn event was deposited with the ATCC with accession number PTA-127358, comprising applying genome engineering technology to a DNA sequence of said DP-051291-2 corn event to modify the DNA of said corn event.

52. The method of claim 51, comprising modifying the DNA of said DP-051291-2 corn event to produce a modified DNA sequence having at least 90% sequence identity to SEQ ID NO: 3.

53. The method of claim 51, comprising modifying the DNA of said DP-051291-2 corn event to produce a modified DNA sequence having all or a portion of SEQ ID NO: 26 or SEQ ID NO: 29 duplicated in said modified DNA sequence.

54. The method of claim 51, comprising modifying the DNA of said DP-051291-2 corn event to produce a modified DNA sequence comprising an excision from SEQ ID NO: 3.

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. A method of controlling Coleopteran insects, comprising exposing the Coleopteran insects to insect resistant maize plants of event DP-051291-2.

67. The method of claim 66, wherein the Coleopteran insect is Corn Rootworm.

68. The method of claim 66, wherein damage from the Coleopteran insect is controlled for maize roots from event DP-051291-2.

69. A corn plant comprising the genotype of corn event DP-051291-2, wherein the plant comprises the nucleotide sequence set forth in SEQ ID NO: 3, or a nucleotide sequence having at least 98% sequence identity to SEQ ID NO: 3.

70. A corn plant comprising the genotype of corn event DP-051291-2, wherein said genotype comprises the nucleotide sequence set forth in one of SEQ ID NOs: 26-28 and SEQ ID NOs: 29-31, or a nucleotide sequence having 1 nucleotide change in one of SEQ ID NOs: 26-28 and SEQ ID NOs: 29-31.


SEQUENCE LISTING (31 sequences)

SEQ ID NO: 1 - DNA, 66641 bp, synthetic construct
  source 1..66641: mol_type = other DNA
gtttacccgccaatatatcctgtcaaacactgatagtttaaactgaaggcgggaaacgacaatctgatcatgagcctagttagttaggggcagctaacgtcccctagctaactagttgctattaacctttagcctagtttagtgcggtcgcgctagtgaactagtctaatgggtgcctagcttagattagatccccgttcggcttagactaggttagtcggatagggctagtctagtggcttagcttggaactgttaagctaggctgttagtgcgggacttctaggctaacctattgacccctaacgcctagaacctaagaaacgctaaggacacctagctaactttggcgacgtcccgaggtccgattccggctaactaactagcttaagggttaccggccggccacactgatagtttaaactgaaggcgggaaacgacaatctgatcatgagcggagaattaagggagtcacgttatgacccccgccgatgacgcgggacaagccgttttacgtttggaactgacagaaccgcaacgattgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcccggcggatctcgactctagaggatcgctcaggaaggccgctgagatagagccatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgctatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccaacagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgagagcaacgcgagctgccactgctcttcactgatgtctctggaatggaaggaggaggaagtgagcatagcgttggtgcgttgctgtcaagggcgaattgtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgggtaccatttaaattgcgcccgccacggccgtggaggtcgtattccggtcagcttgcatccctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccatggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcttcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcgggcatggcggcgcgcagctcttcagtgtctggaacgacacttaagcgtacgtgccggcctggctctccgaaagggcgtattccagcacactggcggccgttactagacccaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggcctccggattcttatgtgcttctagtctccaaatgtggttgatagttattttgctctaagatcaacagtaatgaagtataaatcatcgttgtggtgtgctactcggttaattgagcattaacacacacaaacatgacgaggatggtataatctccaaaaatgtgtactttgttaggtgggaccctatagccttgattaatgtgctatgttaggcatgcctggaaacgtgtgacgcatatgttttgtgaacctgttgatattatatgtgcttttatattaccatattttattaaaatactaatatttattactagtaagatataacattctatctagcttaaaaactaaccataaatattccataataactagatttaccaaactaatatactaaatatacataataaatacaaaattaacaagacaataatcaatatttatgagcttaatatatttagacattatggttggtcgacgataatcatgctaacttttcgtaattgcttgattgaaatatgcttagaataatgcctctttgttctacatggcaaatagggaccattatggtgtaacaccctgggaaccacaaacaccccgaaatgctactaaactacacaactaaccttcatatataaaatttcgacagcatctcctttgaaaatttgcatagacgtggaagcaacagagtataaacagatatcatgataagaaaacatactagacattaataatctgctagaaatgggaagaatcacgcgtaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccaacaatgccccagttcgacatcctctgcaagaccccccccaaggtgctcgtgaggcagttcgtggagaggttcgagaggccctccggcgagaagatcgccctctgcgccgccgagctcacctacctctgctggatgatcacccacaacggcaccgccattaagagggccaccttcatgtcatacaacaccatcatctccaactccctctccttcgacatcgtgaacaagtccctccagttcaaatacaagacccagaaggccaccatcctcgaggcctccctcaagaagctcatccccgcctgggagttcaccatcatcccctactacggccagaagcaccagtccgacatcaccgacatcgtgtcatccctccagcttcagttcgagtcctccgaggaggctgacaagggcaactcccactccaagaagatgctgaaggccctcctctccgagggcgagtccatctgggagatcaccgagaagatcctcaactccttcgagtacacctccaggttcactaagaccaagaccctctaccagttcctcttcctcgccaccttcatcaactgcggcaggttctcagacatcaagaacgtggaccccaagtccttcaagctcgtgcagaacaagtacctaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtgtcatcatccagtgcctcgtgaccgagaccaagacctccgtgtccaggcacatctacttcttctccgctcgcggcaggatcgaccccctcgtgtacctcgacgagttcctcaggaactcagagcccgtgctcaagagggtgaacaggaccggcaactcctcctccaacaagcaggagtaccagctcctcaaggacaacctcgtgaggtcctacaacaaggccctcaagaagaacgccccctactccatcttcgccatcaagaacggccccaagtcccacatcggtaggcacctcatgacctccttcctctcaatgaagggcctcaccgagctcaccaacgtggtgggcaactggtccgacaagagggcctccgccgtggccaggaccacctacacccaccagatcaccgccatccccgaccactacttcgccctcgtgtcaaggtactacgcctacgaccccatctccaaggagatgatcgccctcaaggacgagactaaccccatcgaggagtggcagcacatcgagcagctcaagggctccgccgagggctccatcaggtaccccgcctggaacggcatcatctcccaggaggtgctcgactacctctcctcctacatcaacaggaggatctgagttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcggtcaccgaattcccatggagtcaaagattcaaatagaggacctaacagaactcgccgtaaagactggcgaacagttcatacagagtctcttacgactcaatgacaagaagaaaatcttcgtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactaagctgaccgaagctggccgctctagaactagtggatctcgatgtgtagtctacgagaagggttaaccgtctcttcgtgagaataaccgtggcctaaaaataagccgatgaggataaataaaatgtggtggtacagtacttcaagaggtttactcatcaagaggatgcttttccgatgagctctagtagtacatcggacctcacatacctccattgtggtgaaatattttgtgctcatttagtgatgggtaaattttgtttatgtcactctaggttttgacatttcagttttgccactcttaggttttgacaaataatttccattccgcggcaaaagcaaaacaattttattttacttttaccactcttagctttcacaatgtatcacaaatgccactctagaaattctgtttatgccacagaatgtgaaaaaaaacactcacttatttgaagccaaggtgttcatggcatggaaatgtgacataaagtaacgttcgtgtataagaaaaaattgtactcctcgtaacaagagacggaaacatcatgagacaatcgcgtttggaaggctttgcatcacctttggatgatgcgcatgaatggagtcgtctgcttgctagccttcgcctaccgcccactgagtccgggcggcaactaccatcggcgaacgacccagctgacctctaccgaccggacttgaatgcgctaccttcgtcagcgacgatggccgcgtacgctggcgacgtgcccccgcatgcatggcggcacatggcgagctcagaccgtgcgtggctggctacaaatacgtaccccgtgagtgccctagctagaaacttacacctgcaactgcgagagcgagcgtgtgagtgtagccgagtagatcccccgggctgcaggtcgactctagaggatccaccggtcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgacgaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagcggccgctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtggcgagctctcgagtcgatcgctatcaactttgtatagaaaagttgggccgaattcgagctcggtacggccagaatggcccggaccgggttaccgaattcgagctcggtaccctgggatcagcttcgctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtggcgagctgctagcgaagttcctattccgaagttcctattctctagaaagtataggaacttcagatccaccgggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggggtggaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcgaccttcttatgtgcttctagtctccaaatgtggttgatagttattttgctctaagatcaacagtaatgaagtataaatcatcgttgtggtgtgctactcggttaattgagcattaacacacacaaacatgacgaggatggtataatctccaaaaatgtgtactttgttaggtgggaccctatagccttgattaatgtgctatgttaggcatgcctggaaacgtgtgacgcatatgttttgtgaacctgttgatattatatgtgcttttatattaccatattttattaaaatactaatatttattactagtaagatataacattctatctagcttaaaaactaaccataaatattccataataactagatttaccaaactaatatactaaatatacataataaatacaaaattaacaagacaataatcaatatttatgagcttaatatatttagacattatggttggtcgacgataatcatgctaacttttcgtaattgcttgattgaaatatgcttagaataatgcctctttgttctacatggcaaatagggaccattatggtgtaacaccctgggaaccacaaacaccccgaaatgctactaaactacacaactaaccttcatatataaaatttcgacagcatctcctttgaaaatttgcatagacgtggaagcaacagagtataaacagatatcatgataagaaaacatactagacattaataatctgctagaaatgggaagaatcctaacttgacgactgcgtaactgactagagtcacacttagctgaccctagtcacttacaactgacttcgtgtcctaggcttaggctactgctagtccgcggtgtatccgtgatcgagttggcgccagacggaatctgttctccatcgctgacatcctcgagtagatcacattcaagcttgatatcgaattcctgcagcccatccctcagccgcctttcactatcttttttgcccgagtcattgtcatgtgaaccttggcatgtataatcggtgaattgcgtcgattttcctcttataggtgggccaatgaatccgtgtgatcgcgtctgattggctagagatatgtttcttccttgttggatgtattttcatacataatcatatgcatacaaatatttcattacactttatagaaatggtcagtaataaaccctatcactatgtctggtgtttcattttatttgcttttaaacgaaaattgacttcctgattcaatatttaaggatcgtcaacggtgtgcagttactaaattctggtttgtaggaactatagtaaactattcaagtcttcacttattgtgcactcacctctcgccacatcaccacagatgttattcacgtcttaaatttgaactacacatcatattgacacaatattttttttaaataagcgattaaaacctagcctctatgtcaacaatggtgtacataaccagcgaagtttagggagtaaaaaacatcgccttacacaaagttcgctttaaaaaataaagagtaaattttactttggaccacccttcaaccaatgtttcactttagaacgagtaattttattattgtcactttggaccaccctcaaatcttttttccatctacatccaatttatcatgtcaaagaaatggtctacatacagctaaggagatttatcgacgaatagtagctagcatactcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgttttttttttccgtcacggtctcgatctttggccttggtagtttgggtgggcgagaggcggcttcgtgcgcgcccagatcggtgcgcgggaggggcgggatctcgcggctggggctctcgccggcgtggatcaggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaaggatccacacgacaccatgtcccccgagcgccgccccgtcgagatccgcccggccaccgccgccgacatggccgccgtgtgcgacatcgtgaaccactacatcgagacctccaccgtgaacttccgcaccgagccgcagaccccgcaggagtggatcgacgacctggagcgcctccaggaccgctacccgtggctcgtggccgaggtggagggcgtggtggccggcatcgcctacgccggcccgtggaaggcccgcaacgcctacgactggaccgtggagtccaccgtgtacgtgtcccaccgccaccagcgcctcggcctcggctccaccctctacacccacctcctcaagagcatggaggcccagggcttcaagtccgtggtggccgtgatcggcctcccgaacgacccgtccgtgcgcctccacgaggccctcggctacaccgcccgcggcaccctgcgcgccgccggctacaagcacggcggctggcacgacgtcggcttctggcagcgcgacttcgagctgccggccccgccgcgcccggtgcgcccggtgacgcagatctgagtcgacctgcaggcatgccgctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtggcgagctaatgcggcccgaataacttcgtatagcatacattatacgaagttatacctggtggcgccgctaggggctgcaggaattcctgcagcccgggggatccactagttctagagcggccgacctcgacagatctaagcttactagtgccgtgggtcgtttaagctgccgctgtacctgtgtcgtctggtgccttctggtgtacctgggaggttgtcgtctatcaagtatctgtggttggtgtcatgagtcagtgagtcccaatactgttcgtgtcctgtgtgcattatacccaaaactgttatgggcaaatcatgaataagcttgatgttcgaacttaaaagtctctgctcaatatggtattatggttgtttttgttcgtctcctaatatttgcctgggatcaaattttattggctggtgttcatttgacctccatgttcttgctaggctccattttttactctacagccataatatgtttgattgtttggtttgttctttgttgtacacctggttctgtcgagcttagttttcgacactggcttacagcttaacatgttgctattttattgggttctgattgctattttattgggttctgattgctagtttttgctgaatccaaaaaccatgttatttatttaagcgatccaggttattattatgatggtggctaagtttttttttttccaagggtaaattttctggattctccagtgtttctgtggccgaattcactagtgattcagatctgatatcgatgggcccactaactatctatactgtaataatgttgtatagccgccggatagctagctagtttagtcattcagcggcgatgggtaataataaagtgtcatccatccatcaccatgggtggcaacgtgagcaatgacctgattgaacaaattgaaatgaaaagaagaaatatgttatatgtcaacgagatttcctcataatgccactgacgacgtgtgtccaagaaatgtatcagtgatacgtatattcacaatttttttatgacttatactcacaatttgtttttttactacttatactcacaatttgttgtgggtaccataacaatttcgatcgaatatatatcagaaagttgacgaaagtaagctcactcaaaaagttaaatgggctgcggaagctgcgtcaggcccaagttttggctattctatccggtatccacgattttgatggctgagggacatatgttcgcttaagcttggtcacccggtccgggcctagaaggccagcttcaagtttgtacaaaaaagcaggctccggccagaatctcactgactagctaaacagcggccgcatgagcatgcgcttagcagccagaatggccatctggattcagcaggcctagaaggcctcggaccgattaaactttaattcggtccggtaccatgtttaaacctaagggcgcgccattctatcactagctagctgctaattattcccgggcacccagctttcttgtacaaagtggccgttacagaatcactgactagctaatctagcggccgcgccagaagatagaagatatcctggacctgcaagatgtcagcaatgacgattgaaagattcccaggatagccggcggacgtggtggacccagtctaggtgcgatgcttagtcacgcacgatgactctgtcggaaggcatctttactttcggcaaactttaataatactttaggaaaagtattgtacaagttaggtgcagaatcaataatgcacccagctttagtcttgtctactgaattattgtgtcggttgcattattggatgcctgcgtgcaccctaagcaatccccggctctcatctctataagaggagcctttgtattcagttgcaagcatgcaagtcacacactgcaagcttacttctgagcaaaaagagttttgagtgaaataaatttgaagttcccccttacatcttgctcgagaccggtgatcttgtaaggttcccttccctcctcccctcacacccctgttcgtgttccttcggatcggatctcagtggtgatgttagacgtccgcggctgcctacgtagtggcattgccgcccgaaaggtttgtttaggtggggtagatccgaaacaggccggatctggaccatgtccgcggcggggcggcgggacttgatcgcgtagctgtcgtgtgcatttctccctaccagtggcggaatcggcgatgtggacctaagggctaaggcttatctgctgccttgaccatttcgtcgctgacaaaaacaaagtgacaatcatgccgttctctgtttgtttatctggatcgttattacgctgtgaatcctgcgatatgtggctaagtgatttttcttctttttctgggggcagtttagcctttgacccagtcctaggtgtggtcactaggactgtgtagcatgatgagtgaggttgcagcaggctgattgctagtggacgtttttttccccaatttgttaggttttcacgctccaggttgtgcaagtaattttgctagtgattgtgtgatccatcttcaacgttgaaccttgtttttccccctaaaacccccaacaggaaatcttgccccgacttctattgcaaaaattgtaacgcttagcaccctgattgactcaattcctgtcactaggcatgctcggtcaaaagcagatgatttaccacttagaaactgccctgcccctgctttccacatagcatttcgaactttttgactactattgacacccccctaacttgccgaactatttctctcttcagctactatttacctagttataattacataaatgtttgtgtgtatcttgtgcagggatccgccatgggcatcaccgtcacgaacaactcgtccaacccgatcgaggtcgccatcaaccactggggcagcgacggcgacaccagcttcttctccgtcggcaacggcaagcaggagacctgggacaggtccgacagccgcggcttcgtgctctccctgaagaagaacggcgctcagcacccgtactacgtccaggccagcagcaagatcgaggtcgacaacaacgccgtgaaggaccagggtcggctgatcgagccgctctcgtgatacgtatgtgaaaccaatctggatatccctgaaatttgaataaggttatgttgtgagtttatgaagttgtggtctagtgttgtattttgtttttcttaaggataatgcatctgtgtgctttgcttatctaaaatctaaataaagtgggtttctgcattgtgactttgcacaaacttcttttgttaaaatgaataagaaatcacatcaagaaaacagaacaagaccacaacgagaagacttgaaactctctgaaggtatataaacaaacagaacctagtaaattagagtcagcttattcaaaacttttagaccatcatcaagatcattataaacttggagtcaatgttgcggaaaccaatctgctatccccggaatttcaaataaagagactagagaattactttgaacacacttcttttattaaatcggatcacaaaaatccatcaaagacacaacagcaacaaacacaaagagaagacttaaaacactgaagcagtaaacaaacagagagaaataaagtagaatcagcttattcaaagtgtgtaaactatcatcaaaagatacttaaaatctccataaggcgcgccgtctcactagctagctgctaacgttcccgggcaactttattatacaaagttgatagatcgagcgactaattaactagctgtcccacggcctaactagcacttaatcccctagcctaacctaagagcgctaatctaggctagtggtcacttagggctttaaggctagcgtatacgaagttcctattccgaagttcctattctccagaaagtataggaacttctgtacacctgagcctaactaactaggacgtcccgaggtccgattccgggctaattaacacctagctcgccactcgactaattagggactaagcctagcgcttagccgttaaaacctagcacaccctaagcacccttagttaggttcccctcttaattaagccctagtgagcccctaagttaaggggacgctaagagccccctaacctagtattcggctagaggcgaactaggctaaacacctaagcgcacctctttaagctagatcgctagggggctaggctagagcttcgctagattagtctaagggcagctaactaactaggtttaaactacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgaccgtaccccgatgaattaattcggacgtacgttctgaacacagctggatacttacttgggcgattgtcatacatgacatcaacaatgtacccgtttgtgtaaccgtctcttggaggttcgtatgacactagtggttcccctcagcttgcgactagatgttgaggcctaacattttattagagagcaggctagttgcttagatacatgatcttcaggccgttatctgtcagggcaagcgaaaattggccatttatgacgaccaatgccccgcagaagctcccatctttgccgccatagacgccgcgccccccttttggggtgtagaacatccttttgccagatgtggaaaagaagttcgttgtcccattgttggcaatgacgtagtagccggcgaaagtgcgagacccatttgcgctatatataagcctacgatttccgttgcgactattgtcgtaattggatgaactattatcgtagttgctctcagagttgtcgtaatttgatggactattgtcgtaattgcttatggagttgtcgtagttgcttggagaaatgtcgtagttggatggggagtagtcatagggaagacgagcttcatccactaaaacaattggcaggtcagcaagtgcctgccccgatgccatcgcaagtacgaggcttagaaccaccttcaacagatcgcgcatagtcttccccagctctctaacgcttgagttaagccgcgccgcgaagcggcgtcggcttgaacgaattgttagacattatttgccgactaccttggtgatctcgcctttcacgtagtgaacaaattcttccaactgatctgcgcgcgaggccaagcgatcttcttgtccaagataagcctgcctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacagtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactcctgaattaagccgcgccgcgaagcggtgtcggcttgaatgaattgttaggcgtcatcctgtgctcccgagaaccagtaccagtacatcgctgtttcgttcgagacttgaggtctagttttatacgtgaacaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaatcttcatatgacgcctaacgcctggcacagcggatcgcaaacctggcgcggcttttggcacaaaaggcgtgacaggtttgcgaatccgttgctgccacttgttaacccttttgccagatttggtaactataatttatgttagaggcgaagtcttgggtaaaaactggcctaaaattgctggggatttcaggaaagtaaacatcaccttccggctcgatgtctattgtagatatatgtagtgtatctacttgatcgggggatctgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcagggggggggggggggggggttccattgttcattccacggacaaaaacagagaaaggaaacgacagaggccaaaaagctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtcccccccccccccccccctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcggagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccactgtcgactacgccatcatggcgacagcgcctttcctttgggttctctatatcgggcggatcgtggccggcatcaccggggcgactggggcggtagccggcgcttatattgccgatatcactgatggcgatgagcgcgcgcggcacttcggcttcatgagcgcctgtttcgggttcgggatggtcgcgggacctgtgctcggtgggctgatgggcggtttctccccccacgctccgttcttcgccgcggcagccttgaacggcctcaatttcctgacgggctgtttccttttgccggagtcgcacaaaggcgaacgccggccgttacgccgggaggctctcaacccgctcgcttcgttccggtgggcccggggcatgaccgtcgtcgccgccctgatggcggtcttcttcatcatgcaacttgtcggacaggtgccggccgcgctttgggtcattttcggcgaggatcgctttcactgggacgcgaccacgatcggcatttcgcttgccgcatttggcattctgcattcactcgcccaggcaatgatcaccggccctgtagccgcccggctcggcgaaaggcgggcactcatgctcggaatgattgccgacggcacaggctacatcctgcttgccttcgcgacacggggatggatggcgttcccgatcatggtcctgcttgcttcgggtggcatcggaatgccggcgctgcaagcaatgttgtccaggcaggtggatgaggaacgtcaggggcagctgcaaggctcactggcggcgctcaccagcctgacctcgatcgtcggacccctcctcttcacggcgatctatgcggcttctataacaacgtggaacgggtgggcatggattgcaggcgctgccctctacttgctctgcctgccggcgctgcgtcgcgggctttggagcggcgcagggcaacgagccgatcgctgatcgtggaaacgataggcctatgccatgcgggtcaaggcgacttccggcaagctatacgcgccctaggagtgcggttggaacgttggcccagccagatactcccgatcacgagcaggacgccgatgatttgaagcgcactcagcgtctgatccaagaacaaccatcctagcaacacggcggtccccgggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccactcttcattaaccgctatatcgaaaattgcttgcggcttgttagaattgccatgacgtacctcggtgtcacgggtaagattaccgataaactggaactgattatggctcatatcgaaagtctccttgagaaaggagactctagtttagctaaacattggttccgctgtcaagaactttagcggctaaaattttgcgggccgcgaccaaaggtgcgaggggcggcttccgctgtgtacaaccagatatttttcaccaacatccttcgtctgctcgatgagcggggcatgacgaaacatgagctgtcggagagggcaggggtttcaatttcgtttttatcagacttaaccaacggtaaggccaacccctcgttgaaggtgatggaggccattgccgacgccctggaaactcccctacctcttctcctggagtccaccgaccttgaccgcgaggcactcgcggagattgcgggtcatcctttcaagagcagcgtgccgcccggatacgaacgcatcagtgtggttttgccgtcacataaggcgtttatcgtaaagaaatggggcgacgacacccgaaaaaagctgcgtggaaggctctgacgccaagggttagggcttgcacttccttctttagccgctaaaacggccccttctctgcgggccgtcggctcgcgcatcatatcgacatcctcaacggaagccgtgccgcgaatggcatcgggcgggtgcgctttgacagttgttttctatcagaacccctacgtcgtgcggttcgattagctgtttgtcttgcaggctaaacactttcggtatatcgtttgcctgtgcgataatgttgctaatgatttgttgcgtaggggttactgaaaagtgagcgggaaagaagagtttcagaccatcaaggagcgggccaagcgcaagctggaacgcgacatgggtgcggacctgttggccgcgctcaacgacccgaaaaccgttgaagtcatgctcaacgcggacggcaaggtgtggcacgaacgccttggcgagccgatgcggtacatctgcgacatgcggcccagccagtcgcaggcgattatagaaacggtggccggattccacggcaaagaggtcacgcggcattcgcccatcctggaaggcgagttccccttggatggcagccgctttgccggccaattgccgccggtcgtggccgcgccaacctttgcgatccgcaagcgcgcggtcgccatcttcacgctggaacagtacgtcgaggcgggcatcatgacccgcgagcaatacgaggtcattaaaagcgccgtcgcggcgcatcgaaacatcctcgtcattggcggtactggctcgggcaagaccacgctcgtcaacgcgatcatcaatgaaatggtcgccttcaacccgtctgagcgcgtcgtcatcatcgaggacaccggcgaaatccagtgcgccgcagagaacgccgtccaataccacaccagcatcgacgtctcgatgacgctgctgctcaagacaacgctgcgtatgcgccccgaccgcatcctggtcggtgaggtacgtggccccgaagcccttgatctgttgatggcctggaacaccgggcatgaaggaggtgccgccaccctgcacgcaaacaaccccaaagcgggcctgagccggctcgccatgcttatcagcatgcacccggattcaccgaaacccattgagccgctgattggcgaggcggttcatgtggtcgtccatatcgccaggacccctagcggccgtcgagtgcaagaaattctcgaagttcttggttacgagaacggccagtacatcaccaaaaccctgtaaggagtatttccaatgacaacggctgttccgttccgtctgaccatgaatcgcggcattttgttctaccttgccgtgttcttcgttctcgctctcgcgttatccgcgcatccggcgatggcctcggaaggcaccggcggcagcttgccatatgagagctggctgacgaacctgcgcaactccgtaaccggcccggtggccttcgcgctgtccatcatcggcatcgtcgtcgccggcggcgtgctgatcttcggcggcgaactcaacgccttcttccgaaccctgatcttcctggttctggtgatggcgctgctggtcggcgcgcagaacgtgatgagcaccttcttcggtcgtggtgccgaaatcgcggccctcggcaacggggcgctgcaccaggtgcaagtcgcggcggcggatgccgtgcgtgcggtagcggctggacggctcgcctaatcatggctctgcgcacgatccccatccgtcgcgcaggcaaccgagaaaacctgttcatgggtggtgatcgtgaactggtgatgttctcgggcctgatggcgtttgcgctgattttcagcgcccaagagctgcgggccaccgtggtcggtctgatcctgtggttcggggcgctctatgcgttccgaatcatggcgaaggccgatccgaagatgcggttcgtgtacctgcgtcaccgccggtacaagccgtattacccggcccgctcgaccccgttccgcgagaacaccaatagccaagggaagcaataccgatgatccaagcaattgcgattgcaatcgcgggcctcggcgcgcttctgttgttcatcctctttgcccgcatccgcgcggtcgatgccgaactgaaactgaaaaagcatcgttccaaggacgccggcctggccgatctgctcaactacgccgctgtcgtcgatgacggcgtaatcgtgggcaagaacggcagctttatggctgcctggctgtacaagggcgatgacaacgcaagcagcaccgaccagcagcgcgaagtagtgtccgcccgcatcaaccaggccctcgcgggcctgggaagtgggtggatgatccatgtggacgccgtgcggcgtcctgctccgaactacgcggagcggggcctgtcggcgttccctgaccgtctgacggcagcgattgaagaagagcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctacaccgacgagctggccgagtgggttgaatcccgcgcggccaagaagcgccggcgtgatgaggctgcggttgcgttcctggcggtgagggcggatgtcgaggcggcgttagcgtccggctatgcgctcgtcaccatttgggagcacatgcgggaaacggggaaggtcaagttctcctacgagacgttccgctcgcacgccaggcggcacatcaaggccaagcccgccgatgtgcccgcaccgcaggccaaggctgcggaacccgcgccggcacccaagacgccggagccacggcggccgaagcaggggggcaaggctgaaaagccggcccccgctgcggccccgaccggcttcaccttcaacccaacaccggacaaaaaggatctactgtaatggcgaaaattcacatggttttgcagggcaagggcggggtcggcaagtcggccatcgccgcgatcattgcgcagtacaagatggacaaggggcagacacccttgtgcatcgacaccgacccggtgaacgcgacgttcgagggctacaaggccctgaacgtccgccggctgaacatcatggccggcgacgaaattaactcgcgcaacttcgacaccctggtcgagctgattgcgccgaccaaggatgacgtggtgatcgacaacggtgccagctcgttcgtgcctctgtcgcattacctcatcagcaaccaggtgccggctctgctgcaagaaatggggcatgagctggtcatccataccgtcgtcaccggcggccaggctctcctggacacggtgagcggcttcgcccagctcgccagccagttcccggccgaagcgcttttcgtggtctggctgaacccgtattgggggcctatcgagcatgagggcaagagctttgagcagatgaaggcgtacacggccaacaaggcccgcgtgtcgtccatcatccagattccggccctcaaggaagaaacctacggccgcgatttcagcgacatgctgcaagagcggctgacgttcgaccaggcgctggccgatgaatcgctcacgatcatgacgcggcaacgcctcaagatcgtgcggcgcggcctgtttgaacagctcgacgcggcggccgtgctatgagcgaccagattgaagagctgatccgggagattgcggccaagcacggcatcgccgtcggccgcgacgacccggtgctgatcctgcataccatcaacgcccggctcatggccgacagtgcggccaagcaagaggaaatccttgccgcgttcaaggaagagctggaagggatcgcccatcgttggggcgaggacgccaaggccaaagcggagcggatgctgaacgcggccctggcggccagcaaggacgcaatggcgaaggtaatgaaggacagcgccgcgcaggcggccgaagcgatccgcagggaaatcgacgacggccttggccgccagctcgcggccaaggtcgcggacgcgcggcgcgtggcgatgatgaacatgatcgccggcggcatggtgttgttcgcggccgccctggtggtgtgggcctcgttatgaatcgcagaggcgcagatgaaaaagcccggcgttgccgggctttgtttttgcgttagctgggcttgtttgacaggcccaagctctgactgcgcccgcgctcgcgctcctgggcctgtttcttctcctgctcctgcttgcgcatcagggcctggtgccgtcgggctgcttcacgcatcgaatcccagtcgccggccagctcgggatgctccgcgcgcatcttgcgcgtcgccagttcctcgatcttgggcgcgtgaatgcccatgccttccttgatttcgcgcaccatgtccagccgcgtgtgcagggtctgcaagcgggcttgctgttgggcctgctgctgctgccaggcggcctttgtacgcggcagggacagcaagccgggggcattggactgtagctgctgcaaacgcgcctgctgacggtctacgagctgttctaggcggtcctcgatgcgctccacctggtcatgctttgcctgcacgtagagcgcaagggtctgctggtaggtctgctcgatgggcgcggattctaagagggcctgctgttccgtctcggcctcctgggccgcctgtagcaaatcctcgccgctgttgccgctggactgctttactgccggggactgctgttgccctgctcgcgccgtcgtcgcagttcggcttgcccccactcgattgactgcttcatttcgagccgcagcgatgcgatctcggattgcgtcaacggacggggcagcgcggaggtgtccggcttctccttgggtgagtcggtcgatgccatagccaaaggtttccttccaaaatgcgtccattgctggaccgtgtttctcattgatgcccgcaagcatcttcggcttgaccgccaggtcaagcgcgccttcatgggcggtcatgacggacgccgccatgaccttgccgccgttgttctcgatgtagccgcgtaatgaggcaatggtgccgcccatcgtcagcgtgtcatcgacaacgatgtacttctggccggggatcacctccccctcgaaagtcgggttgaacgccaggcgatgatctgaaccggctccggttcgggcgaccttctcccgctgcacaatgtccgtttcgacctcaaggccaaggcggtcggccagaacgaccgccatcatggccggaatcttgttgttccccgccgcctcgacggcgaggactggaacgatgcggggcttgtcgtcgccgatcagcgtcttgagctgggcaacagtgtcgtccgaaatcaggcgctcgaccaaattaagcgccgcttccgcgtcgccctgcttcgcagcctggtattcaggctcgttggtcaaagaaccaaggtcgccgttgcgaaccaccttcgggaagtctccccacggtgcgcgctcggctctgctgtagctgctcaagacgcctccctttttagccgctaaaactctaacgagtgcgcccgcgactcaacttgacgctttcggcacttacctgtgccttgccacttgcgtcataggtgatgcttttcgcactcccgatttcaggtactttatcgaaatctgaccgggcgtgcattacaaagttcttccccacctgttggtaaatgctgccgctatctgcgtggacgatgctgccgtcgtggcgctgcgacttatcggccttttgggccatatagatgttgtaaatgccaggtttcagggccccggctttatctaccttctggttcgtccatgcgccttggttctcggtctggacaattctttgcccattcatgaccaggaggcggtgtttcattgggtgactcctgacggttgcctctggtgttaaacgtgtcctggtcgcttgccggctaaaaaaaagccgacctcggcagttcgaggccggctttccctagagccgggcgcgtcaaggttgttccatctattttagtgaactgcgttcgatttatcagttactttcctcccgctttgtgtttcctcccactcgtttccgcgtctagccgacccctcaacatagcggcctcttcttgggctgcctttgcctcttgccgcgcttcgtcacgctcggcttgcaccgtcgtaaagcgctcggcctgcctggccgcctcttgcgccgccaacttcctttgctcctggtgggcctcggcgtcggcctgcgccttcgctttcaccgctgccaactccgtgcgcaaactctccgcttcgcgcctggtggcgtcgcgctcgccgcgaagcgcctgcatttcctggttggccgcgtccagggtcttgcggctctcttctttgaatgcgcgggcgtcctggtgagcgtagtccagctcggcgcgcagctcctgcgctcgacgctccacctcgtcggcccgctgcgtcgccagcgcggcccgctgctcggctcctgccagggcggtgcgtgcttcggccagggcttgccgctggcgtgcggccagctcggccgcctcggcggcctgctgctctagcaatgtaacgcgcgcctgggcttcttccagctcgcgggcctgcgcctcgaaggcgtcggccagctccccgcgcacggcttccaactcgttgcgctcacgatcccagccggcttgcgctgcctgcaacgattcattggcaagggcctgggcggcttgccagagggcggccacggcctggttgccggcctgctgcaccgcgtccggcacctggactgccagcggggcggcctgcgccgtgcgctggcgtcgccattcgcgcatgccggcgctggcgtcgttcatgttgacgcgggcggccttacgcactgcatccacggtcgggaagttctcccggtcgccttgctcgaacagctcgtccgcagccgcaaaaatgcggtcgcgcgtctctttgttcagttccatgttggctccggtaattggtaagaataataatactcttacctaccttatcagcgcaagagtttagctgaacagttctcgacttaacggcaggttttttagcggctgaagggcaggcaaaaaaagccccgcacggtcggcgggggcaaagggtcagcgggaaggggattagcgggcgtcgggcttcttcatgcgtcggggccgcgcttcttgggatggagcacgacgaagcgcgcacgcgcatcgtcctcggccctatcggcccgcgtcgcggtcaggaacttgtcgcgcgctaggtcctccctggtgggcaccaggggcatgaactcggcctgctcgatgtaggtccactccatgaccgcatcgcagtcgaggccgcgttccttcaccgtctcttgcaggtcgcggtacgcccgctcgttgagcggctggtaacgggccaattggtcgtaaatggctgtcggccatgagcggcctttcctgttgagccagcagccgacgacgaagccggcaatgcaggcccctggcacaaccaggccgacgccgggggcaggggatggcagcagctcgccaaccaggaaccccgccgcgatgatgccgatgccggtcaaccagcccttgaaactatccggccccgaaacacccctgcgcattgcctggatgctgcgccggatagcttgcaacatcaggagccgtttcttttgttcgtcagtcatggtccgccctcaccagttgttcgtatcggtgtcggacgaactgaaatcgcaagagctgccggtatcggtccagccgctgtccgtgtcgctgctgccgaagcacggcgaggggtccgcgaacgccgcagacggcgtatccggccgcagcgcatcgcccagcatggccccggtcagcgagccgccggccaggtagcccagcatggtgctgttggtcgccccggccaccagggccgacgtgacgaaatcgccgtcattccctctggattgttcgctgctcggcggggcagtgcgccgcgccggcggcgtcgtggatggctcgggttggctggcctgcgacggccggcgaaaggtgcgcagcagctcgttatcgaccggctgcggcgtcggggccgccgccttgcgctgcggtcggtgttccttcttcggctcgcgcagcttgaacagcatgatcgcggaaaccagcagcaacgccgcgcctacgcctcccgcgatgtagaacagcatcggattcattcttcggtcctccttgtagcggaaccgttgtctgtgcggcgcgggtggcccgcgccgctgtctttggggatcagccctcgatgagcgcgaccagtttcacgtcggcaaggttcgcctcgaactcctggccgtcgtcctcgtacttcaaccaggcatagccttccgccggcggccgacggttgaggataaggcgggcagggcgctcgtcgtgctcgacctggacgatggcctttttcagcttgtccgggtccggctccttcgcgcccttttccttggcgtccttaccgtcctggtcgccgtcctcgccgtcctggccgtcgccggcctccgcgtcacgctcggcatcagtctggccgttgaaggcatcgacggtgttgggatcgcggcccttctcgtccaggaactcgcgcagcagcttgaccgtgccgcgcgtgatttcctgggtgtcgtcgtcaagccacgcctcgacttcctccgggcgcttcttgaaggccgtcaccagctcgttcaccacggtcacgtcgcgcacgcggccggtgttgaacgcatcggcgatcttctccggcaggtccagcagcgtgacgtgctgggtgatgaacgccggcgacttgccgatttccttggcgatatcgcctttcttcttgcccttcgccagctcgcggccaatgaagtcggcaatttcgcgcggggtcagctcgttgcgttgcaggttctcgataacctggtcggcttcgttgtagtcgttgtcgatgaacgccgggatggacttcttgccggcccacttcgagccacggtagcggcgggcgccgtgattgatgatatagcggcccggctgctcctggttctcgcgcaccgaaatgggtgacttcaccccgcgctctttgatcgtggcaccgatttccgcgatgctctccggggaaaagccggggttgtcggccgtccgcggctgatgcggatcttcgtcgatcaggtccaggtccagctcgatagggccggaaccgccctgagacgccgcaggagcgtccaggaggctcgacaggtcgccgatgctatccaaccccaggccggacggctgcgccgcgcctgcggcttcctgagcggccgcagcggtgtttttcttggtggtcttggcttgagccgcagtcattgggaaatctccatcttcgtgaacacgtaatcagccagggcgcgaacctctttcgatgccttgcgcgcggccgttttcttgatcttccagaccggcacaccggatgcgagggcatcggcgatgctgctgcgcaggccaacggtggccggaatcatcatcttggggtacgcggccagcagctcggcttggtggcgcgcgtggcgcggattccgcgcatcgaccttgctgggcaccatgccaaggaattgcagcttggcgttcttctggcgcacgttcgcaatggtcgtgaccatcttcttgatgccctggatgctgtacgcctcaagctcgatgggggacagcacatagtcggccgcgaagagggcggccgccaggccgacgccaagggtcggggccgtgtcgatcaggcacacgtcgaagccttggttcgccagggccttgatgttcgccccgaacagctcgcgggcgtcgtccagcgacagccgttcggcgttcgccagtaccgggttggactcgatgagggcgaggcgcgcggcctggccgtcgccggctgcgggtgcggtttcggtccagccgccggcagggacagcgccgaacagcttgcttgcatgcaggccggtagcaaagtccttgagcgtgtaggacgcattgccctgggggtccaggtcgatcacggcaacccgcaagccgcgctcgaaaaagtcgaaggcaagatgcacaagggtcgaagtcttgccgacgccgcctttctggttggccgtgaccaaagttttcatcgtttggtttcctgttttttcttggcgtccgcttcccacttccggacgatgtacgcctgatgttccggcagaaccgccgttacccgcgcgtacccctcgggcaagttcttgtcctcgaacgcggcccacacgcgatgcaccgcttgcgacactgcgcccctggtcagtcccagcgacgttgcgaacgtcgcctgtggcttcccatcgactaagacgccccgcgctatctcgatggtctgctgccccacttccagcccctggatcgcctcctggaactggctttcggtaagccgtttcttcatggataacacccataatttgctccgcgccttggttgaacatagcggtgacagccgccagcacatgagagaagtttagctaaacatttctcgcacgtcaacacctttagccgctaaaactcgtccttggcgtaacaaaacaaaagcccggaaaccgggctttcgtctcttgccgcttatggctctgcacccggctccatcaccaacaggtcgcgcacgcgcttcactcggttgcggatcgacactgccagcccaacaaagccggttgccgccgccgccaggatcgcgccgatgatgccggccacaccggccatcgcccaccaggtcgccgccttccggttccattcctgctggtactgcttcgcaatgctggacctcggctcaccataggctgaccgctcgatggcgtatgccgcttctccccttggcgtaaaacccagcgccgcaggcggcattgccatgctgcccgccgctttcccgaccacgacgcgcgcaccaggcttgcggtccagaccttcggccacggcgagctgcgcaaggacataatcagccgccgacttggctccacgcgcctcgatcagctcttgcactcgcgcgaaatccttggcctccacggccgccatgaatcgcgcacgcggcgaaggctccgcagggccggcgtcgtgatcgccgccgagaatgcccttcaccaagttcgacgacacgaaaatcatgctgacggctatcaccatcatgcagacggatcgcacgaacccgctgaattgaacacgagcacggcacccgcgaccactatgccaagaatgcccaaggtaaaaattgccggccccgccatgaagtccgtgaatgccccgacggccgaagtgaagggcaggccgccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaaggcgctggaagccccgtagcgacgcggagaggggcgagacaagccaagggcgcaggctcgatgcgcagcacgacatagccggttctcgcaaggacgagaatttccctgcggtgcccctcaagtgtcaatgaaagtttccaacgcgagccattcgcgagagccttgagtccacgctagatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgactctagagctcgttcctcgaggcctcgaggcctcgaggaacggtacctgcggggaagcttacaataatgtgtgttgttaagtcttgttgcctgtcatcgtctgactgactttcgtcataaatcccggcctccgtaacccagctttgggcaagctcacggatttgatccggcggaacgggaatatcgagatgccgggctgaacgctgcagttccagctttccctttcgggacaggtactccagctgattgattatctgctgaagggtcttggttccacctcctggcacaatgcgaatgattacttgagcgcgatcgggcatccaattttctcccgtcaggtgcgtggtcaagtgctacaaggcacctttcagtaacgagcgaccgtcgatccgtcgccgggatacggacaaaatggagcgcagtagtccatcgagggcggcgaaagcctcgccaaaagcaatacgttcatctcgcacagcctccagatccgatcgagggtcttcggcgtaggcagatagaagcatggatacattgcttgagagtattccgatggactgaagtatggcttccatcttttctcgtgtgtctgcatctatttcgagaaagcccccgatgcggcgcaccgcaacgcgaattgccatactatccgaaagtcccagcaggcgcgcttgataggaaaaggtttcatactcggccgatcgcagacgggcactcacgaccttgaacccttcaactttcagggatcgatgctggttgatggtagtctcactcgacgtggctctggtgtgttttgacatagcttcctccaaagaaagcggaaggtctggatactccagcacgaaatgtgcccgggtagacggatggaagtctagccctgctcaatatgaaatcaacagtacatttacagtcaatactgaatatacttgctacatttgcaattgtcttataacgaatgtgaaataaaaatagtgtaacaacgcttttactcatcgataatcacaaaaacatttatacgaacaaaaatacaaatgcactccggtttcacaggataggcgggatcagaatatgcaacttttgacgttttgttctttcaaagggggtgctggcaaaaccaccgcactcatgggcctttgcgctgctttggcaaatgacggtaaacgagtggccctctttgatgccgacgaaaaccggcctctgacgcgatggagagaaaacgccttacaaagcagtactgggatcctcgctgtgaagtctattccgccgacgaaatgccccttcttgaagcagcctatgaaaatgccgagctcgaaggatttgattatgcgttggccgatacgcgtggcggctcgagcgagctcaacaacacaatcatcgctagctcaaacctgcttctgatccccaccatgctaacgccgctcgacatcgatgaggcactatctacctaccgctacgtcatcgagctgctgttgagtgaaaatttggcaattcctacagctgttttgcgccaacgcgtcccggtcggccgattgacaacatcgcaacgcaggatgtcagagacgctagagagccttccagttgtaccgtctcccatgcatgaaagagatgcatttgccgcgatgaaagaacgcggcatgttgcatcttacattactaaacacgggaactgatccgacgatgcgcctcatagagaggaatcttcggattgcgatggaggaagtcgtggtcatttcgaaactgatcagcaaaatcttggaggcttgaagatggcaattcgcaagcccgcattgtcggtcggcgaagcacggcggcttgctggtgctcgacccgagatccaccatcccaacccgacacttgttccccagaagctggacctccagcacttgcctgaaaaagccgacgagaaagaccagcaacgtgagcctctcgtcgccgatcacatttacagtcccgatcgacaacttaagctaactgtggatgcccttagtccacctccgtccccgaaaaagctccaggtttttctttcagcgcgaccgcccgcgcctcaagtgtcgaaaacatatgacaacctcgttcggcaatacagtccctcgaagtcgctacaaatgattttaaggcgcgcgttggacgatttcgaaagcatgctggcagatggatcatttcgcgtggccccgaaaagttatccgatcccttcaactacagaaaaatccgttctcgttcagacctcacgcatgttcccggttgcgttgctcgaggtcgctcgaagtcattttgatccgttggggttggagaccgctcgagctttcggccacaagctggctaccgccgcgctcgcgtcattctttgctggagagaagccatcgagcaattggtgaagagggacctatcggaacccctcaccaaatattgagtgtaggtttgaggccgctggccgcgtcctcagtcaccttttgagccagataattaagagccaaatgcaattggctcaggctgccatcgtccccccgtgcgaaacctgcacgtccgcgtcaaagaaataaccggcacctcttgctgtttttatcagttgagggcttgacggatccgcctcaagtttgcggcgcagccgcaaaatgagaacatctatactcctgtcgtaaacctcctcgtcgcgtactcgactggcaatgagaagttgctcgcgcgatagaacgtcgcggggtttctctaaaaacgcgaggagaagattgaactcacctgccgtaagtttcacctcaccgccagcttcggacatcaagcgacgttgcctgagattaagtgtccagtcagtaaaacaaaaagaccgtcggtctttggagcggacaacgttggggcgcacgcgcaaggcaacccgaatgcgtgcaagaaactctctcgtactaaacggcttagcgataaaatcacttgctcctagctcgagtgcaacaactttatccgtctcctcaaggcggtcgccactgataattatgattggaatatcagactttgccgccagatttcgaacgatctcaagcccatcttcacgacctaaatttagatcaacaaccacgacatcgaccgtcgcggaagagagtactctagtgaactgggtgctgtcggctaccgcggtcactttgaaggcgtggatcgtaaggtattcgataataagatgccgcatagcgacatcgtcatcgataagaagaacgtgtttcaacggctcacctttcaatctaaaatctgaacccttgttcacagcgcttgagaaattttcacgtgaaggatgtacaatcatctccagctaaatgggcagttcgtcagaattgcggctgaccgcggatgacgaaaatgcgaaccaagtatttcaattttatgacaaaagttctcaatcgttgttacaagtgaaacgcttcgaggttacagctactattgattaaggagatcgcctatggtctcgccccggcgtcgtgcgtccgccgcgagccagatctcgcctacttcataaacgtcctcataggcacggaatggaatgatgacatcgatcgccgtagagagcatgtcaatcagtgtgcgatcttccaagctagcaccttgggcgctacttttgacaagggaaaacagtttcttgaatccttggattggattcgcgccgtgtattgttgaaatcgatcccggatgtcccgagacgacttcactcagataagcccatgctgcatcgtcgcgcatctcgccaagcaatatccggtccggccgcatacgcagacttgcttggagcaagtgctcggcgctcacagcacccagcccagcaccgttcttggagtagagtagtctaacatgattatcgtgtggaatgacgagttcgagcgtatcttctatggtgattagcctttcctggggggggatggcgctgatcaaggtcttgctcattgttgtcttgccgcttccggtagggccacatagcaacatcgtcagtcggctgacgacgcatgcgtgcagaaacgcttccaaatccccgttgtcaaaatgctgaaggatagcttcatcatcctgattttggcgtttccttcgtgtctgccactggttccacctcgaagcatcataacgggaggagacttctttaagaccagaaacacgcgagcttggccgtcgaatggtcaagctgacggtgcccgagggaacggtcggcggcagacagatttgtagtcgttcaccaccaggaagttcagtggcgcagagggggttacgtggtccgacatcctgctttctcagcgcgcccgctaaaatagcgatatcttcaagatcatcataagagacgggcaaaggcatcttggtaaaaatgccggcttggcgcacaaatgcctctccaggtcgattgatcgcaatttcttcagtcttcgggtcatcgagccattccaaaatcggcttcagaagaaagcgtagttgcggatccacttccatttacaatgtatcctatctctaagcggaaatttgaattcattaagagcggcggttcctcccccgcgtggcgccgccagtcaggcggagctggtaaacaccaaagaaatcgaggtcccgtgctacgaaaatggaaacggtgtcaccctgattcttcttcagggttggcggtatgttgatggttgccttaagggctgtctcagttgtctgctcaccgttattttgaaagctgttgaagctcatcccgccacccgagctgccggcgtaggtgctagctgcctggaaggcgccttgaacaacactcaagagcatagctccgctaaaacgctgccagaagtggctgtcgaccgagcccggcaatcctgagcgaccgagttcgtccgcgcttggcgatgttaacgagatcatcgcatggtcaggtgtctcggcgcgatcccacaacacaaaaacgcgcccatctccctgttgcaagccacgctgtatttcgccaacaacggtggtgccacgatcaagaagcacgatattgttcgttgttccacgaatatcctgaggcaagacacactttacatagcctgccaaatttgtgtcgattgcggtttgcaagatgcacggaattattgtcccttgcgttaccataaaatcggggtgcggcaagagcgtggcgctgctgggctgcagctcggtgggtttcatacgtatcgacaaatcgttctcgccggacacttcgccattcggcaaggagttgtcgtcacgcttgccttcttgtcttcggcccgtgtcgccctgaatggcgcgtttgctgaccccttgatcgccgctgctatatgcaaaaatcggtgtttcttccggccgtggctcatgccgctccggttcgcccctcggcggtagaggagcagcaggctgaacagcctcttgaaccgctggaggatccggcggcacctcaatcggagctggatgaaatggcttggtgtttgttgcgatcaaagttgacggcgatgcgttctcattcaccttcttttggcgcccacctagccaaatgaggcttaatgataacgcgagaacgacacctccgacgatcaatttctgagaccccgaaagacgccggcgatgtttgtcggagaccagggatccagatgcatcaacctcatgtgccgcttgctgactatcgttattcatcccttcgcccccttcaggacgcgtttcacatcgggcctcaccgtgcccgtttgcggcctttggccaacgggatcgtaagcggtgttccagatacatagtactgtgtggccatccctcagacgccaacctcgggaaaccgaagaaatctcgacatcgctccctttaactgaatagttggcaacagcttccttgccatcaggattgatggtgtagatggagggtatgcgtacattgcccggaaagtggaataccgtcgtaaatccattgtcgaagacttcgagtggcaacagcgaacgatcgccttgggcgacgtagtgccaattactgtccgccgcaccaagggctgtgacaggctgatccaataaattctcagctttccgttgatattgtgcttccgcgtgtagtctgtccacaacagccttctgttgtgcctcccttcgccgagccgccgcatcgtcggcggggtaggcgaattggacgctgtaatagagatcgggctgctctttatcgaggtgggacagagtcttggaacttatactgaaaacataacggcgcatcccggagtcgcttgcggttagcacgattactggctgaggcgtgaggacctggcttgccttgaaaaatagataatttccccgcggtagggctgctagatctttgctatttgaaacggcaaccgctgtcaccgtttcgttcgtggcgaatgttacgaccaaagtagctccaaccgccgtcgagaggcgcaccacttgatcgggattgtaagccaaataacgcatgcgcggatctagcttgcccgccattggagtgtcttcagcctccgcaccagtcgcagcggcaaataaacatgctaaaatgaaaagtgcttttctgatcatggttcgctgtggcctacgtttgaaacggtatcttccgatgtctgataggaggtgacaaccagacctgccgggttggttagtctcaatctgccgggcaagctggtcaccttttcgtagcgaactgtcgcggtccacgtactcaccacaggcattttgccgtcaacgacgagggtccttttatagcgaatttgctgcgtgcttggagttacatcatttgaagcgatgtgctcgacctccaccctgccgcgtttgccaagaatgacttgaggcgaactgggattgggatagttgaagaattgctggtaatcctggcgcactgttggggcactgaagttcgataccaggtcgtaggcgtactgagcggtgtcggcatcataactctcgcgcaggcgaacgtactcccacaatgaggcgttaacgacggcctcctcttgagttgcaggcaatcgcgagacagacacctcgctgtcaacggtgccgtccggccgtatccatagatatacgggcacaagcctgctcaacggcaccattgtggctatagcgaacgcttgagcaacatttcccaaaatcgcgatagctgcgacagctgcaatgagtttggagagacgtcgcgccgatttcgctcgcgcggtttgaaaggcttctacttccttatagtgctcggcaaggctttcgcgcgccactagcatggcatattcaggccccgtcatagcgtccacccgaattgccgagctgaagatctgacggagtaggctgccatcgccccacattcagcgggaagatcgggcctttgcagctcgctaatgtgtcgtttgtctggcagccgctcaaagcgacaactaggcacagcaggcaatacttcatagaattctccattgaggcgaatttttgcgcgacctagcctcgctcaacctgagcgaagcgacggtacaagctgctggcagattgggttgcgccgctccagtaactgcctccaatgttgccggcgatcgccggcaaagcgacaatgagcgcatcccctgtcagaaaaaacatatcgagttcgtaaagaccaatgatcttggccgcggtcgtaccggcgaaggtgattacaccaagcataagggtgagcgcagtcgcttcggttaggatgacgatcgttgccacgaggtttaagaggagaagcaagagaccgtaggtgataagttgcccgatccacttagctgcgatgtcccgcgtgcgatcaaaaatatatccgacgaggatcagaggcccgatcgcgagaagcactttcgtgagaattccaacggcgtcgtaaactccgaaggcagaccagagcgtgccgtaaaggacccactgtgccccttggaaagcaaggatgtcctggtcgttcatcggaccgatttcggatgcgattttctgaaaaacggcctgggtcacggcgaacattgtatccaactgtgccggaacagtctgcagaggcaagccggttacactaaactgctgaacaaagtttgggaccgtcttttcgaagatggaaaccacatagtcttggtagttagcctgcccaacaattagagcaacaacgatggtgaccgtgatcacccgagtgataccgctacgggtatcgacttcgccgcgtatgactaaaataccctgaacaataatccaaagagtgacacaggcgatcaatggcgcactcaccgcctcctggatagtctcaagcatcgagtccaagcctgtcgtgaaggctacatcgaagatcgtatgaatggccgtaaacggcgccggaatcgtgaaattcatcgattggacctgaacttgactggtttgtcgcataatgttggataaaatgagctcgcattcggcgaggatgcgggcggatgaacaaatcgcccagccttaggggagggcaccaaagatgacagcggtcttttgatgctccttgcgttgagcggccgcctcttccgcctcgtgaaggccggcctgcgcggtagtcatcgttaataggcttgtcgcctgtacattttgaatcattgcgtcatggatctgcttgagaagcaaaccattggtcacggttgcctgcatgatattgcgagatcgggaaagctgagcagacgtatcagcattcgccgtcaagcgtttgtccatcgtttccagattgtcagccgcaatgccagcgctgtttgcggaaccggtgatctgcgatcgcaacaggtccgcttcagcatcactacccacgactgcacgatctgtatcgctggtgatcgcacgtgccgtggtcgacattggcattcgcggcgaaaacatttcattgtctaggtccttcgtcgaaggatactgatttttctggttgagcgaagtcagtagtccagtaacgccgtaggccgacgtcaacatcgtaaccatcgctatagtctgagtgagattctccgcagtcgcgagcgcagtcgcgagcgtctcagcctccgttgccgggtcgctaacaacaaactgcgcccgcgcgggctgaatatatagaaagctgcaggtcaaaactgttgcaataagttgcgtcgtcttcatcgtttcctaccttatcaatcttctgcctcgtggtgacgggccatgaattcgctgagccagccagatgagttgccttcttgtgcctcgcgtagtcgagttgcaaagcgcaccgtgttggcacgccccgaaagcacggcgacatattcacgcatatcccgcagatcaaattcgcagatgacgcttccactttctcgtttaagaagaaacttacggctgccgaccgtcatgtcttcacggatcgcctgaaattccttttcggtacatttcagtccatcgacataagccgatcgatctgcggttggtgatggatagaaaatcttcgtcatacattgcgcaaccaagctggctcctagcggcgattccagaacatgctctggttgctgcgttgccagtattagcatcccgttgttttttcgaacggtcaggaggaatttgtcgacgacagtcgaaaatttagggtttaacaaataggcgcgaaactcatcgcagctcatcacaaaacggcggccgtcgatcatggctccaatccgatgcaggagatatgctgcagcgggagcgcatacttcctcgtattcgagaagatgcgtcatgtcgaagccggtaatcgacggatctaactttacttcgtcaacttcgccgtcaaatgcccagccaagcgcatggccccggcaccagcgttggagccgcgctcctgcgccttcggcgggcccatgcaacaaaaattcacgtaaccccgcgattgaacgcatttgtggatcaaacgagagctgacgatggataccacggaccagacggcggttctcttccggagaaatcccaccccgaccatcactctcgatgagagccacgatccattcgcgcagaaaatcgtgtgaggctgctgtgttttctaggccacgcaacggcgccaacccgctgggtgtgcctctgtgaagtgccaaatatgttcctcctgtggcgcgaaccagcaattcgccaccccggtccttgtcaaagaacacgaccgtacctgcacggtcgaccatgctctgttcgagcatggctagaacaaacatcatgagcgtcgtcttacccctcccgataggcccgaatattgccgtcatgccaacatcgtgctcatgcgggatatagtcgaaaggcgttccgccattggtacgaaatcgggcaatcgcgttgccccagtggcctgagctggcgccctctggaaagttttcgaaagagacaaaccctgcgaaattgcgtgaagtgattgcgccagggcgtgtgcgccacttaaaattccccggcaattgggaccaataggccgcttccataccaataccttcttggacaaccacggcacctgcatccgccattcgtgtccgagcccgcgcgcccctgtccccaagactattgagatcgtctgcatagacgcaaaggctcaaatgatgtgagcccataacgaattcgttgctcgcaagtgcgtcctcagcctcggataatttgccgatttgagtcacggctttatcgccggaactcagcatctggctcgatttgaggctaagtttcgcgtgcgcttgcgggcgagtcaggaacgaaaaactctgcgtgagaacaagtggaaaatcgagggatagcagcgcgttgagcatgcccggccgtgtttttgcagggtattcgcgaaacgaatagatggatccaacgtaactgtcttttggcgttctgatctcgagtcctcgcttgccgcaaatgactctgtcggtataaatcgaagcgccgagtgagccgctgacgaccggaaccggtgtgaaccgaccagtcatgatcaaccgtagcgcttcgccaatttcggtgaagagcacaccctgcttctcgcggatgccaagacgatgcaggccatacgctttaagagagccagcgacaacatgccaaagatcttccatgttcctgatctggcccgtgagatcgttttccctttttccgcttagcttggtgaacctcctctttaccttccctaaagccgcctgtgggtagacaatcaacgtaaggaagtgttcattgcggaggagttggccggagagcacgcgctgttcaaaagcttcgttcaggctagcggcgaaaacactacggaagtgtcgcggcgccgatgatggcacgtcggcatgacgtacgaggtgagcatatattgacacatgatcatcagcgatattgcgcaacagcgtgttgaacgcacgacaacgcgcattgcgcatttcagtttcctcaagctcgaatgcaacgccatcaattctcgcaatggtcatgatcgatccgtcttcaagaaggacgatatggtcgctgaggtggccaatataagggagatagatctcaccggatctttcggtcgttccactcgcgccgagcatcacaccattcctctccctcgtgggggaaccctgattggatttgggctaacagtagcgcccccccaaactgcactatcaatgcttcttcccgcggtccgcaaaaatagcaggacgacgctcgccgcattgtagtctcgctccacgatgagccgggctgcaaaccataacggcacgagaacgacttcgtagagcgggttctgaacgataacgatgacaaagccggcgaacatcatgaataaccctgccaatgtcagtggcaccccaagaaacaatgcgggccgtgtggctgcgaggtaaagggtcgattcttccaaacgatcagccatcaactaccgccagtgagcgtttggccgaggaagctcgccccaaacatgataacaatgccgccgacgacgccggcaaccagcccaagcgaagcccgcccgaacatccaggagatcccgatagcgacaatgccgagaacagcgagtgactggccgaacggaccaaggataaacgtgcatatattgttaaccattgtggcggggtcagtgccgccacccgcagattgcgctgcggcgggtccggatgaggaaatgctccatgcaattgcaccgcacaagcttggggcgcagctcgatatcacgcgcatcatcgcattcgagagcgagaggcgatttagatgtaaacggtatctctcaaagcatcgcatcaatgcgcacctccttagtataagtcgaataagacttgattgtcgtctgcggatttgccgttgtcctggtgtggcggtggcggagcgattaaaccgccagcgccatcctcctgcgagcggcgctgatatgacccccaaacatcccacgtctcttcggattttagcgcctcgtgatcgtcttttggaggctcgattaacgcgggcaccagcgattgagcagctgtttcaacttttcgcacgtagccgtttgcaaaaccgccgatgaaattaccggtgttgtaagcggagatcgcccgacgaagcgcaaattgcttctcgtcaatcgtttcgccgcctgcataacgacttttcagcatgtttgcagcggcagataatgatgtgcacgcctggagcgcaccgtcaggtgtcagaccgagcatagaaaaatttcgagagtttatttgcatgaggccaacatccagcgaatgccgtgcatcgagacggtgcctgacgacttgggttgcttggctgtgatcttgccagtgaagcgtttcgccggtcgtgttgtcatgaatcgctaaaggatcaaagcgactctccaccttagctatcgccgcaagcgtagatgtcgcaactgatggggcacacttgcgagcaacatggtcaaactcagcagatgagagtggcgtggcaaggctcgacgaacagaaggagaccatcaaggcaagagaaagcgaccccgatctcttaagcataccttatctccttagctcgcaactaacaccgcctctcccgttggaagaagtgcgttgttttatgttgaagattatcgggagggtcggttactcgaaaattttcaattgcttctttatgatttcaattgaagcgagaaacctcgcccggcgtcttggaacgcaacatggaccgagaaccgcgcatccatgactaagcaaccggatcgacctattcaggccgcagttggtcaggtcaggctcagaacgaaaatgctcggcgaggttacgctgtctgtaaacccattcgatgaacgggaagcttccttccgattgctcttggcaggaatattggcccatgcctgcttgcgctttgcaaatgctcttatcgcgttggtatcatatgccttgtccgccagcagaaacgcactctaagcgattatttgtaaaaatgtttcggtcatgcggcggtcatgggcttgacccgctgtcagcgcaagacggatcggtcaaccgtcggcatcgacaacagcgtgaatcttggtggtcaaaccgccacgggaacgtcccatacagccatcgtcttgatcccgctgtttcccgtcgccgcatgttggtggacgcggacacaggaactgtcaatcatgacgacattctatcgaaagccttggaaatcacactcagaatatgatcccagacgtctgcctcacgccatcgtacaaagcgattgtagcaggttgtacaggaaccgtatcgatcaggaacgtctgcccagggcgggcccgtccggaagcgccacaagatgacattgatcacccgcgtcaacgcgcggcacgcgacgcggcttatttgggaacaaaggactgaacaacagtccattcgaaatcggtgacatcaaagcggggacgggttatcagtggcctccaagtcaagcctcaatgaatcaaaatcagaccgatttgcaaacctgatttatgagtgtgcggcctaaatgatgaaatcgtccttctagatcgcctccgtggtgtagcaacacctcgcagtatcgccgtgctgaccttggccagggaattgactggcaagggtgctttcacatgaccgctcttttggccgcgatagatgatttcgttgctgctttgggcacgtagaaggagagaagtcatatcggagaaattcctcctggcgcgagagcctgctctatcgcgacggcatcccactgtcgggaacagaccggatcattcacgaggcgaaagtcgtcaacacatgcgttataggcatcttcccttgaaggatgatcttgttgctgccaatctggaggtgcggcagccgcaggcagatgcgatctcagcgcaacttgcggcaaaacatctcactcacctgaaaaccactagcgagtctcgcgatcagacgaaggccttttacttaacgacacaatatccgatgtctgcatcacaggcgtcgctatcccagtcaatactaaagcggtgcaggaactaaagattactgatgacttaggcgtgccacgaggcctgagacgacgcgcgtagacagttttttgaaatcattatcaaagtgatggcctccgctgaagcctatcacctctgcgccggtctgtcggagagatgggcaagcattattacggtcttcgcgcccgtacatgcattggacgattgcagggtcaatggatctgagatcatccagaggattgccgcccttaccttccgtttcgagttggagccagcccctaaatgagacgacatagtcgacttgatgtgacaatgccaagagagagatttgcttaacccgatttttttgctcaagcgtaagcctattgaagcttgccggcatgacgtccgcgccgaaagaatatcctacaagtaaaacattctgcacaccgaaatgcttggtgtagacatcgattatgtgaccaagatccttagcagtttcgcttggggaccgctccgaccagaaataccgaagtgaactgacgccaatgacaggaatcccttccgtctgcagataggtaccatcgatagatctgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcagggggggggggggggggggttccattgttcattccacggacaaaaacagagaaaggaaacgacagaggccaaaaagctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtcccccccccccccccccctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggtcgacgatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgttattctaataaacgctcttttctcttag

SEQ ID NO: 2 - DNA, 23712 bp, synthetic construct
  source 1..23712: mol_type = other DNA
gtttacccgccaatatatcctgtcaaacactgatagtttaaactgaaggcgggaaacgacaatctgatcatgagcctagttagttaggggcagctaacgtcccctagctaactagttgctattaacctttagcctagtttagtgcggtcgcgctagtgaactagtctaatgggtgcctagcttagattagatccccgttcggcttagactaggttagtcggatagggctagtctagtggcttagcttggaactgttaagctaggctgttagtgcgggacttctaggctaacctattgacccctaacgcctagaacctaagaaacgctaaggacacctagctaactttggcgacgtcccgaggtccgattccggctaactaactagcttaagggttaccggccggccacactgatagtttaaactgaaggcgggaaacgacaatctgatcatgagcggagaattaagggagtcacgttatgacccccgccgatgacgcgggacaagccgttttacgtttggaactgacagaaccgcaacgattgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcccggcggatctcgactctagaggatcgctcaggaaggccgctgagatagagccatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgctatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccaacagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgagagcaacgcgagctgccactgctcttcactgatgtctctggaatggaaggaggaggaagtgagcatagcgttggtgcgttgctgtcaagggcgaattgtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgggtaccatttaaattgcgcccgccacggccgtggaggtcgtattccggtcagcttgcatccctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccatggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcttcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcgggcatggcggcgcgcagctcttcagtgtctggaacgacacttaagcgtacgtgccggcctggctctccgaaagggcgtattccagcacactggcggccgttactagacccaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggcctccggattcttatgtgcttctagtctccaaatgtggttgatagttattttgctctaagatcaacagtaatgaagtataaatcatcgttgtggtgtgctactcggttaattgagcattaacacacacaaacatgacgaggatggtataatctccaaaaatgtgtactttgttaggtgggaccctatagccttgattaatgtgctatgttaggcatgcctggaaacgtgtgacgcatatgttttgtgaacctgttgatattatatgtgcttttatattaccatattttattaaaatactaatatttattactagtaagatataacattctatctagcttaaaaactaaccataaatattccataataactagatttaccaaactaatatactaaatatacataataaatacaaaattaacaagacaataatcaatatttatgagcttaatatatttagacattatggttggtcgacgataatcatgctaacttttcgtaattgcttgattgaaatatgcttagaataatgcctctttgttctacatggcaaatagggaccattatggtgtaacaccctgggaaccacaaacaccccgaaatgctactaaactacacaactaaccttcatatataaaatttcgacagcatctcctttgaaaatttgcatagacgtggaagcaacagagtataaacagatatcatgataagaaaacatactagacattaataatctgctagaaatgggaagaatcacgcgtaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccaacaatgccccagttcgacatcctctgcaagaccccccccaaggtgctcgtgaggcagttcgtggagaggttcgagaggccctccggcgagaagatcgccctctgcgccgccgagctcacctacctctgctggatgatcacccacaacggcaccgccattaagagggccaccttcatgtcatacaacaccatcatctccaactccctctccttcgacatcgtgaacaagtccctccagttcaaatacaagacccagaaggccaccatcctcgaggcctccctcaagaagctcatccccgcctgggagttcaccatcatcccctactacggccagaagcaccagtccgacatcaccgacatcgtgtcatccctccagcttcagttcgagtcctccgaggaggctgacaagggcaactcccactccaagaagatgctgaaggccctcctctccgagggcgagtccatctgggagatcaccgagaagatcctcaactccttcgagtacacctccaggttcactaagaccaagaccctctaccagttcctcttcctcgccaccttcatcaactgcggcaggttctcagacatcaagaacgtggaccccaagtccttcaagctcgtgcagaacaagtacctaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtgtcatcatccagtgcctcgtgaccgagaccaagacctccgtgtccaggcacatctacttcttctccgctcgcggcaggatcgaccccctcgtgtacctcgacgagttcctcaggaactcagagcccgtgctcaagagggtgaacaggaccggcaactcctcctccaacaagcaggagtaccagctcctcaaggacaacctcgtgaggtcctacaacaaggccctcaagaagaacgccccctactccatcttcgccatcaagaacggccccaagtcccacatcggtaggcacctcatgacctccttcctctcaatgaagggcctcaccgagctcaccaacgtggtgggcaactggtccgacaagagggcctccgccgtggccaggaccacctacacccaccagatcaccgccatccccgaccactacttcgccctcgtgtcaaggtactacgcctacgaccccatctccaaggagatgatcgccctcaaggacgagactaaccccatcgaggagtggcagcacatcgagcagctcaagggctccgccgagggctccatcaggtaccccgcctggaacggcatcatctcccaggaggtgctcgactacctctcctcctacatcaacaggaggatctgagttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcggtcaccgaattcccatggagtcaaagattcaaatagaggacctaacagaactcgccgtaaagactggcgaacagttcatacagagtctcttacgactcaatgacaagaagaaaatcttcgtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactaagctgaccgaagctggccgctctagaactagtggatctcgatgtgtagtctacgagaagggttaaccgtctcttcgtgagaataaccgtggcctaaaaataagccgatgaggataaataaaatgtggtggtacagtacttcaagaggtttactcatcaagaggatgcttttccgatgagctctagtagtacatcggacctcacatacctccattgtggtgaaatattttgtgctcatttagtgatgggtaaattttgtttatgtcactctaggttttgacatttcagttttgccactcttaggttttgacaaataatttccattccgcggcaaaagcaaaacaattttattttacttttaccactcttagctttcacaatgtatcacaaatgccactctagaaattctgtttatgccacagaatgtgaaaaaaaacactcacttatttgaagccaaggtgttcatggcatggaaatgtgacataaagtaacgttcgtgtataagaaaaaattgtactcctcgtaacaagagacggaaacatcatgagacaatcgcgtttggaaggctttgcatcacctttggatgatgcgcatgaatggagtcgtctgcttgctagccttcgcctaccgcccactgagtccgggcggcaactaccatcggcgaacgacccagctgacctctaccgaccggacttgaatgcgctaccttcgtcagcgacgatggccgcgtacgctggcgacgtgcccccgcatgcatggcggcacatggcgagctcagaccgtgcgtggctggctacaaatacgtaccccgtgagtgccctagctagaaacttacacctgcaactgcgagagcgagcgtgtgagtgtagccgagtagatcccccgggctgcaggtcgactctagaggatccaccggtcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgacgaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagcggccgctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtggcgagctctcgagtcgatcgctatcaactttgtatagaaaagttgggccgaattcgagctcggtacggccagaatggcccggaccgggttaccgaattcgagctcggtaccctgggatcagcttcgctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtggcgagctgctagcgaagttcctattccgaagttcctattctctagaaagtataggaacttcagatccaccgggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggggtggaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcgaccttcttatgtgcttctagtctccaaatgtggttgatagttattttgctctaagatcaacagtaatgaagtataaatcatcgttgtggtgtgctactcggttaattgagcattaacacacacaaacatgacgaggatggtataatctccaaaaatgtgtactttgttaggtgggaccctatagccttgattaatgtgctatgttaggcatgcctggaaacgtgtgacgcatatgttttgtgaacctgttgatattatatgtgcttttatattaccatattttattaaaatactaatatttattactagtaagatataacattctatctagcttaaaaactaaccataaatattccataataactagatttaccaaactaatatactaaatatacataataaatacaaaattaacaagacaataatcaatatttatgagcttaatatatttagacattatggttggtcgacgataatcatgctaacttttcgtaattgcttgattgaaatatgcttagaataatgcctctttgttctacatggcaaatagggaccattatggtgtaacaccctgggaaccacaaacaccccgaaatgctactaaactacacaactaaccttcatatataaaatttcgacagcatctcctttgaaaatttgcatagacgtggaagcaacagagtataaacagatatcatgataagaaaacatactagacattaataatctgctagaaatgggaagaatcctaacttgacgactgcgtaactgactagagtcacacttagctgaccctagtcacttacaactgacttcgtgtcctaggcttaggctactgctagtccgcggtgtatccgtgatcgagttggcgccagacggaatctgttctccatcgctgacatcctcgagtagatcacattcaagcttgatatcgaattcctgcagcccatccctcagccgcctttcactatcttttttgcccgagtcattgtcatgtgaaccttggcatgtataatcggtgaattgcgtcgattttcctcttataggtgggccaatgaatccgtgtgatcgcgtctgattggctagagatatgtttcttccttgttggatgtattttcatacataatcatatgcatacaaatatttcattacactttatagaaatggtcagtaataaaccctatcactatgtctggtgtttcattttatttgcttttaaacgaaaattgacttcctgattcaatatttaaggatcgtcaacggtgtgcagttactaaattctggtttgtaggaactatagtaaactattcaagtcttcacttattgtgcactcacctctcgccacatcaccacagatgttattcacgtcttaaatttgaactacacatcatattgacacaatattttttttaaataagcgattaaaacctagcctctatgtcaacaatggtgtacataaccagcgaagtttagggagtaaaaaacatcgccttacacaaagttcgctttaaaaaataaagagtaaattttactttggaccacccttcaaccaatgtttcactttagaacgagtaattttattattgtcactttggaccaccctcaaatcttttttccatctacatccaatttatcatgtcaaagaaatggtctacatacagctaaggagatttatcgacgaatagtagctagcatactcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgttttttttttccgtcacggtctcgatctttggccttggtagtttgggtgggcgagaggcggcttcgtgcgcgcccagatcggtgcgcgggaggggcgggatctcgcggctggggctctcgccggcgtggatcaggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaaggatccacacgacaccatgtcccccgagcgccgccccgtcgagatccgcccggccaccgccgccgacatggccgccgtgtgcgacatcgtgaaccactacatcgagacctccaccgtgaacttccgcaccgagccgcagaccccgcaggagtggatcgacgacctggagcgcctccaggaccgctacccgtggctcgtggccgaggtggagggcgtggtggccggcatcgcctacgccggcccgtggaaggcccgcaacgcctacgactggaccgtggagtccaccgtgtacgtgtcccaccgccaccagcgcctcggcctcggctccaccctctacacccacctcctcaagagcatggaggcccagggcttcaagtccgtggtggccgtgatcggcctcccgaacgacccgtccgtgcgcctccacgaggccctcggctacaccgcccgcggcaccctgcgcgccgccggctacaagcacggcggctggcacgacgtcggcttctggcagcgcgacttcgagctgccggccccgccgcgcccggtgcgcccggtgacgcagatctgagtcgacctgcaggcatgccgctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtggcgagctaatgcggcccgaataacttcgtatagcatacattatacgaagttatacctggtggcgccgctaggggctgcaggaattcctgcagcccgggggatccactagttctagagcggccgacctcgacagatctaagcttactagtgccgtgggtcgtttaagctgccgctgtacctgtgtcgtctggtgccttctggtgtacctgggaggttgtcgtctatcaagtatctgtggttggtgtcatgagtcagtgagtcccaatactgttcgtgtcctgtgtgcattatacccaaaactgttatgggcaaatcatgaataagcttgatgttcgaacttaaaagtctctgctcaatatggtattatggttgtttttgttcgtctcctaatatttgcctgggatcaaattttattggctggtgttcatttgacctccatgttcttgctaggctccattttttactctacagccataatatgtttgattgtttggtttgttctttgttgtacacctggttctgtcgagcttagttttcgacactggcttacagcttaacatgttgctattttattgggttctgattgctattttattgggttctgattgctagtttttgctgaatccaaaaaccatgttatttatttaagcgatccaggttattattatgatggtggctaagtttttttttttccaagggtaaattttctggattctccagtgtttctgtggccgaattcactagtgattcagatctgatatcgatgggcccactaactatctatactgtaataatgttgtatagccgccggatagctagctagtttagtcattcagcggcgatgggtaataataaagtgtcatccatccatcaccatgggtggcaacgtgagcaatgacctgattgaacaaattgaaatgaaaagaagaaatatgttatatgtcaacgagatttcctcataatgccactgacgacgtgtgtccaagaaatgtatcagtgatacgtatattcacaatttttttatgacttatactcacaatttgtttttttactacttatactcacaatttgttgtgggtaccataacaatttcgatcgaatatatatcagaaagttgacgaaagtaagctcactcaaaaagttaaatgggctgcggaagctgcgtcaggcccaagttttggctattctatccggtatccacgattttgatggctgagggacatatgttcgcttaagcttggtcacccggtccgggcctagaaggccagcttcaagtttgtacaaaaaagcaggctccggccagaatctcactgactagctaaacagcggccgcatgagcatgcgcttagcagccagaatggccatctggattcagcaggcctagaaggcctcggaccgattaaactttaattcggtccggtaccatgtttaaacctaagggcgcgccattctatcactagctagctgctaattattcccgggcacccagctttcttgtacaaagtggccgttacagaatcactgactagctaatctagcggccgcgccagaagatagaagatatcctggacctgcaagatgtcagcaatgacgattgaaagattcccaggatagccggcggacgtggtggacccagtctaggtgcgatgcttagtcacgcacgatgactctgtcggaaggcatctttactttcggcaaactttaataatactttaggaaaagtattgtacaagttaggtgcagaatcaataatgcacccagctttagtcttgtctactgaattattgtgtcggttgcattattggatgcctgcgtgcaccctaagcaatccccggctctcatctctataagaggagcctttgtattcagttgcaagcatgcaagtcacacactgcaagcttacttctgagcaaaaagagttttgagtgaaataaatttgaagttcccccttacatcttgctcgagaccggtgatcttgtaaggttcccttccctcctcccctcacacccctgttcgtgttccttcggatcggatctcagtggtgatgttagacgtccgcggctgcctacgtagtggcattgccgcccgaaaggtttgtttaggtggggtagatccgaaacaggccggatctggaccatgtccgcggcggggcggcgggacttgatcgcgtagctgtcgtgtgcatttctccctaccagtggcggaatcggcgatgtggacctaagggctaaggcttatctgctgccttgaccatttcgtcgctgacaaaaacaaagtgacaatcatgccgttctctgtttgtttatctggatcgttattacgctgtgaatcctgcgatatgtggctaagtgatttttcttctttttctgggggcagtttagcctttgacccagtcctaggtgtggtcactaggactgtgtagcatgatgagtgaggttgcagcaggctgattgctagtggacgtttttttccccaatttgttaggttttcacgctccaggttgtgcaagtaattttgctagtgattgtgtgatccatcttcaacgttgaaccttgtttttccccctaaaacccccaacaggaaatcttgccccgacttctattgcaaaaattgtaacgcttagcaccctgattgactcaattcctgtcactaggcatgctcggtcaaaagcagatgatttaccacttagaaactgccctgcccctgctttccacatagcatttcgaactttttgactactattgacacccccctaacttgccgaactatttctctcttcagctactatttacctagttataattacataaatgtttgtgtgtatcttgtgcagggatccgccatgggcatcaccgtcacgaacaactcgtccaacccgatcgaggtcgccatcaaccactggggcagcgacggcgacaccagcttcttctccgtcggcaacggcaagcaggagacctgggacaggtccgacagccgcggcttcgtgctctccctgaagaagaacggcgctcagcacccgtactacgtccaggccagcagcaagatcgaggtcgacaacaacgccgtgaaggaccagggtcggctgatcgagccgctctcgtgatacgtatgtgaaaccaatctggatatccctgaaatttgaataaggttatgttgtgagtttatgaagttgtggtctagtgttgtattttgtttttcttaaggataatgcatctgtgtgctttgcttatctaaaatctaaataaagtgggtttctgcattgtgactttgcacaaacttcttttgttaaaatgaataagaaatcacatcaagaaaacagaacaagaccacaacgagaagacttgaaactctctgaaggtatataaacaaacagaacctagtaaattagagtcagcttattcaaaacttttagaccatcatcaagatcattataaacttggagtcaatgttgcggaaaccaatctgctatccccggaatttcaaataaagagactagagaattactttgaacacacttcttttattaaatcggatcacaaaaatccatcaaagacacaacagcaacaaacacaaagagaagacttaaaacactgaagcagtaaacaaacagagagaaataaagtagaatcagcttattcaaagtgtgtaaactatcatcaaaagatacttaaaatctccataaggcgcgccgtctcactagctagctgctaacgttcccgggcaactttattatacaaagttgatagatcgagcgactaattaactagctgtcccacggcctaactagcacttaatcccctagcctaacctaagagcgctaatctaggctagtggtcacttagggctttaaggctagcgtatacgaagttcctattccgaagttcctattctccagaaagtataggaacttctgtacacctgagcctaactaactaggacgtcccgaggtccgattccgggctaattaacacctagctcgccactcgactaattagggactaagcctagcgcttagccgttaaaacctagcacaccctaagcacccttagttaggttcccctcttaattaagccctagtgagcccctaagttaaggggacgctaagagccccctaacctagtattcggctagaggcgaactaggctaaacacctaagcgcacctctttaagctagatcgctagggggctaggctagagcttcgctagattagtctaagggcagctaactaactaggtttaaactacattaaaaacgtccgcaatgtgttattaagttgtctaagcgtcaatttgtttacaccacaatatatcctgcca

SEQ ID NO: 3 - DNA, 14393 bp, synthetic construct
  source 1..14393: mol_type = other DNA
actttgatgccaaacacgctctctaagtgaagctggctaaaggacaaataagggactaacatcataaaggctaacccctcaaaatcactaggatcctagattctattgtacctttgtcttctttgtgtacaacctacgtacatttgttgatgcaacttttgttacctttccgatgtcctttacatcgctttatcgtccttaccgaatgatcctctacccaaacagcgccaacgtgtacagtaccaacggttagctgaagaatagaatggtgaaggatggttggcactaggaaaacgcaaaaaaaaaaaaaacgacccaatgcatgttgcacaagggggtgtttggtttataggaagtaatttttagttcctctattttattccattttagttcataaattacaaaatgtggaaactagaatctattttagtttccatatatgtcaatttagggattaaaatagtataaaatggagggactaaaaattagtccctacaaaccaagcacccccaagtctctacatgttgacaagaatgtaataattacaagtgttttcatcggagaaagttgcattgatgatggcccaatgacagtgcacgcatgattccaacttgatggagaaatgagtagcgcgtatctcgctccatcatagtaggcggcggcagcgcatgttatgcgtgctctacgcgcataatgagggaactgtaaagtaagtagcaagaggtgtttggaaagccaggtgcatgcaaatccttgggaaaaatataatggatcctgggcgctgtctagctgaacccttttcgtatactagctagatgcatgctgcataggcattaattgggaaagccagccagccagggcgccattgtattcaagtccaattttggtccatgcatccaaccgttcggccattcgaagttcggatgtccattttcttcttttatacgtgcagtttgacgtgacatagtttcataaagtgcctgattagtgataagtgaatcaaaagagctccttgctctcctcaaagaaatttcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttactaccggctggatggcggggccttgatcgtgcaccgccggcgtccggactaactaactagtcgagctagttaccctatgaggtgacatgaagcgctcacggttactatgacggttagcttcacgactgttggtggcagtagcgtacgacttagctatagttccggacttacccttaagataacttcgtatagcatacattatacgaagttatacctggtggcgtagccggctaacgttaacaaccggtacctctagaactatagctagcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctagcgaagttcctattccgaagttcctattctctagaaagtataggaacttcagatccaccgggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggggtggaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcgaccttcttatgtgcttctagtctccaaatgtggttgatagttattttgctctaagatcaacagtaatgaagtataaatcatcgttgtggtgtgctactcggttaattgagcattaacacacacaaacatgacgaggatggtataatctccaaaaatgtgtactttgttaggtgggaccctatagccttgattaatgtgctatgttaggcatgcctggaaacgtgtgacgcatatgttttgtgaacctgttgatattatatgtgcttttatattaccatattttattaaaatactaatatttattactagtaagatataacattctatctagcttaaaaactaaccataaatattccataataactagatttaccaaactaatatactaaatatacataataaatacaaaattaacaagacaataatcaatatttatgagcttaatatatttagacattatggttggtcgacgataatcatgctaacttttcgtaattgcttgattgaaatatgcttagaataatgcctctttgttctacatggcaaatagggaccattatggtgtaacaccctgggaaccacaaacaccccgaaatgctactaaactacacaactaaccttcatatataaaatttcgacagcatctcctttgaaaatttgcatagacgtggaagcaacagagtataaacagatatcatgataagaaaacatactagacattaataatctgctagaaatgggaagaatcctaacttgacgactgcgtaactgactagagtcacacttagctgaccctagtcacttacaactgacttcgtgtcctaggcttaggctactgctagtccgcggtgtatccgtgatcgagttggcgccagacggaatctgttctccatcgctgacatcctcgagtagatcacattcaagcttgatatcgaattcctgcagcccatccctcagccgcctttcactatcttttttgcccgagtcattgtcatgtgaaccttggcatgtataatcggtgaattgcgtcgattttcctcttataggtgggccaatgaatccgtgtgatcgcgtctgattggctagagatatgtttcttccttgttggatgtattttcatacataatcatatgcatacaaatatttcattacactttatagaaatggtcagtaataaaccctatcactatgtctggtgtttcattttatttgcttttaaacgaaaattgacttcctgattcaatatttaaggatcgtcaacggtgtgcagttactaaattctggtttgtaggaactatagtaaactattcaagtcttcacttattgtgcactcacctctcgccacatcaccacagatgttattcacgtcttaaatttgaactacacatcatattgacacaatattttttttaaataagcgattaaaacctagcctctatgtcaacaatggtgtacataaccagcgaagtttagggagtaaaaaacatcgccttacacaaagttcgctttaaaaaataaagagtaaattttactttggaccacccttcaaccaatgtttcactttagaacgagtaattttattattgtcactttggaccaccctcaaatcttttttccatctacatccaatttatcatgtcaaagaaatggtctacatacagctaaggagatttatcgacgaatagtagctagcatactcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgttttttttttccgtcacggtctcgatctttggccttggtagtttgggtgggcgagaggcggcttcgtgcgcgcccagatcggtgcgcgggaggggcgggatctcgcggctggggctctcgccggcgtggatcaggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaaggatccacacgacaccatgtcccccgagcgccgccccgtcgagatccgcccggccaccgccgccgacatggccgccgtgtgcgacatcgtgaaccactacatcgagacctccaccgtgaacttccgcaccgagccgcagaccccgcaggagtggatcgacgacctggagcgcctccaggaccgctacccgtggctcgtggccgaggtggagggcgtggtggccggcatcgcctacgccggcccgtggaaggcccgcaacgcctacgactggaccgtggagtccaccgtgtacgtgtcccaccgccaccagcgcctcggcctcggctccaccctctacacccacctcctcaagagcatggaggcccagggcttcaagtccgtggtggccgtgatcggcctcccgaacgacccgtccgtgcgcctccacgaggccctcggctacaccgcccgcggcaccctgcgcgccgccggctacaagcacggcggctggcacgacgtcggcttctggcagcgcgacttcgagctgccggccccgccgcgcccggtgcgcccggtgacgcagatctgagtcgacctgcaggcatgccgctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtggcgagctaatgcggcccgaataacttcgtatagcatacattatacgaagttatacctggtggcgccgctaggggctgcaggaattcctgcagcccgggggatccactagttctagagcggccgacctcgacagatctaagcttactagtgccgtgggtcgtttaagctgccgctgtacctgtgtcgtctggtgccttctggtgtacctgggaggttgtcgtctatcaagtatctgtggttggtgtcatgagtcagtgagtcccaatactgttcgtgtcctgtgtgcattatacccaaaactgttatgggcaaatcatgaataagcttgatgttcgaacttaaaagtctctgctcaatatggtattatggttgtttttgttcgtctcctaatatttgcctgggatcaaattttattggctggtgttcatttgacctccatgttcttgctaggctccattttttactctacagccataatatgtttgattgtttggtttgttctttgttgtacacctggttctgtcgagcttagttttcgacactggcttacagcttaacatgttgctattttattgggttctgattgctattttattgggttctgattgctagtttttgctgaatccaaaaaccatgttatttatttaagcgatccaggttattattatgatggtggctaagtttttttttttccaagggtaaattttctggattctccagtgtttctgtggccgaattcactagtgattcagatctgatatcgatgggcccactaactatctatactgtaataatgttgtatagccgccggatagctagctagtttagtcattcagcggcgatgggtaataataaagtgtcatccatccatcaccatgggtggcaacgtgagcaatgacctgattgaacaaattgaaatgaaaagaagaaatatgttatatgtcaacgagatttcctcataatgccactgacgacgtgtgtccaagaaatgtatcagtgatacgtatattcacaatttttttatgacttatactcacaatttgtttttttactacttatactcacaatttgttgtgggtaccataacaatttcgatcgaatatatatcagaaagttgacgaaagtaagctcactcaaaaagttaaatgggctgcggaagctgcgtcaggcccaagttttggctattctatccggtatccacgattttgatggctgagggacatatgttcgcttaagcttggtcacccggtccgggcctagaaggccagcttcaagtttgtacaaaaaagcaggctccggccagaatctcactgactagctaaacagcggccgcatgagcatgcgcttagcagccagaatggccatctggattcagcaggcctagaaggcctcggaccgattaaactttaattcggtccggtaccatgtttaaacctaagggcgcgccattctatcactagctagctgctaattattcccgggcacccagctttcttgtacaaagtggccgttacagaatcactgactagctaatctagcggccgcgccagaagatagaagatatcctggacctgcaagatgtcagcaatgacgattgaaagattcccaggatagccggcggacgtggtggacccagtctaggtgcgatgcttagtcacgcacgatgactctgtcggaaggcatctttactttcggcaaactttaataatactttaggaaaagtattgtacaagttaggtgcagaatcaataatgcacccagctttagtcttgtctactgaattattgtgtcggttgcattattggatgcctgcgtgcaccctaagcaatccccggctctcatctctataagaggagcctttgtattcagttgcaagcatgcaagtcacacactgcaagcttacttctgagcaaaaagagttttgagtgaaataaatttgaagttcccccttacatcttgctcgagaccggtgatcttgtaaggttcccttccctcctcccctcacacccctgttcgtgttccttcggatcggatctcagtggtgatgttagacgtccgcggctgcctacgtagtggcattgccgcccgaaaggtttgtttaggtggggtagatccgaaacaggccggatctggaccatgtccgcggcggggcggcgggacttgatcgcgtagctgtcgtgtgcatttctccctaccagtggcggaatcggcgatgtggacctaagggctaaggcttatctgctgccttgaccatttcgtcgctgacaaaaacaaagtgacaatcatgccgttctctgtttgtttatctggatcgttattacgctgtgaatcctgcgatatgtggctaagtgatttttcttctttttctgggggcagtttagcctttgacccagtcctaggtgtggtcactaggactgtgtagcatgatgagtgaggttgcagcaggctgattgctagtggacgtttttttccccaatttgttaggttttcacgctccaggttgtgcaagtaattttgctagtgattgtgtgatccatcttcaacgttgaaccttgtttttccccctaaaacccccaacaggaaatcttgccccgacttctattgcaaaaattgtaacgcttagcaccctgattgactcaattcctgtcactaggcatgctcggtcaaaagcagatgatttaccacttagaaactgccctgcccctgctttccacatagcatttcgaactttttgactactattgacacccccctaacttgccgaactatttctctcttcagctactatttacctagttataattacataaatgtttgtgtgtatcttgtgcagggatccgccatgggcatcaccgtcacgaacaactcgtccaacccgatcgaggtcgccatcaaccactggggcagcgacggcgacaccagcttcttctccgtcggcaacggcaagcaggagacctgggacaggtccgacagccgcggcttcgtgctctccctgaagaagaacggcgctcagcacccgtactacgtccaggccagcagcaagatcgaggtcgacaacaacgccgtgaaggaccagggtcggctgatcgagccgctctcgtgatacgtatgtgaaaccaatctggatatccctgaaatttgaataaggttatgttgtgagtttatgaagttgtggtctagtgttgtattttgtttttcttaaggataatgcatctgtgtgctttgcttatctaaaatctaaataaagtgggtttctgcattgtgactttgcacaaacttcttttgttaaaatgaataagaaatcacatcaagaaaacagaacaagaccacaacgagaagacttgaaactctctgaaggtatataaacaaacagaacctagtaaattagagtcagcttattcaaaacttttagaccatcatcaagatcattataaacttggagtcaatgttgcggaaaccaatctgctatccccggaatttcaaataaagagactagagaattactttgaacacacttcttttattaaatcggatcacaaaaatccatcaaagacacaacagcaacaaacacaaagagaagacttaaaacactgaagcagtaaacaaacagagagaaataaagtagaatcagcttattcaaagtgtgtaaactatcatcaaaagatacttaaaatctccataaggcgcgccgtctcactagctagctgctaacgttcccgggcaactttattatacaaagttgatagatcgagcgactaattaactagctgtcccacggcctaactagcacttaatcccctagcctaacctaagagcgctaatctaggctagtggtcacttagggctttaaggctagcgtatacgaagttcctattccgaagttcctattctccagaaagtataggaacttctgtacacctgagctgattccgatgacttcgtaggttcctagctcaagccgctcgtgtccaagcgtcacttacgattagctaatgattacggcatctaggaccgactagctaactaactagtacgtagaattaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacatacacgtacacgcatttggtacagctaaccgtgagcagcgcgcagcaataggagattaggaggtacgtaggggggctccgaaccgttccaaattgtaaaggtgtttttatatttttcagagaaataaaatatctcaaatttaacagaagtacatgaaaaagcacaaagttttacattggtacagtgtgtggcgggcgggagggaacatatatgtgcagtgcagtgacactaggcagtgtaggccacgccgacagatgggtacactgatggatggatatgggtgcgcactcatgcatcctccccatacatactcgatcccatgcatatggtgctgttcattttaggggattttcccttttattttctttattgaaaatataagaaatgaaattttaaaaaaaaaggaaatgggagcctgctgggccgctgccatgccgcttggccgagcagcccagcagggcagccggctggtaacaaaaggtagggcacgtcgtgccttacgcaggggcaggaggcaagcggccagggggcctgtggcggcggcagcttgtgatgttccgatctgggcggcgtatcctgccggagctctcgcgatatggttgttgctcctcctctcggctccatatatggaggcttgttcccctggctggctctccacgggagagaagctggttctctgttcttctctcactcactgtttcaatcgcaactcgctcgatccactgttccaccagtggctgctccacggaggttgtgctgctcgtgcgcagtgaggctctccggtgctctattgcttcaccagtgcgcgtgtgtgctgcggttgattctctggtctctgtccaccgtggtctggtgcttgggctccctgctgcgcggtgttcctcttctcgggccggtgccgtggacgtctccctcggctccggcagcggctctattatctccgtcaacctcccgccgtgcaggtccggacgtgggcggcggattggttttctgggacagaacctcgtgttcgctgagctggtcttcccacgtagacaatctacgtgctgtgagttacccgttgcctcgctttatttgttgagtaattttgtgcaatctgctagtattatttacttgggtgtgattgcacaatttatgggcggatttgattgctcgatg

SEQ ID NO: 4 - DNA, 261 bp, Pseudomonas chlororaphis
  source 1..261: mol_type = other DNA
atgggcatcaccgtcacgaacaactcgtccaacccgatcgaggtcgccatcaaccactggggcagcgacggcgacaccagcttcttctccgtcggcaacggcaagcaggagacctgggacaggtccgacagccgcggcttcgtgctctccctgaagaagaacggcgctcagcacccgtactacgtccaggccagcagcaagatcgaggtcgacaacaacgccgtgaaggaccagggtcggctgatcgagccgctctcgtga

SEQ ID NO: 5 - protein, 86 aa, Pseudomonas chlororaphis
MGITVTNNSSNPIEVAINHWGSDGDTSFFSVGNGKQETWDRSDSRGFVLSLKKNGAQHPYYVQASSKIEVDNNAVKDQGRLIEPLS

SEQ ID NO: 6 - DNA, 27 bp, synthetic construct
  source 1..27: mol_type = other DNA
cgcaatgtgttattaagttgtctaagc

SEQ ID NO: 7 - DNA, 22 bp, synthetic construct
  source 1..22: mol_type = other DNA
cacggttagctgtaccaaatgc

SEQ ID NO: 8 - DNA, 21 bp, synthetic construct
  source 1..21: mol_type = other DNA
tttgtttacaccacatacacg

SEQ ID NO: 9 - DNA, 19 bp, synthetic construct
  source 1..19: mol_type = other DNA
acaacaacgccgtgaagga

SEQ ID NO: 10 - DNA, 25 bp, synthetic construct
  source 1..25: mol_type = other DNA
ccagattggtttcacatacgtatca

SEQ ID NO: 11 - DNA, 14 bp, synthetic construct
  source 1..14: mol_type = other DNA
agggtcggctgatc

SEQ ID NO: 12 - DNA, 19 bp, synthetic construct
  source 1..19: mol_type = other DNA
tgactgtcaaaggccacgg

SEQ ID NO: 13 - DNA, 24 bp, synthetic construct
  source 1..24: mol_type = other DNA
agatggacaagtctaggttccacc

SEQ ID NO: 14 - DNA, 28 bp, synthetic construct
  source 1..28: mol_type = other DNA
ccgtttagcgcgtgtttacaacaagctg

SEQ ID NO: 15 - DNA, 24 bp, synthetic construct
  source 1..24: mol_type = other DNA
catcgtgaaccactacatcgagac

SEQ ID NO: 16 - DNA, 18 bp, synthetic construct
  source 1..18: mol_type = other DNA
gtcgatccactcctgcgg

SEQ ID NO: 17 - DNA, 22 bp, synthetic construct
  source 1..22: mol_type = other DNA
accgtgaacttccgcaccgagc

SEQ ID NO: 18 - DNA, 23 bp, synthetic construct
  source 1..23: mol_type = other DNA
ttggactagaaatctcgtgctga

SEQ ID NO: 19 - DNA, 22 bp, synthetic construct
  source 1..22: mol_type = other DNA
gctacatagggagccttgtcct

SEQ ID NO: 20 - DNA, 16 bp, synthetic construct
  source 1..16: mol_type = other DNA
gcgtttgtgtggattg

SEQ ID NO: 21 - DNA, 80 bp, synthetic construct
  source 1..80: mol_type = other DNA
cgcaatgtgttattaagttgtctaagcgtcaatttgtttacaccacatacacgtacacgcatttggtacagctaaccgtg

SEQ ID NO: 22 - DNA, 72 bp, synthetic construct
  source 1..72: mol_type = other DNA
acaacaacgccgtgaaggaccagggtcggctgatcgagccgctctcgtgatacgtatgtgaaaccaatctgg

SEQ ID NO: 23 - DNA, 113 bp, synthetic construct
  source 1..113: mol_type = other DNA
tgactgtcaaaggccacggccgtttagcgcgtgtttacaacaagctgtaagagcttactgaaaaaattaacatctcttgctaagctgggggtggaacctagacttgtccatct

SEQ ID NO: 24 - DNA, 76 bp, synthetic construct
  source 1..76: mol_type = other DNA
catcgtgaaccactacatcgagacctccaccgtgaacttccgcaccgagccgcagaccccgcaggagtggatcgac

SEQ ID NO: 25 - DNA, 79 bp, synthetic construct
  source 1..79: mol_type = other DNA
ttggactagaaatctcgtgctgattaattgttttacgcgtgcgtttgtgtggattgtaggacaaggctccctatgtagc

SEQ ID NO: 26 - DNA, 10 bp, synthetic construct
  source 1..10: mol_type = other DNA
aaatttcaaa

SEQ ID NO: 27 - DNA, 20 bp, synthetic construct
  source 1..20: mol_type = other DNA
caaagaaatttcaaacactg

SEQ ID NO: 28 - DNA, 50 bp, synthetic construct
  source 1..50: mol_type = other DNA
gctccttgctctcctcaaagaaatttcaaacactgatagtttaaactgaa

SEQ ID NO: 29 - DNA, 10 bp, synthetic construct
  source 1..10: mol_type = other DNA
ccacatacac

SEQ ID NO: 30 - DNA, 20 bp, synthetic construct
  source 1..20: mol_type = other DNA
ttacaccacatacacgtaca

SEQ ID NO: 31 - DNA, 50 bp, synthetic construct
  source 1..50: mol_type = other DNA
taagcgtcaatttgtttacaccacatacacgtacacgcatttggtacagc